(19) United States
(12) Patent Application Publication
(10) Pub. No.: US 2016/0017024 A1
(43) Pub. Date: Jan. 21, 2016

(54) FAM150A, FAM150B, AND FAM150 ANTAGONISTS AND USES THEREOF

(71) Applicant: FIVE PRIME THERAPEUTICS, INC., South San Francisco, CA (US)

(72) Inventors: Lewis T. Williams, Mill Valley, CA (US); Hongbing Zhang, Albany, CA (US); Kevin Hester, Kensington, CA (US); Thomas Harding, South San Francisco, CA (US)

(21) Appl. No.: 14/773,329

(22) PCT Filed: Mar. 7, 2014
(86) PCT No.: PCT/US14/21857

§ 371 Date: Sep. 5, 2015

Related U.S. Application Data
(60) Provisional application No. 61/777,034, filed on Mar. 12, 2013.

(51) Int. Cl. C07K 16/18; C07K 16/40; G01N 33/573; A61K 45/06; A61K 39/395; A61K 38/17; C07K 16/00; A61K 38/45

(52) U.S. Cl. CPC C07K 16/18; C07K 16/00; C07K 16/40; A61K 38/45; A61K 45/06; A61K 39/3955; A61K 38/1703; G01N 33/573; G01N 2500/02; G01N 2440/14; G01N 2333/912; G01N 2333/4706; G01N 2500/10; C12Y 207/10001

(57) ABSTRACT

Methods of identifying and using FAM150A agents, FAM150B agents, and FAM150 antagonists are provided. Methods of identifying and using LTK agonists (including LTK agonist antibodies, FAM150A agents, and FAM150B agents) and FAM150 antagonists are provided. Such methods include, but are not limited to, methods of treating cancer, methods of treating immune disorders such as autoimmune diseases, and methods of treating neurodegenerative diseases.

### TECHNICAL FIELD

Methods of identifying and using LTK agonists (including LTK agonist antibodies, FAM150A agents, and FAM150B agents) and FAM150 antagonists are provided. Such methods include, but are not limited to, methods of treating cancer, methods of treating immune disorders such as autoimmune diseases, and methods of treating neurodegenerative diseases. FAM150A agents include FAM150A and FAM150A fusion molecules. FAM150B agents include FAM150B and FAM150B fusion molecules. FAM150 antagonists include, but are not limited to, antibodies that bind FAM150A and inhibit FAM150A-mediated signaling (such as, for example, by blocking binding of FAM150A to leukocyte tyrosine kinase (LTK)); antibodies that bind FAM150B and inhibit FAM150B-mediated signaling (such as, for example, by blocking binding of FAM150B to LTK); antibodies that bind both FAM150A and FAM150B and inhibit both FAM150A- and FAM150B-mediated signaling; antibodies that bind LTK and inhibit FAM150A- and/or FAM150B-mediated signaling (such as, for example, by blocking binding of FAM150A and/or FAM150B to the receptor); and soluble forms of LTK.

### BACKGROUND

Leukocyte tyrosine kinase (LTK) is a receptor tyrosine kinase that has been shown to be expressed in various hematopoietic cells, in brain and placenta, and in various cancer cells. Full-length LTK is a 100 kDa glycosylated protein, but several splice variant forms have also been described. Studies have shown that LTK plays a role in growth and development. In mice, expression of aberrantly activated LTK leads to cardiac hypertrophy and cardiomyocyte degeneration (Honda et al., 1999, Oncogene, 18: 3821-3830). In zebrafish, LTK is proposed to be involved in fate specification of neural crest cells (Lopes et al., 2008, PLoS Genet., 4: e1000026). In pro-B cells expressing an EGFR/LTK chimera, LTK has been shown to associate with both IRS-1 and Shc and to activate the RAS pathway and mitogenic signaling (Ueno et al., 1997, Oncogene, 14: 3067-3072). LTK associates with PI3K, an interaction that is required for LTK to promote survival in hematopoietic cells (Ueno et al., 1997, Oncogene, 14: 3067-3072).

Surprisingly, two decades after its cloning, a ligand for LTK has not yet been identified. Therefore, it would be highly advantageous to identify ligand(s) for LTK to produce therapeutic ligands that stimulate LTK activity and/or therapeutic antagonists that inhibit LTK activity.

### SUMMARY

In some embodiments, methods of inhibiting ligand-induced phosphorylation of LTK in a subject are provided. In some embodiments, the a method comprises administering to the subject at least one molecule selected from a FAM150A antagonist, a FAM150B antagonist, and a FAM150A/B antagonist. In some embodiments, methods of inhibiting ligand-induced phosphorylation of LTK in a cell are provided. In some embodiments, a method comprises contacting the cell with at least one molecule selected from a FAM150A antagonist, a FAM150B antagonist, and a FAM150A/B antagonist. In some embodiments, the cell is in vitro.

In some embodiments, methods of inhibiting binding of FAM150A and/or FAM150B to LTK in a subject are provided. In some embodiments, a method comprise administering to the subject at least one molecule selected from a FAM150A antagonist, a FAM150B antagonist, and a FAM150A/B antagonist. In some embodiments, methods of inhibiting binding of FAM150A and/or FAM150B to LTK in a cell are provided. In some embodiments, a method comprises contacting the cell with at least one molecule selected from a FAM150A antagonist, a FAM150B antagonist, and a FAM150A/B antagonist. In some embodiments, the cell is in vitro.

In some embodiments, methods of treating cancer are provided. In some embodiments, a method comprises administering to a subject with cancer an effective amount of at least one molecule selected from a FAM150A antagonist, a FAM150B antagonist, and a FAM150A/B antagonist. In some embodiments, the cancer is selected from lung cancer, leukemia, breast cancer, ovarian cancer, kidney cancer, colon cancer, and bladder cancer. In some embodiments, the cancer is selected from breast invasive carcinoma, ovarian serous cystadenocarcinoma, kidney renal clear cell carcinoma, colon adenocarcinoma, bladder urothelial carcinoma, lung squamous cell carcinoma, non-small lung cancer, acute myeloid leukemia, and chronic lymphocytic leukemia. In some embodiments, the cancer is selected from non-small lung cancer, acute myeloid leukemia, and chronic lymphocytic leukemia. In some embodiments, the method further comprises administering to the subject an effective amount of a therapeutic agent selected from chemotherapeutic agents, anti-angiogenesis agents, growth inhibitory agents, and anti-neoplastic compositions.

In some embodiments, methods of treating autoimmune conditions are provided. In some embodiments, a method comprises administering to a subject with the autoimmune condition an effective amount of at least one molecule selected from a FAM150A antagonist, a FAM150B antagonist, and a FAM150A/B antagonist. In some embodiments, the autoimmune condition is selected from rheumatoid arthritis, systemic lupus erythematosus, ankylosing spondylitis, and multiple sclerosis. In some embodiments, the method further comprises administering to the subject an effective amount of a pharmaceutical agent selected from DMARDs, TNF inhibitors and immunosuppressive agents.

In any of the embodiments described herein, a method may comprise administering a FAM150A antagonist selected from a FAM150A antibody, a leukocyte tyrosine kinase (LTK) antibody, an LTK extracellular domain (ECD), an LTK ECD fusion molecule, and an ALK antibody. In any of the embodiments described herein, a method may comprise administering a FAM150B antagonist selected from a FAM150B antibody, a leukocyte tyrosine kinase (LTK) antibody, an LTK extracellular domain (ECD), an LTK ECD fusion molecule, and an ALK antibody. In any of the embodiments described herein, a method may comprise administering a FAM150A/B antagonist selected from a FAM150A/B antibody, a leukocyte tyrosine kinase (LTK) antibody, an LTK extracellular domain (ECD), an LTK ECD fusion molecule, and an ALK antibody. In any of the embodiments described herein, a method may comprise administering at least one molecule selected from a FAM150A antibody, a FAM150B antibody, and a FAM150A/B antibody. In any of the embodiments described herein, an antibody may be selected from a chimeric antibody, a humanized antibody, and a human antibody. In any of the embodiments described herein, an antibody may be an antibody fragment. In some embodiments, the antibody fragment is selected from an Fv, a single-chain Fv (scFv), a Fab, a Fab′, and a (Fab′)₂.

In any of the embodiments described herein, a method may comprise administering an LTK ECD. In some embodiments, the LTK ECD comprises a sequence selected from SEQ ID NOs: 13, 14, 30, and 31. In any of the embodiments described herein, a method may comprise administering an LTK ECD fusion molecule. In some embodiments, the LTK ECD fusion molecule comprises an LTK ECD and at least one fusion partner. In some embodiments, at least one fusion partner is selected from an Fc, albumin, and polyethylene glycol. In some embodiments, at least one fusion partner is an Fc. In some embodiments, the Fc comprises a sequence selected from SEQ ID NOs: 17 to 19. In some embodiments, at least one fusion partner is polyethylene glycol. In some embodiments, the LTK ECD portion of the LTK ECD fusion molecule comprises a sequence selected from SEQ ID NOs: 13, 14, 30, and 31.

In some embodiments, methods of increasing ligand-induced phosphorylation of LTK in a subject are provided. In some embodiments, a method comprises administering at least one LTK agonist to the subject. In some embodiments, methods of increasing neuronal differentiation in a subject are provided. In some embodiments, a method comprises administering at least one at least one LTK agonist to the subject. In some embodiments, methods of increasing ligand-induced phosphorylation of LTK in a cell are provided. In some embodiments, a method comprises contacting the cell with at least one LTK agonist. In some embodiments, the cell is in vitro.

In some embodiments, methods of treating neurodegenerative disorders are provided. In some embodiments, a method comprises administering at least one at least one LTK agonist to a subject with a neurodegenerative disorder. In some embodiments, the neurodegenerative disorder is selected from Huntington's disease, Parkinson's disease, and Alzheimer's disease. In some embodiments, a method of treating a neurodegenerative disorder further comprises administering a therapeutic agent selected from cholinesterase inhibitors, such as donepezil (Aricept®), galantamine (Razadyne®), and rivastigmine) (Exelon®); memantine (Namenda®); tetrabenazine (Xenazine®), antipsychotic agents, such as haloperidol (Haldol®) and clozapine, clonazepam (Klonapin®), and diazepam; antidepressants, such as escitalopram (Lexapro®), fluoxetine (Prozac®, Sarafem®) and sertraline (Zoloft®); anti-psychotic agents, such as lithium (Lithobid®); and anticonvulsants, such as valproic acid (Depakene®), divalproex (Depakote®), and lamotrigine (Lamictal®); carbidopa-levodopa (Parcopa®); dopamine agonists, such as pramipexole (Mirapex®), ropinirole (Requip®), and apomorphine (Apokyn®); monoamine oxidase B inhibitors, such as selegiline (Eldepryl®, Zelapar®) and rasagiline (Azilect®); catechol O-methyltransferase (COMT) inhibitors, such as entacapone (Comtan®) and tolcapone (Tasmar®); anticholinergics, such as benztropine (Cogentin®) and trihexyphenidyl; and amantadine.

In some embodiments in which the neurodegenerative disorder is Alzheimer's disease, the method further comprises administering a therapeutic agent selected from cholinesterase inhibitors, such as donepezil (Aricept®), galantamine (Razadyne®), and rivastigmine (Exelon®); and memantine (Namenda®). In some embodiments in which the neurodegenerative disorder is Huntington's disease, the method further comprises administering a therapeutic agent selected from agents to treat movement disorders, such as tetrabenazine (Xenazine®), antipsychotic agents, such as haloperidol (Haldol®) and clozapine, clonazepam (Klonapin®), and diazepam; antidepressants, such as escitalopram (Lexapro®), fluoxetine (Prozac®, Sarafem®) and sertraline (Zoloft®); anti-psychotic agents, such as lithium (Lithobid®); and anticonvulsants, such as valproic acid (Depakene®), divalproex (Depakote®), and lamotrigine (Lamictal®). In some embodiments in which the neurodegenerative disorder is Parkinson's disease, the method further comprises administering a therapeutic agent selected from carbidopa-levodopa (Parcopa®); dopamine agonists, such as pramipexole (Mirapex®), ropinirole (Requip®), and apomorphine (Apokyn®); monoamine oxidase B inhibitors, such as selegiline (Eldepryl®, Zelapar®) and rasagiline (Azilect®); catechol O-methyltransferase (COMT) inhibitors, such as entacapone (Comtan®) and tolcapone (Tasmar®); anticholinergics, such as benztropine (Cogentin®) and trihexyphenidyl; and amantadine.

In any of the embodiments described herein, at least one LTK agonist may be selected from an LTK agonist antibody, a FAM150A agent, and a FAM150B agent. In any of the embodiments described herein, at least one LTK agonist may be selected from a FAM150A agent and a FAM150B agent. In any of the embodiments described herein, at least one LTK agonist may be a FAM150A agent. In some embodiments, the FAM150A agent comprises a sequence selected from SEQ ID NOs: 1 and 2. In any of the embodiments described herein, at least one LTK agonist may be a FAM150B agent. In some embodiments, the FAM150B agent comprises a sequence selected from SEQ ID NOs: 3 and 4. In any of the embodiments described herein, at least one LTK agonist may be a FAM150A fusion molecule. In some embodiments, the FAM150A fusion molecule comprises FAM150A and at least one fusion partner. In any of the embodiments described herein, at least one LTK agonist may be a FAM150B fusion molecule. In some embodiments, the FAM150B fusion molecule comprises FAM150B and at least one fusion partner. In some embodiments of FAM150A fusion molecules and FAM150B fusion molecules, at least one fusion partner is selected from an Fc, albumin, and polyethylene glycol. In some embodiments, at least one fusion partner is an Fc. In some embodiments, the Fc comprises a sequence selected from SEQ ID NOs: 17 to 19. In some embodiments, at least one fusion partner is polyethylene glycol.

In some embodiments, uses of molecules selected from FAM150A antagonists, FAM150B antagonists, and FAM150A/B antagonists for treating cancer in subjects are provided. In some embodiments, the cancer is selected from lung cancer, leukemia, breast cancer, ovarian cancer, kidney cancer, colon cancer, and bladder cancer. In some embodiments, the cancer is selected from breast invasive carcinoma, ovarian serous cystadenocarcinoma, kidney renal clear cell carcinoma, colon adenocarcinoma, bladder urothelial carcinoma, lung squamous cell carcinoma, non-small lung cancer, acute myeloid leukemia, and chronic lymphocytic leukemia. In some embodiments, the cancer is selected from non-small lung cancer, acute myeloid leukemia, and chronic lymphocytic leukemia.

In some embodiments, uses of molecules selected from FAM150A antagonists, FAM150B antagonists, and FAM150A/B antagonists for treating autoimmune conditions in subjects are provided. In some embodiments, the autoimmune condition is selected from rheumatoid arthritis, systemic lupus erythematosus, ankylosing spondylitis, and multiple sclerosis. In any of the uses described herein, the FAM150A antagonist may be selected from a FAM150A antibody, a leukocyte tyrosine kinase (LTK) antibody, an LTK extracellular domain (ECD), and an LTK ECD fusion molecule; the FAM150B antagonist is selected from a FAM150B antibody, a leukocyte tyrosine kinase (LTK) antibody, an LTK extracellular domain (ECD), and an LTK ECD fusion molecule; and the FAM150A/B antagonist is selected from a FAM150A/B antibody, a leukocyte tyrosine kinase (LTK) antibody, an LTK extracellular domain (ECD), an LTK ECD fusion molecule, and an ALK antibody. In any of the uses described herein, the FAM150A antagonist may be a FAM150A antibody, the FAM150B antagonist may be a FAM150B antibody, and the FAM150A/B antagonist may be a FAM150A/B antibody. In some embodiments, the antibody is selected from a chimeric antibody, a humanized antibody, and a human antibody. In some embodiments, the antibody is an antibody fragment. In some embodiments, the antibody fragment is selected from an Fv, a single-chain Fv (scFv), a Fab, a Fab′, and a (Fab′)₂.

In some embodiments, uses of at least one LTK agonist for treating neurodegenerative disorders are provided. In some embodiments, the neurodegenerative disorder is selected from Huntington's disease, Parkinson's disease, and Alzheimer's disease. In any of the uses described herein, at least one LTK agonist may be selected from an LTK agonist antibody, a FAM150A agent, and a FAM150B agent. In any of the uses described herein, at least one LTK agonist may be selected from a FAM150A agent and a FAM150B agent. In some embodiments, the FAM150A agent comprises a sequence selected from SEQ ID NOs: 1 and 2; and the FAM150B agent comprises a sequence selected from SEQ NOs: 3 and 4. In some embodiments, the FAM150A agent is a FAM150A fusion molecule comprising FAM150A and at least one fusion partner; and wherein the FAM150B agent is a FAM150B fusion molecule comprising FAM150B and at least one fusion partner. In some embodiments, at least one fusion partner is selected from an Fc, albumin, and polyethylene glycol. In some embodiments, at least one fusion partner is an Fc.

In some embodiments, methods of identifying FAM150 antagonists are provided. In some embodiments, a method comprises contacting a candidate molecule with an LTK molecule and a FAM150 molecule, wherein the LTK molecule comprises LTK, an LTK ECD, or an LTK ECD fusion molecule, and the FAM150 molecule is selected from a FAM150A agent and a FAM150B agent. In some embodiments, a method comprises forming a composition comprising a candidate molecule, an LTK molecule, and a FAM150 molecule, wherein the LTK molecule comprises LTK, an LTK ECD, or an LTK ECD fusion molecule, and the FAM150 molecule is selected from a FAM150A agent and a FAM150B agent. In some embodiments, a method further comprises detecting binding of the LTK molecule to the FAM150 molecule. In some embodiments, a reduction in the binding of the LTK molecule to the FAM150 molecule in the presence of the candidate molecule as compared to the binding of the LTK molecule to the FAM150 molecule in the absence of the candidate molecule indicates that the candidate molecule is a FAM150 antagonist. In some embodiments, binding of the LTK molecule to the FAM150 molecule is reduced by at least 30%, at least 40%, at least 50%, at least 60%, at least 70%, or at least 80% in the presence of the candidate molecule. In some embodiments, binding of the LTK molecule to the FAM150 molecule is detected by a method selected from surface plasmon resonance, ELISA, and flow cytometry.

In some embodiments, methods of identifying FAM150 antagonists are provided, wherein a method comprises contacting a candidate molecule with a cell expressing LTK and a FAM150 molecule, wherein the FAM150 molecule is selected from a FAM150A agent and a FAM150B agent. In some embodiments, methods of identifying FAM150 antagonists are provided, wherein a method comprises forming a composition comprising a candidate molecule, a cell expressing LTK, and a FAM150 molecule, wherein the FAM150 molecule is selected from a FAM150A agent and a FAM150B agent. In some embodiments, a method further comprises detecting phosphorylation of LTK. In some embodiments, a reduction in phosphorylation of LTK in the presence of the candidate molecule as compared to the level of phosphorylation of LTK in the presence of the FAM150 molecule and the absence of the candidate molecule indicates that the candidate molecule is a FAM150 antagonist. In some embodiments, phosphorylation of LTK is reduced by at least 30%, at least 40%, at least 50%, at least 60%, at least 70%, or at least 80% in the presence of the candidate molecule. In some embodiments, phosphorylation of LTK is detected by a method selected from an immunoassay and a reporter assay.

In any of the methods of identifying FAM150 antagonists described herein, the FAM150 antagonist may be an antibody that binds to LTK. In any of the methods of identifying FAM150 antagonists described herein, the FAM150 antagonist may be an antibody that binds FAM150A and/or FAM150B. In any of the methods of identifying FAM150 antagonists described herein, the FAM150 antagonist may be a small molecule.

In some embodiments, methods of determining whether an LTK antibody is a FAM150 antagonist are provided. In some embodiments, a method comprises contacting the LTK antibody with an LTK molecule and a FAM150 molecule, wherein the LTK molecule comprises LTK, an LTK ECD, or an LTK ECD fusion molecule, and the FAM150 molecule is selected from a FAM150A agent and a FAM150B agent. In some embodiments, a method comprises forming a composition comprising the LTK antibody, an LTK molecule, and a FAM150 molecule, wherein the LTK molecule comprises LTK, an LTK ECD, or an LTK ECD fusion molecule, and the FAM150 molecule is selected from a FAM150A agent and a FAM150B agent. In some embodiments, a method further comprises detecting the binding of the LTK molecule to the FAM150 molecule. In some embodiments, a reduction in the binding of the LTK molecule to the FAM150 molecule in the presence of the LTK antibody as compared to the binding of the LTK molecule to the FAM150 molecule in the absence of the LTK antibody indicates that the LTK antibody is a FAM150 antagonist. In some embodiments, binding of the LTK molecule to the FAM150 molecule is reduced by at least 30%, at least 40%, at least 50%, at least 60%, at least 70%, or at least 80% in the presence of the LTK antibody. In some embodiments, binding of the LTK molecule to the FAM150 molecule is detected by a method selected from surface plasmon resonance, ELISA, and flow cytometry.

In some embodiments, methods of determining whether an LTK antibody is a FAM150 antagonist are provided, wherein a method comprises contacting the LTK antibody with a cell expressing LTK and a FAM150 molecule, wherein the FAM150 molecule is selected from a FAM150A agent and a FAM150B agent. In some embodiments, methods of determining whether an LTK antibody is a FAM150 antagonist are provided, wherein a method comprises forming a composition comprising the LTK antibody, a cell expressing LTK, and a FAM150 molecule, wherein the FAM150 molecule is selected from a FAM150A agent and a FAM150B agent. In some embodiments, a method further comprises detecting phosphorylation of LTK. In some embodiments, a reduction in phosphorylation of LTK in the presence of the LTK antibody as compared to the level of phosphorylation of LTK in the presence of the FAM150 molecule and the absence of the LTK antibody indicates that the LTK antibody is a FAM150 antagonist. In some embodiments, phosphorylation of LTK is reduced by at least 30%, at least 40%, at least 50%, at least 60%, at least 70%, or at least 80% in the presence of the LTK antibody. In some embodiments, phosphorylation of LTK is detected by a method selected from an immunoassay and a reporter assay.

In some embodiments, methods of determining whether an ALK antibody is a FAM150 antagonist are provided. In some embodiments, a method comprises contacting the ALK antibody with an ALK molecule and a FAM150 molecule, wherein the ALK molecule is selected from ALK, an ALK ECD, and an ALK ECD fusion molecule, and the FAM150 molecule is selected from a FAM150A agent and a FAM150B agent. In some embodiments, a method comprises forming a composition comprising the ALK antibody, an ALK molecule, and a FAM150 molecule, wherein the ALK molecule is selected from ALK, an ALK ECD, and an ALK ECD fusion molecule, and the FAM150 molecule is selected from a FAM150A agent and a FAM150B agent. In some embodiments, a method further comprises detecting the binding of the ALK molecule to the FAM150 molecule. In some embodiments, a reduction in the binding of the ALK molecule to the FAM150 molecule in the presence of the ALK antibody as compared to the binding of the ALK molecule to the FAM150 molecule in the absence of the ALK antibody indicates that the ALK antibody is a FAM150 antagonist. In some embodiments, binding of the ALK molecule to the FAM150 molecule is reduced by at least 30%, at least 40%, at least 50%, at least 60%, at least 70%, or at least 80% in the presence of the ALK antibody. In some embodiments, binding of the ALK molecule to the FAM150 molecule is detected by a method selected from surface plasmon resonance, ELISA, and flow cytometry.

In some embodiments, methods of determining whether an ALK antibody is a FAM150 antagonist are provided, wherein a method comprises contacting the ALK antibody with a cell expressing ALK and a FAM150 molecule, wherein the FAM150 molecule is selected from a FAM150A agent and a FAM150B agent. In some embodiments, methods of determining whether an ALK antibody is a FAM150 antagonist are provided, wherein a method comprises forming a composition comprising the ALK antibody, a cell expressing ALK, and a FAM150 molecule, wherein the FAM150 molecule is selected from a FAM150A agent and a FAM150B agent. In some embodiments, a method further comprises detecting phosphorylation of ALK. In some embodiments, a reduction in phosphorylation of ALK in the presence of the ALK antibody as compared to the level of phosphorylation of ALK in the presence of the FAM150 molecule and the absence of the ALK antibody indicates that the ALK antibody is a FAM150 antagonist. In some embodiments, phosphorylation of ALK is reduced by at least 30%, at least 40%, at least 50%, at least 60%, at least 70%, or at least 80% in the presence of the ALK antibody. In some embodiments, phosphorylation of ALK is detected by a method selected from an immunoassay and a reporter assay.

Any embodiment described herein or any combination thereof applies to any and all methods of the invention described herein.

### BRIEF DESCRIPTION OF THE FIGURES

FIGS. 1A and B show induction of LTK phosphorylation by (A) FAM150A and (B) FAM150B, as described in Example 4.

FIG. 2 shows dose-dependent induction of LTK phosphorylation by FAM150A, as described in Example 4.

FIG. 3 shows induction of LTK phosphorylation in the presence of FAM150A, and inhibition of FAM150A-induced LTK phosphorylation by kinase inhibitor crizotinib, as described in Example 4.

FIGS. 4A and B show mRNA expression of (A) FAM150A and (B) FAM150B in various cell lines, as described in Example 6.

FIG. 5A to C show mRNA expression of (A) FAM150A, (B) FAM150B, and (C) LTK in a panel of human immune cells, as described in Example 6.

FIG. 6A to D show (A and B) PC12 cells transfected with CMV-EGFP 72 hours post-transfection under (A) bright field and (B) fluorescence microscopy; and (C and D) PC12 cells transfected with CMV-EGFP contacted with nerve growth factor (7S NGF) after 7 days under (C) bright field and (D) fluorescence microscopy, as described in Example 7.

FIG. 7A to D show neurite outgrowth in PC12 cells (A) transfected with LTK, (B) contacted with 1 μg/ml FAM150A without LTK transfection, (C) contacted with 100 ng/ml 7S NGF, and (D) transfected with LTK and contacted with 1 μg/ml FAM150A, as described in Example 7. Arrows point to exemplary neurite outgrowth.

FIG. 8 shows an alignment of FAM150A and FAM150B.

### DETAILED DESCRIPTION

The present inventors have now identified two ligands of human LTK, FAM150A and FAM150B. Contacting LTK-expressing cells with FAM150A or FAM150B increases phosphorylation of LTK, which leads to downstream signaling. Thus, targeting the interaction between FAM150A and/or FAM150B and LTK should reduce phosphorylation of LTK, inhibiting downstream signaling. Targeting molecules include antibodies that bind FAM150A, antibodies that bind FAM150B, antibodies that bind both FAM150A and FAM150B, antibodies that bind LTK that block the binding of FAM150A and/or FAM150B, and soluble LTK extracellular domains. LTK is expressed in various cancers, and in T cells and plasmacytoid dendritic cells. Reducing or inhibiting signaling through LTK by administering a FAM150 antagonist may therefore be an effective treatment for cancer and various autoimmune diseases, such as lupus erythematosus, multiple sclerosis, rheumatoid arthritis, and ankylosing spondylitis.

Contacting PC12 cells transfected with LTK with FAM150A induces neurite outgrowth and differentiation. Thus, increasing signaling through LTK by administering an LTK agonist (such as an LTK agonist antibody, a FAM150A agent, and/or a FAM150B agent) may be effective for treating neurodegenerative diseases, such as Parkinson's disease, Huntington's disease, and Alzheimer's disease.

All references cited herein, including patent applications and publications, are incorporated by reference herein in their entirety.

The section headings used herein are for organizational purposes only and are not to be construed as limiting the subject matter described.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Exemplary techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (3rd ed., Cold Spring Harbor Laboratory Press, Cold Spring Harbor, N.Y. (2001)), among other places. In addition, exemplary techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Unless otherwise indicated, the term “include” has the same meaning as “include, but are not limited to,” the term “includes” has the same meaning as “includes, but is not limited to,” and the term “including” has the same meaning as “including, but not limited to.” Similarly, the term “such as” has the same meaning as the term “such as, but not limited to.” Also, terms such as “element” or “component” encompass both elements and components comprising one unit and elements and components that comprise more than one subunit unless specifically stated otherwise.

As utilized in accordance with the present disclosure, the following terms, unless otherwise indicated, shall be understood to have the following meanings:

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “native sequence” polypeptide comprises a polypeptide having the same amino acid sequence as a polypeptide found in nature. Thus, a native sequence polypeptide can have the amino acid sequence of naturally occurring polypeptide from any mammal Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiment, a variant will have at least about 90% amino acid sequence identity. In some embodiment, a variant will have at least about 95% amino acid sequence identity with the native sequence polypeptide. In some embodiment, a variant will have at least about 97% amino acid sequence identity with the native sequence polypeptide.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “FAM150A” includes any native FAM150A from any vertebrate source, including mammals such as primates (e.g. humans) and rodents (e.g., mice and rats), unless otherwise indicated. The term includes full-length, unprocessed FAM150A as well as any form of FAM150A that results from processing in the cell or any fragment thereof that retains the ability to specifically bind LTK and/or ALK with an affinity (Kd) of less than ≦1 μM, ≦100 nM, or ≦10 nM. The term also encompasses naturally occurring variants of FAM150A, e.g., splice variants or allelic variants. In some embodiments, FAM150A is a human FAM150A with an amino acid sequence of SEQ ID NO: 1 (precursor, with signal peptide) or SEQ ID NO: 2 (mature, without signal peptide). A nonlimiting exemplary non-human FAM150A is mouse FAM150A with an amino acid sequence of SEQ ID NO: 32 (precursor, with signal peptide) or SEQ ID NO: 33 (mature, without signal peptide).

The term “FAM150A” also includes full-length FAM150A, FAM150A fragments, and FAM150A variants, with or without a signal peptide. The term “full-length FAM150A”, as used herein, refers to full-length, unprocessed FAM150A as well as any form of FAM150A that results from processing in the cell or any fragment thereof that retains the ability to specifically bind LTK and/or ALK with an affinity (Kd) of less than ≦1 μM, ≦100 nM, or ≦10 nM. In some embodiments, a full-length human FAM150A has the amino acid sequence of SEQ ID NO: 1 (precursor, with signal peptide) or SEQ ID NO: 2 (mature, without signal peptide). As used herein, the term “FAM150A fragment” refers to FAM150A having one or more residues deleted from the N- and/or C-terminus of the full-length FAM150A and that retains the ability to bind LTK and/or ALK. The FAM150A fragment may or may not include an N-terminal signal peptide. As used herein, the term “FAM150A variant” refers to FAM150A that contains amino acid additions, deletions, and substitutions and that remain capable of binding to LTK and/or ALK. Such variants may be at least 80%, 85%, 90%, 92%, 95%, 97%, 98%, or 99% identical to the parent FAM150A. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

As used herein, the term “FAM150A agent” refers collectively to FAM150A and FAM150A fusion molecules, as defined herein.

The term “FAM150B” includes any native FAM150B from any vertebrate source, including mammals such as primates (e.g. humans) and rodents (e.g., mice and rats), unless otherwise indicated. The term includes full-length, unprocessed FAM150B as well as any form of FAM150B that results from processing in the cell or any fragment thereof that retains the ability to specifically bind LTK and/or ALK with an affinity (Kd) of less than ≦1 μM, ≦100 nM, or ≦10 nM. The term also encompasses naturally occurring variants of FAM150B, e.g., splice variants or allelic variants. In some embodiments, FAM150B is a human FAM150B with an amino acid sequence of SEQ ID NO: 3 (precursor, with signal peptide) or SEQ ID NO: 4 (mature, without signal peptide). A nonlimiting exemplary non-human FAM150B is mouse FAM150B with an amino acid sequence of SEQ ID NO: 5 (precursor, with signal peptide) or SEQ ID NO: 6 (mature, without signal peptide).

The term “FAM150B” also includes full-length FAM150B, FAM150B fragments, and FAM150B variants, with or without a signal peptide. The term “full-length FAM150B”, as used herein, refers to full-length, unprocessed FAM150B as well as any form of FAM150B that results from processing in the cell or any fragment thereof that retains the ability to specifically bind LTK and/or ALK with an affinity (Kd) of less than ≦1 μM, ≦100 nM, or ≦10 nM. In some embodiments, a full-length human FAM150B has the amino acid sequence of SEQ ID NO: 3 (precursor, with signal peptide) or SEQ ID NO: 4 (mature, without signal peptide). As used herein, the term “FAM150B fragment” refers to FAM150B having one or more residues deleted from the N- and/or C-terminus of the full-length FAM150B and that retains the ability to bind LTK and/or ALK. The FAM150B fragment may or may not include an N-terminal signal peptide. As used herein, the term “FAM150B variant” refers to FAM150B that contains amino acid additions, deletions, and substitutions and that remain capable of binding to LTK and/or ALK. Such variants may be at least 80%, 85%, 90%, 92%, 95%, 97%, 98%, or 99% identical to the parent FAM150B. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

As used herein, the term “FAM150B agent” refers collectively to FAM150B and FAM150B fusion molecules, as defined herein.

The terms “leukocyte tyrosine kinase receptor” and “LTK” refer herein to any native LTK from any vertebrate source, including mammals such as primates (e.g. humans) and rodents (e.g., mice and rats), unless otherwise indicated. The term includes full-length, unprocessed LTK as well as any form of LTK that results from processing in the cell or any fragment thereof that retains the ability to specifically bind FAM150A and/or FAM150B with an affinity (Kd) of less than ≦1 μM, ≦100 nM, or ≦10 nM. The term also encompasses naturally occurring variants of LTK, e.g., splice variants or allelic variants. In some embodiments, LTK is a human LTK with an amino acid sequence of SEQ ID NO: 7 (precursor, with signal peptide) or SEQ ID NO: 8 (mature, without signal peptide). A nonlimiting exemplary human isoform of LTK is isoform 2, which has the amino acid sequence of SEQ ID NO: 9 (precursor, with signal peptide) or SEQ ID NO: 10 (mature, without signal peptide). A nonlimiting exemplary non-human LTK is mouse LTK, which has the amino acid sequence of SEQ ID NO: 11 (precursor, with signal peptide) or SEQ ID NO: 12 (mature, without signal peptide).

The terms “anaplastic lymphoma kinase receptor” and “ALK” refer herein to any native ALK from any vertebrate source, including mammals such as primates (e.g. humans) and rodents (e.g., mice and rats), unless otherwise indicated. The term includes full-length, unprocessed ALK as well as any form of ALK that results from processing in the cell or any fragment thereof that retains the ability to specifically bind ligand, such as FAM150A and/or FAM150B, with an affinity (Kd) of less than ≦1 μM, ≦100 nM, or ≦10 nM. The term also encompasses naturally occurring variants of ALK, e.g., splice variants or allelic variants. In some embodiments, ALK is a human ALK with an amino acid sequence of SEQ ID NO: 20 (precursor, with signal peptide) or SEQ ID NO: 21 (mature, without signal peptide).

The term “FAM150A activity” or “biological activity” of a FAM150A agent, as used herein, includes any biological effect of FAM150A. In some embodiments, FAM150A activity includes the ability of a FAM150A agent to interact or bind to a substrate or receptor. In some embodiments, FAM150A activity is the ability of a FAM150A agent to stimulate LTK phosphorylation. In some embodiments, FAM150A activity is the ability of a FAM150A agent to induce (e.g., increase) neuronal differentiation. In some embodiments, FAM150A activity includes any biological activity resulting from FAM150A mediated signaling.

The term “FAM150B activity” or “biological activity” of a FAM150B agent, as used herein, includes any biological effect of FAM150B. In some embodiments, FAM150B activity includes the ability of a FAM150B agent to interact or bind to a substrate or receptor. In some embodiments, FAM150B activity is the ability of a FAM150B agent to stimulate LTK phosphorylation. In some embodiments, FAM150B activity includes any biological activity resulting from FAM150B mediated signaling.

The term “antagonist” is used in the broadest sense, and includes any molecule that partially or fully inhibits or neutralizes a biological activity of a polypeptide, such as FAM150A, or that partially or fully inhibits the transcription or translation of a nucleic acid encoding the polypeptide. Exemplary antagonist molecules include, but are not limited to, antagonist antibodies, polypeptide fragments, oligopeptides, organic molecules (including small molecules), aptamers, and antisense nucleic acids. In some embodiments, an antagonist agent may be referred to as a blocking agent (such as a blocking antibody).

The term “FAM150 antagonist” as used herein, encompasses FAM150A antagonists, FAM150B antagonists, and FAM150A/B antagonists, as defined below.

The term “FAM150A antagonist” refers to a molecule that interacts with FAM150A, ALK, and/or LTK, and inhibits FAM150A-mediated signaling. Exemplary FAM150A antagonists include antibodies that bind FAM150A, antibodies that bind LTK, antibodies that bind ALK, LTK extracellular domains (ECDs), and LTK ECD fusion molecules. In some embodiments, a FAM150A antagonist is an antibody to FAM150A. In some embodiments, an FAM150A antagonist blocks binding of FAM150A to LTK. In some embodiments, an FAM150A antagonist blocks binding of FAM150A to ALK.

The term “FAM150B antagonist” refers to a molecule that interacts with FAM150B, ALK, and/or LTK, and inhibits FAM150B-mediated signaling. Exemplary FAM150B antagonists include antibodies that bind FAM150B, antibodies that bind LTK, antibodies that bind ALK, LTK extracellular domains (ECDs), and LTK ECD fusion molecules. In some embodiments, a FAM150B antagonist is an antibody to FAM150B. In some embodiments, a FAM150B antagonist blocks binding of FAM150B to LTK. In some embodiments, a FAM150B antagonist blocks binding of FAM150B to ALK.

The term “FAM150A/B antagonist” refers to a molecule that interacts with FAM150A and FAM150B, or LTK and/or ALK, and inhibits FAM150A- and FAM150B-mediated signaling through LTK and/or ALK. Exemplary FAM150A/B antagonists include antibodies that bind both FAM150A and FAM150B, antibodies that bind LTK, antibodies that bind ALK, LTK extracellular domains (ECDs), and LTK ECD fusion molecules. In some embodiments, a FAM150A/B antagonist is an antibody that binds FAM150A and FAM150B. In some embodiments, a FAM150A/B antagonist blocks binding of both FAM150A and FAM150B to LTK. In some embodiments, a FAM150A/B antagonist is an antibody that binds to LTK and blocks binding of both FAM150A and FAM150B to LTK. In some embodiments, a FAM150A/B antagonist is an antibody that binds to ALK and blocks binding of both FAM150A and FAM150B to ALK. In some embodiments, a FAM150A/B antagonist is an antibody that binds both LTK and ALK and blocks binding of both FAM150A and FAM150B to both receptors.

A FAM150A antagonist or a FAM150A/B antagonist is considered to “block binding of FAM150A to LTK” when it reduces the amount of detectable binding of FAM150A to LTK by at least 50%. In some embodiments, a FAM150A antagonist or FAM150A/B antagonist reduces the amount of detectable binding of FAM150A to LTK by at least 60%, at least 70%, at least 80%, or at least 90%. In some such embodiments, the antagonist is said to block ligand binding by at least 50%, at least 60%, at least 70%, etc.

A FAM150A antagonist or a FAM150A/B antagonist is considered to “block binding of FAM150A to ALK” when it reduces the amount of detectable binding of FAM150A to ALK by at least 50%. In some embodiments, a FAM150A antagonist or FAM150A/B antagonist reduces the amount of detectable binding of FAM150A to ALK by at least 60%, at least 70%, at least 80%, or at least 90%. In some such embodiments, the antagonist is said to block ligand binding by at least 50%, at least 60%, at least 70%, etc.

A FAM150B antagonist or a FAM150A/B antagonist is considered to “block binding of FAM150B to LTK” when it reduces the amount of detectable binding of FAM150B to LTK by at least 50%. In some embodiments, a FAM150B antagonist or FAM150A/B antagonist reduces the amount of detectable binding of FAM150B to LTK by at least 60%, at least 70%, at least 80%, or at least 90%. In some such embodiments, the antagonist is said to block ligand binding by at least 50%, at least 60%, at least 70%, etc.

A FAM150B antagonist or a FAM150A/B antagonist is considered to “block binding of FAM150B to ALK” when it reduces the amount of detectable binding of FAM150B to ALK by at least 50%. In some embodiments, a FAM150B antagonist or FAM150A/B antagonist reduces the amount of detectable binding of FAM150B to ALK by at least 60%, at least 70%, at least 80%, or at least 90%. In some such embodiments, the antagonist is said to block ligand binding by at least 50%, at least 60%, at least 70%, etc.

The terms “inhibition” or “inhibit” refer to a decrease or cessation of any phenotypic characteristic or to the decrease or cessation in the incidence, degree, or likelihood of that characteristic. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

In some embodiments, a FAM150A antagonist or a FAM150A/B antagonist is considered to “inhibit FAM150A-mediated signaling” when it reduces phosphorylation of LTK in the presence of FAM150A. In some embodiments, A FAM150A antagonist or FAM150A/B antagonist reduces phosphorylation of LTK in the presence of FAM150A by at least 60%, at least 70%, at least 80%, or at least 90%.

In some embodiments, a FAM150B antagonist or a FAM150A/B antagonist is considered to “inhibit FAM150B-mediated signaling” when it reduces phosphorylation of LTK in the presence of FAM150B. In some embodiments, A FAM150B antagonist or FAM150A/B antagonist reduces phosphorylation of LTK in the presence of FAM150B by at least 60%, at least 70%, at least 80%, or at least 90%.

The term “agonist” is used in the broadest sense, and includes any molecule that increases, induces, or stimulates a biological activity of a polypeptide, such as LTK. Exemplary agonist molecules include, but are not limited to, agonist antibodies, polypeptide fragments, oligopeptides, organic molecules (including small molecules), and aptamers. An “LTK agonist” as used herein, refers to any molecule that increases, induces, or stimulates a biological activity of LTK. Nonlimiting exemplary LTK agonists include FAM150A agents, FAM150B agents, and LTK agonist antibodies.

The term “FAM150 antibody” as used herein, encompasses FAM150A antibodies, FAM150B antibodies, and FAM150A/B antibodies, as defined below.

The term “FAM150A antibody” or “antibody that binds FAM150A,” as used herein, refers to an antibody that binds to FAM150A. In some embodiments, a FAM150A antibody inhibits FAM150A-mediated signaling. In some embodiments, a FAM150A antibody blocks binding of FAM150A to LTK. In some embodiments, a FAM150A antibody blocks binding of FAM150A to ALK. In some embodiments, a FAM150A antibody refers to an antibody that is capable of binding FAM150A with sufficient affinity such that the antibody is useful as a diagnostic and/or therapeutic agent in targeting FAM150A. In some embodiments, the extent of binding of an FAM150A antibody to an unrelated, non-FAM150A protein is less than about 10% of the binding of the antibody to FAM150A as measured, e.g., by a radioimmunoassay (RIA). In some embodiments, a FAM150A antibody binds to an epitope of FAM150A that is conserved among FAM150A from different species. In some embodiments, a FAM150A antibody binds to the same epitope as a human or humanized FAM150A antibody that binds human FAM150A. In some embodiments, a FAM150A antibody is a FAM150A/B antibody.

The term “FAM150B antibody” or “antibody that binds FAM150B,” as used herein, refers to an antibody that binds to FAM150B. In some embodiments, a FAM150B antibody inhibits FAM150B-mediated signaling. In some embodiments, a FAM150B antibody blocks binding of FAM150B to LTK. In some embodiments, a FAM150B antibody blocks binding of FAM150B to ALK. In some embodiments, FAM150B antibody refers to an antibody that is capable of binding FAM150B with sufficient affinity such that the antibody is useful as a diagnostic and/or therapeutic agent in targeting FAM150B. In some embodiments, the extent of binding of a FAM150B antibody to an unrelated, non-FAM150B protein is less than about 10% of the binding of the antibody to FAM150B as measured, e.g., by a radioimmunoassay (RIA). In some embodiments, a FAM150B antibody binds to an epitope of FAM150B that is conserved among FAM150B from different species. In some embodiments, a FAM150B antibody binds to the same epitope as a human or humanized FAM150B antibody that binds human FAM150B. In some embodiments, a FAM150B antibody is a FAM150A/B antibody.

The term “FAM150A/B antibody” or “antibody that binds FAM150A and FAM150B,” as used herein, refers to an antibody that binds to both FAM150A and FAM150B. In some embodiments, a FAM150A/B antibody inhibits FAM150A- and FAM150B-mediated signaling. In some embodiments, a FAM150A/B antibody blocks binding of both FAM150A and FAM150B to LTK. In some embodiments, a FAM150A/B antibody blocks binding of both FAM150A and FAM150B to ALK. In some embodiments, FAM150A/B antibody refers to an antibody that is capable of binding FAM150A and FAM150B with sufficient affinity such that the antibody is useful as a diagnostic and/or therapeutic agent in targeting FAM150A and FAM150B. In some embodiments, the extent of binding of a FAM150A/B antibody to an unrelated protein is less than about 10% of the binding of the antibody to FAM150A or FAM150B as measured, e.g., by a radioimmunoassay (RIA). In some embodiments, a FAM150A/B antibody binds to an epitope of FAM150A and/or an epitope of FAM150B that is conserved among different species. In some embodiments, a FAM150A/B antibody binds to the same epitope as a human or humanized FAM150A/B antibody that binds human FAM150A and FAM150B.

The term “LTK antibody” or “antibody that binds LTK,” as used herein, refers to an antibody that binds to LTK. In some embodiments, an LTK antibody inhibits FAM150A- and/or FAM150B-mediated signaling. In some embodiments, an LTK antibody inhibits FAM150A- and FAM150B-mediated signaling. In some embodiments, an LTK antibody blocks binding of FAM150A and/or FAM150B to LTK, as defined above. In some embodiments, an LTK antibody blocks binding of FAM150A and FAM150B to LTK, as defined above. Thus, in some embodiments, an LTK antibody is a FAM150A antagonist, a FAM150B antagonist, and/or a FAM150A/B antagonist. In some embodiments, an LTK antibody stimulates LTK phosphorylation. In some embodiments, an LTK antibody stimulates LTK phosphorylation in the absence of FAM150A and/or FAM150B. An LTK antibody that stimulates LTK phosphorylation in the presence or absence of FAM150A and/or FAM150B may be referred to as an “LTK agonist antibody.”

The term “ALK antibody” or “antibody that binds ALK,” as used herein, refers to an antibody that binds to ALK. In some embodiments, an ALK antibody inhibits FAM150A- and/or FAM150B-mediated signaling. In some embodiments, an ALK antibody inhibits FAM150A- and FAM150B-mediated signaling. In some embodiments, an ALK antibody blocks binding of FAM150A and/or FAM150B to ALK, as defined above. In some embodiments, an ALK antibody blocks binding of FAM150A and FAM150B to ALK, as defined above. Thus, in some embodiments, an ALK antibody is a FAM150A antagonist, a FAM150B antagonist, and/or a FAM150A/B antagonist.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term “antibody” as used herein further refers to a molecule comprising complementarity-determining region (CDR) 1, CDR2, and CDR3 of a heavy chain and CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, and antibodies of various species such as mouse, human, cynomolgus monkey, etc.

In some embodiments, an antibody comprises a heavy chain variable region and a light chain variable region. In some embodiments, an antibody comprises at least one heavy chain comprising a heavy chain variable region and at least a portion of a heavy chain constant region, and at least one light chain comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region” as used herein refers to a region comprising heavy chain CDR1, framework (FR) 2, CDR2, FR3, and CDR3. In some embodiments, a heavy chain variable region also comprises at least a portion of an FR1, which is N-terminal to CDR1, and/or at least a portion of an FR4, which is C-terminal to CDR3.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR) 2, CDR2, FR3, and CDR3. In some embodiments, a light chain variable region also comprises an FR1 and/or an FR4.

The term “light chain constant region” as used herein refers to a region comprising a light chain constant domain, C_{L}. Nonlimiting exemplary light chain constant regions include λ, and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen (e.g., FAM150A, FAM150B, ALK, or LTK). Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of a molecule that is bound by a selective binding agent, such as an antibody or a fragment thereof. The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The term “LTK extracellular domain” (“LTK ECD”) includes full-length LTK ECDs, LTK ECD fragments, and LTK ECD variants, and refers to an LTK polypeptide that lacks the intracellular and transmembrane domains, with or without a signal peptide. In some embodiments, an LTK ECD inhibits FAM150A and/or FAM150B-mediated signaling. In some embodiments, an LTK ECD inhibits FAM150A and FAM150B-mediated signaling. Thus, in some embodiments, an LTK ECD is a FAM150A antagonist, a FAM150B antagonist, and/or a FAM150A/B antagonist. The term “full-length LTK ECD”, as used herein, refers to an LTK ECD that extends to the last amino acid of the extracellular domain, and may or may not include an N-terminal signal peptide, and includes natural splice variants in the extracellular domain. In some embodiments, a full-length human LTK ECD has the amino acid sequence of SEQ ID NO: 13 (with signal peptide) or SEQ ID NO: 14 (without signal peptide). In some embodiments, a full-length human LTK ECD has the amino acid sequence of SEQ ID NO: 30 (with signal peptide) or SEQ ID NO: 31 (without signal peptide). Nonlimiting exemplary LTK ECDs are also described, e.g., in Toyoshima et al., 1993, Proc. Natl. Acad. Sci. USA, 90: 5404-5408. In some embodiments, a full-length mouse LTK ECD has the amino acid sequence of SEQ ID NO: 15 (with signal peptide) or SEQ ID NO: 16 (without signal peptide). As used herein, the term “LTK ECD fragment” refers to an LTK ECD having one or more residues deleted from the N- and/or C-terminus of the full-length ECD and that retains the ability to bind FAM150A and/or FAM150B. The LTK ECD fragment may or may not include an N-terminal signal peptide. As used herein, the term “LTK ECD variants” refers to LTK ECDs that contain amino acid additions, deletions, and substitutions and that remain capable of binding to FAM150A and/or FAM150B. Such variants may be at least 80%, 85%, 90%, 92%, 95%, 97%, 98%, or 99% identical to the parent LTK ECD. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

The term “LTK ECD fusion molecule” refers to a molecule comprising an LTK ECD, and one or more “fusion partners.” In some embodiment, the LTK ECD and the fusion partner are covalently linked (“fused”). If the fusion partner is also a polypeptide (“the fusion partner polypeptide”), the LTK ECD and the fusion partner polypeptide may be part of a continuous amino acid sequence, and the fusion partner polypeptide may be linked to either the N-terminus or the C-terminus of the LTK ECD. In such cases, the LTK ECD and the fusion partner polypeptide may be translated as a single polypeptide from a coding sequence that encodes both the LTK ECD and the fusion partner polypeptide (the “LTK ECD fusion protein”). In some embodiments, the LTK ECD and the fusion partner are covalently linked through other means, such as, for example, a chemical linkage other than a peptide bond. Many known methods of covalently linking polypeptides to other molecules (for example, fusion partners) may be used. In other embodiments, the LTK ECD and the fusion partner may be fused through a “linker,” which is comprised of at least one amino acid or chemical moiety.

In some embodiments, the LTK polypeptide and the fusion partner are noncovalently linked. In some such embodiments, they may be linked, for example, using binding pairs. Exemplary binding pairs include, but are not limited to, biotin and avidin or streptavidin, an antibody and its antigen, etc.

Exemplary fusion partners include, but are not limited to, an immunoglobulin Fc domain, albumin, and polyethylene glycol. The amino acid sequences of nonlimiting exemplary Fc domains are shown in SEQ ID NOs: 17 to 19.

In some embodiments, an LTK ECD amino acid sequence is derived from that of a non-human mammal. In such embodiments, the LTK ECD amino acid sequence may be derived from mammals including, but not limited to, rodents (including mice, rats, hamsters), rabbits, simians, felines, canines, equines, bovines, porcines, ovines, caprines, mammalian laboratory animals, mammalian farm animals, mammalian sport animals, and mammalian pets. LTK ECD fusion molecules incorporating a non-human LTK ECD are termed “non-human LTK ECD fusion molecules.” Similar to the human LTK ECD fusion molecules, non-human fusion molecules may comprise a fusion partner, optional linker, and an LTK ECD. Such non-human fusion molecules may also include a signal peptide. A “non-human LTK ECD fragment” refers to a non-human LTK ECD having one or more residues deleted from the N- and/or C-terminus of the full-length ECD and that retains the ability to bind to FAM150A and/or FAM150B of the non-human animal from which the sequence was derived. A “non-human LTK ECD variant” refers to LTK ECDs that contain amino acid additions, deletions, and substitutions and that remain capable of binding to FAM150A and/or FAM150B from the animal from which the sequence was derived.

The term “FAM150A fusion molecule” refers to a molecule comprising FAM150A, as defined herein, and one or more “fusion partners.” In some embodiment, the FAM150A and the fusion partner are covalently linked (“fused”). If the fusion partner is also a polypeptide (“the fusion partner polypeptide”), the FAM150A and the fusion partner polypeptide may be part of a continuous amino acid sequence, and the fusion partner polypeptide may be linked to either the N-terminus or the C-terminus of the FAM150A. In such cases, the FAM150A and the fusion partner polypeptide may be translated as a single polypeptide from a coding sequence that encodes both the FAM150A and the fusion partner polypeptide (the “FAM150A fusion protein”). In some embodiments, the FAM150A and the fusion partner are covalently linked through other means, such as, for example, a chemical linkage other than a peptide bond. Many known methods of covalently linking polypeptides to other molecules (for example, fusion partners) may be used. In other embodiments, the FAM150A and the fusion partner may be fused through a “linker,” which is comprised of at least one amino acid or chemical moiety.

In some embodiments, the FAM150A and the fusion partner are noncovalently linked. In some such embodiments, they may be linked, for example, using binding pairs. Exemplary binding pairs include, but are not limited to, biotin and avidin or streptavidin, an antibody and its antigen, etc.

Exemplary fusion partners include, but are not limited to, an immunoglobulin Fc domain, albumin, and polyethylene glycol. The amino acid sequences of nonlimiting exemplary Fc domains are shown in SEQ ID NOs: 17 to 19.

In some embodiments, FAM150A amino acid sequence is derived from that of a non-human mammal. In such embodiments, the FAM150A amino acid sequence may be derived from mammals including, but not limited to, rodents (including mice, rats, hamsters), rabbits, simians, felines, canines, equines, bovines, porcines, ovines, caprines, mammalian laboratory animals, mammalian farm animals, mammalian sport animals, and mammalian pets. FAM150A fusion molecules incorporating a non-human FAM150A are termed “non-human FAM150A fusion molecules.” Similar to the human FAM150A fusion molecules, non-human fusion molecules may comprise a fusion partner, optional linker, and a FAM150A. Such non-human fusion molecules may also include a signal peptide. A “non-human FAM150A fragment” refers to a non-human FAM150A having one or more residues deleted from the N- and/or C-terminus of the full-length ECD and that retains the ability to bind to LTK and/or ALK of the non-human animal from which the sequence was derived. A “non-human FAM150A variant” refers to FAM150A that contain amino acid additions, deletions, and substitutions and that remain capable of binding to LTK and/or ALK from the animal from which the sequence was derived.

The term “FAM150B fusion molecule” refers to a molecule comprising FAM150B, as defined herein, and one or more “fusion partners.” In some embodiment, the FAM150B and the fusion partner are covalently linked (“fused”). If the fusion partner is also a polypeptide (“the fusion partner polypeptide”), the FAM150B and the fusion partner polypeptide may be part of a continuous amino acid sequence, and the fusion partner polypeptide may be linked to either the N-terminus or the C-terminus of the FAM150B. In such cases, the FAM150B and the fusion partner polypeptide may be translated as a single polypeptide from a coding sequence that encodes both the FAM150B and the fusion partner polypeptide (the “FAM150B fusion protein”). In some embodiments, the FAM150B and the fusion partner are covalently linked through other means, such as, for example, a chemical linkage other than a peptide bond. Many known methods of covalently linking polypeptides to other molecules (for example, fusion partners) may be used. In other embodiments, the FAM150B and the fusion partner may be fused through a “linker,” which is comprised of at least one amino acid or chemical moiety.

In some embodiments, the FAM150B and the fusion partner are noncovalently linked. In some such embodiments, they may be linked, for example, using binding pairs. Exemplary binding pairs include, but are not limited to, biotin and avidin or streptavidin, an antibody and its antigen, etc.

Exemplary fusion partners include, but are not limited to, an immunoglobulin Fc domain, albumin, and polyethylene glycol. The amino acid sequences of nonlimiting exemplary Fc domains are shown in SEQ ID NOs: 17 to 19.

In some embodiments, FAM150B amino acid sequence is derived from that of a non-human mammal. In such embodiments, the FAM150B amino acid sequence may be derived from mammals including, but not limited to, rodents (including mice, rats, hamsters), rabbits, simians, felines, canines, equines, bovines, porcines, ovines, caprines, mammalian laboratory animals, mammalian farm animals, mammalian sport animals, and mammalian pets. FAM150B fusion molecules incorporating a non-human FAM150B are termed “non-human FAM150B fusion molecules.” Similar to the human FAM150B fusion molecules, non-human fusion molecules may comprise a fusion partner, optional linker, and a FAM150B. Such non-human fusion molecules may also include a signal peptide. A “non-human FAM150B fragment” refers to a non-human FAM150B having one or more residues deleted from the N- and/or C-terminus of the full-length ECD and that retains the ability to bind to LTK and/or ALK of the non-human animal from which the sequence was derived. A “non-human FAM150B variant” refers to FAM150B that contain amino acid additions, deletions, and substitutions and that remain capable of binding to LTK and/or ALK from the animal from which the sequence was derived.

The term “signal peptide” refers to a sequence of amino acid residues located at the N-terminus of a polypeptide that facilitates secretion of a polypeptide from a mammalian cell. A signal peptide may be cleaved upon export of the polypeptide from the mammalian cell, forming a mature protein. Signal peptides may be natural or synthetic, and they may be heterologous or homologous to the protein to which they are attached. Exemplary signal peptides include, but are not limited to, the signal peptides of FAM150A, FAM150B, LTK, and ALK. Exemplary signal peptides also include signal peptides from heterologous proteins. A “signal sequence” refers to a polynucleotide sequence that encodes a signal peptide. In some embodiments, a FAM150A agent lacks a signal peptide. In some embodiments, a FAM150A agent includes at least one signal peptide, which may be a native FAM150A signal peptide or a heterologous signal peptide. In some embodiments, a FAM150B agent lacks a signal peptide. In some embodiments, a FAM150B agent includes at least one signal peptide, which may be a native FAM150B signal peptide or a heterologous signal peptide. In some embodiments, an LTK ECD lacks a signal peptide. In some embodiments, an LTK ECD includes at least one signal peptide, which may be a native LTK signal peptide or a heterologous signal peptide.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate (including, for example, bronchioalveolar lavage fluid and induced sputum); blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A condition “has previously been characterized as having [a characteristic]” when such characteristic of the condition has been shown in at least a subset of patients with the condition, or in one or more animal models of the condition. In some embodiments, such characteristic of the condition does not have to be determined in the patient to be treated with an LTK agonist (such as an LTK agonist antibody, a FAM150A agent, and/or a FAM150B agent), or one or more FAM150 antagonists of the present invention. The presence of the characteristic in a specific patient who is to be treated using the present methods and/or compositions need not have been determined in order for the patient to be considered as having a condition that has previously been characterized as having the characteristic.

A “disorder” or “disease” is any condition that would benefit from treatment with an LTK agonist (such as an LTK agonist antibody, a FAM150A agent, and/or a FAM150B agent), or one or more FAM150 antagonists of the invention. This includes chronic and acute disorders or diseases including those pathological conditions which predispose the mammal to the disorder in question. Nonlimiting examples of disorders to be treated herein include cancers, autoimmune diseases, and neurodegenerative diseases.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

A “chemotherapeutic agent” is a chemical compound useful in the treatment of cancer. Examples of chemotherapeutic agents include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Ill.), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “autoimmune disease” or “autoimmune disorder” refers to a disease or disorder arising from and directed against an individual's own tissues. Examples of autoimmune diseases or disorders include, but are not limited to arthritis (rheumatoid arthritis, juvenile rheumatoid arthritis, osteoarthritis, psoriatic arthritis), psoriasis, dermatitis, polymyositis/dermatomyositis, toxic epidermal necrolysis, systemic scleroderma and sclerosis, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Diamond Blackfan anemia, immune hemolytic anemia including autoimmune hemolytic anemia (MBA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic thrombocytopenic purpura (TTP), autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre' syndrome, large vessel vasculitis (including polymyalgia rheumatica and giant cell (Takayasu's) arteritis), medium vessel vasculitis (including Kawasaki's disease and polyarteritis nodosa), ankylosing spondylitis, Berger's disease (IgA nephropathy), rapidly progressive glomerulonephritis, primary biliary cirrhosis, Celiac sprue (gluten enteropathy), cryoglobulinemia, amyotrophic lateral sclerosis (ALS), coronary artery disease etc.

Examples of “disease-modifying anti-rheumatic drugs” or “DMARDs” include hydroxycloroquine, sulfasalazine, methotrexate, leflunomide, azathioprine, D-penicillamine, gold salts (oral), gold salts (intramuscular), minocycline, cyclosporine including cyclosporine A and topical cyclosporine, staphylococcal protein A, and TNF-inhibitors, including salts, variants, and derivatives thereof, etc. Exemplary DMARDs herein are non-biological DMARDs, including, in particular, azathioprine, chloroquine, hydroxychloroquine, leflunomide, methotrexate and sulfasalazine.

A “TNF inhibitor” herein is an agent that inhibits, to some extent, a biological function of TNF-alpha, generally through binding to TNF-alpha and neutralizing its activity. Examples of TNF inhibitors specifically contemplated herein are etanercept (Enbrel®), infliximab (Remicade®), and adalimumab (Humira®), certolizumab pegol (Cimzia®), and golimumab (Simponi®).

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including Solu-Medrol®. methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine antagonists such as cytokine antibodies or cytokine receptor antibodies including anti-interferon-alpha, -beta, or -gamma antibodies, anti-tumor necrosis factor (TNF)-alpha antibodies (infliximab (Remicade®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002)); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as TIOB9. Some immunosuppressive agents herein are also DMARDs, such as methotrexate. Examples of immunosuppressive agents herein include cyclophosphamide, chlorambucil, azathioprine, leflunomide, MMF, or methotrexate.

The term “neurodegenerative disease” or “neurodegenerative disorder” refers to a disease, disorder or condition of the nervous system (e.g., the central nervous system, CNS) that is characterized by gradual and progressive loss of neural tissue, neurotransmitter, or neural functions. Nonlimiting exemplary neurodegenerative diseases or disorders include Alzheimer's disease, Huntington's disease, Parkinson's disease, Parkinson's-plus diseases, amyotrophic lateral sclerosis (ALS), ischemia, stroke, intracranial hemorrhage, cerebral hemorrhage, trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, multiple system atrophy, progressive supranuclear palsy, corticobasal degeneration, dementia with Lewy bodies, frontotemporal dementia, demyelinating diseases, Guillain-Barre syndrome, multiple sclerosis, Charcot-Marie-Tooth disease, prion disease, Creutzfeldt-Jakob disease, Gerstmann-Straussler-Scheinker syndrome (GSS), fatal familial insomnia (FFI), bovine spongiform encephalopathy, Pick's disease, epilepsy, AIDS demential complex, nerve damage caused by exposure to toxic compounds, heavy metals, industrial solvents, drugs, or chemotherapeutic agents; injury to the nervous system caused by physical, mechanical, or chemical trauma; glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis.

The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. In some embodiments, the term “neurite” refers to both dendrites and axons, or to precursors of dendrites and axons. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column).

“Treatment,” as used herein, covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of an LTK agonist (such as an LTK agonist antibody, a FAM150A agent, and/or a FAM150B agent), or a FAM150 antagonist of the invention may vary according to factors such as the disease state, age, sex, and weight of the individual, and the ability of the antagonist to elicit a desired response in the individual. A therapeutically effective amount encompasses an amount in which any toxic or detrimental effects of an LTK agonist or the FAM150 antagonist are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### Therapeutic Compositions and Methods

Methods of Treating Diseases

FAM150 antagonists (including FAM150A antagonists, FAM150B antagonists, and FAM150A/B antagonists) are provided for use in methods of treating humans and other mammals. Methods of treating a disease comprising administering FAM150 antagonists to humans and other mammals are provided. In addition, LTK agonists (such as an LTK agonist antibodies, FAM150A agents, and FAM150B agents) are provided for use in methods of treating humans and other mammals. Methods of treating a disease comprising administering an LTK agonist (such as an LTK agonist antibody, a FAM150A agent, and/or a FAM150B agent) to humans and other mammals are provided.

### Methods of Treating Autoimmune Disorders

LTK is expressed on T cells and plasmacytoid dendritic cells, e.g., as demonstrated herein. However, prior to the present inventors' identification of the ligand(s) for LTK, blocking LTK-ligand interactions and downstream signaling were difficult. In some embodiments, methods of treating autoimmune conditions are provided, wherein the methods comprise administering a FAM150 antagonist (such as a FAM150A antagonist, a FAM150B antagonist, and/or a FAM150A/B antagonist) to a subject with an autoimmune condition. In some embodiments, use of FAM150 antagonists (such as a FAM150A antagonist, a FAM150B antagonist, and/or a FAM150A/B antagonist) for treating autoimmune conditions are provided. Nonlimiting exemplary autoimmune conditions that may be treated with FAM150 antagonists are provided herein, including systemic lupus erythematosus (SLE), multiple sclerosis, rheumatoid arthritis, and ankylosing spondylitis.

Systemic lupus erythematosus (SLE; also referred to as “lupus”) is an autoimmune disease or disorder that in general involves antibodies that attack connective tissue. Lupus can results in skin lesions, joint pain and swelling, kidney disease (lupus nephritis, an inflammation of the kidneys that occurs in patients with SLE), fluid around the heart and/or lungs, inflammation of the heart, and various other systemic conditions. Specific molecular triggers for systemic lupus erythematosus (SLE) have been difficult to identify, and treatments for SLE have therefore focused on the inflammatory symptoms of lupus rather than the underlying molecular cause. Gain-of-function mutations in leukocyte tyrosine kinase (LTK) were found to correlate with aberrant activation of B1 cells in a mouse model of SLE. See e.g., Li et al., Human Mol. Gen. 13(2): 171-179 (2004). Similar mutations were found in some human patients with SLE. Id. Those results suggest that an increase in signaling through LTK may contribute to the development of SLE in some patients.

In some embodiments, methods of treating systemic lupus erythematosus (SLE) are provided, wherein the methods comprise administering a FAM150 antagonist to a subject with SLE. In some embodiments, methods of treating SLE are provided, wherein the methods comprise administering a FAM150A antagonist, a FAM150B antagonist, and/or a FAM150A/B antagonist to a subject with SLE. In some embodiments, use of a FAM150 antagonist for treating SLE is provided.

Rheumatoid arthritis (RA) is a chronic autoimmune disease characterized primarily by inflammation of the lining (synovium) of the joints, which can lead to joint damage, resulting in chronic pain, loss of function, and disability. Because RA can affect multiple organs of the body, including skin, lungs, and eyes, it is referred to as a systemic illness. In some embodiments, RA is diagnosed according to the 1987, 2000, or 2010 criteria for the classification of RA (American Rheumatism Association or American College of Rheumatology), or any similar criteria.

In some embodiments, methods of treating rheumatoid arthritis (RA) are provided, wherein the methods comprise administering a FAM150 antagonist to a subject with RA. In some embodiments, methods of treating RA are provided, wherein the methods comprise administering a FAM150A antagonist, a FAM150B antagonist, and/or a FAM150A/B antagonist to a subject with RA. In some embodiments, use of a FAM150 antagonist for treating RA is provided.

Multiple sclerosis (MS) is a chronic and often disabling disease of the central nervous system characterized by the progressive destruction of the myelin. Demyelination occurs when the myelin sheath becomes inflamed, injured, and detaches from the nerve fiber. There are four internationally recognized forms of MS, namely, primary progressive multiple sclerosis (PPMS), relapsing-remitting multiple sclerosis (RRMS), secondary progressive multiple sclerosis (SPMS), and progressive relapsing multiple sclerosis (PRMS).

In some embodiments, methods of treating multiple sclerosis (MS) are provided, wherein the methods comprise administering a FAM150 antagonist to a subject with MS. In some embodiments, methods of treating MS are provided, wherein the methods comprise administering a FAM150A antagonist, a FAM150B antagonist, and/or a FAM150A/B antagonist to a subject with MS. In some embodiments, use of a FAM150 antagonist for treating MS is provided.

Ankylosing spondylitis (AS) is an inflammatory condition that causes some of the vertebrae in the spine to fuse together, resulting in a loss of flexibility and, in some instances, a hunched posture.

In some embodiments, methods of treating ankylosing spondylitis (AS) are provided, wherein the methods comprise administering a FAM150 antagonist to a subject with AS. In some embodiments, methods of treating AS are provided, wherein the methods comprise administering a FAM150A antagonist, a FAM150B antagonist, and/or a FAM150A/B antagonist to a subject with AS. In some embodiments, use of a FAM150 antagonist for treating AS is provided.

In some embodiments, the FAM150A antagonist is selected from a FAM150A antibody, an LTK antibody, an LTK ECD, an LTK ECD fusion molecule, and an ALK antibody. In some embodiments, the FAM150B antagonist is selected from a FAM150B antibody, an LTK antibody, an LTK ECD, an LTK ECD fusion molecule, and an ALK antibody. In some embodiments, the FAM150A/B antagonist is selected from a FAM150A/B antibody, an LTK antibody, an LTK ECD, an LTK ECD fusion molecule, and an ALK antibody. In some embodiments, the FAM150 antagonist is selected from a FAM150A antibody, a FAM150B antibody, and a FAM150A/B antibody.

### Methods of Treating Cancer

In some embodiments, methods for treating or preventing a cancer associated with increased expression and/or activity of FAM150A, FAM150B and/or LTK are provided, the methods comprising administering an effective amount of FAM150 antagonist to a subject in need of such treatment.

LTK and/or its ligands are expressed in various cancer types. Further, LTK has been reported to be overexpressed in human leukemias. See, e.g., Roll et al., 2012, PLoS ONE, 7: e31733. Again, however, prior to the present inventors' identification of the ligand(s) for LTK, blocking LTK-ligand interactions and downstream signaling were difficult. In some embodiments, methods of treating cancer are provided, wherein the methods comprise administering a FAM150 antagonist to a subject with cancer. In some embodiments, methods of treating cancer are provided, wherein the methods comprise administering a FAM150A antagonist, a FAM150B antagonist, and/or a FAM150A/B antagonist to a subject with cancer. In some embodiments, use of a FAM150 antagonist for treating cancer is provided. Nonlimiting exemplary cancers that may be treated with FAM150 antagonists are provided herein, including lung cancer, leukemia, breast cancer, ovarian cancer, kidney cancer, colon cancer, and bladder cancer. In some embodiments, lung cancer is non-small cell lung cancer or lung squamous cell carcinoma. In some embodiments, leukemia is acute myeloid leukemia or chronic lymphocytic leukemia. In some embodiments, breast cancer is breast invasive carcinoma. In some embodiments, ovarian cancer is ovarian serous cystadenocarcinoma. In some embodiments, kidney cancer is kidney renal clear cell carcinoma. In some embodiments, colon cancer is colon adenocarcinoma. In some embodiments, bladder cancer is bladder urothelial carcinoma.

In some embodiments, methods of treating lung cancer are provided, wherein the methods comprise administering a FAM150 antagonist to a subject with lung cancer. In some embodiments, methods of treating lung cancer are provided, wherein the methods comprise administering a FAM150A antagonist, a FAM150B antagonist, and/or a FAM150A/B antagonist to a subject with lung cancer. In some embodiments, use of a FAM150 antagonist for treating lung cancer is provided. Lung cancer includes, but is not limited to, both small cell lung cancer and non-small cell lung cancers. Non-small cell lung cancer includes, but is not limited to, squamous cell lung cancer, adenocarcinoma, large-cell lung carcinoma, sarcomatoid carcinoma, carcinoid tumors, pulmonary pleomorphic carcinoma, and adenosquamous carcinoma and bronchioloalveolar carcinoma. Small cell lung cancer may, in some embodiments, be referred to as “oat-cell” cancer, and includes, but is not limited to, combined small-cell carcinoma, which comprises a mixture of small cell and non-small cell carcinomas. In some embodiments, the cancer is non-small cell lung cancer or lung squamous cell carcinoma.

In some embodiments, methods of treating acute myeloid leukemia (AML) are provided, wherein the methods comprise administering a FAM150 antagonist to a subject with AML. In some embodiments, methods of treating AML are provided, wherein the methods comprise administering a FAM150A antagonist, a FAM150B antagonist, and/or a FAM150A/B antagonist to a subject with AML. In some embodiments, use of a FAM150 antagonist for treating AML is provided.

In some embodiments, methods of treating chronic lymphocytic leukemia (CLL) are provided, wherein the methods comprise administering a FAM150 antagonist to a subject with CLL. In some embodiments, methods of treating CLL are provided, wherein the methods comprise administering a FAM150A antagonist, a FAM150B antagonist, and/or a FAM150A/B antagonist to a subject with CLL. In some embodiments, use of a FAM150 antagonist for treating CLL is provided.

In some embodiments, methods of treating a cancer selected from lung cancer, leukemia, breast cancer, ovarian cancer, kidney cancer, colon cancer, and bladder cancer are provided, wherein the methods comprise administering a FAM150 antagonist to a subject with the cancer. In some embodiments, methods of treating a cancer selected from lung cancer, leukemia, breast cancer, ovarian cancer, kidney cancer, colon cancer, and bladder cancer are provided, wherein the methods comprise administering a FAM150A antagonist, a FAM150B antagonist, and/or a FAM150A/B antagonist to a subject with the cancer. In some embodiments, use of a FAM150 antagonist for treating a cancer selected from lung cancer, leukemia, breast cancer, ovarian cancer, kidney cancer, colon cancer, and bladder cancer is provided. In some embodiments, lung cancer is non-small cell lung cancer or lung squamous cell carcinoma. In some embodiments, leukemia is acute myeloid leukemia or chronic lymphocytic leukemia. In some embodiments, breast cancer is breast invasive carcinoma. In some embodiments, ovarian cancer is ovarian serous cystadenocarcinoma. In some embodiments, kidney cancer is kidney renal clear cell carcinoma. In some embodiments, colon cancer is colon adenocarcinoma. In some embodiments, bladder cancer is bladder urothelial carcinoma.

In some embodiments, the FAM150A antagonist is selected from a FAM150A antibody, an LTK antibody, an LTK ECD, an LTK ECD fusion molecule, and an ALK antibody. In some embodiments, the FAM150B antagonist is selected from a FAM150B antibody, an LTK antibody, an LTK ECD, an LTK ECD fusion molecule, and an ALK antibody. In some embodiments, the FAM150A/B antagonist is selected from a FAM150A/B antibody, an LTK antibody, an LTK ECD, an LTK ECD fusion molecule, and an ALK antibody. In some embodiments, the FAM150 antagonist is selected from a FAM150A antibody, a FAM150B antibody, and a FAM150A/B antibody.

### Methods of Treating Neurodegenerative Diseases

As demonstrated herein, stimulation of LTK signaling with FAM150A in PC12 cells leads to neurite outgrowth and differentiation. This result confirms previous reports using an LTK-CSF1R fusion. See, e.g., Yamada et al., 2008, Mol. Neurosc., 19: 1733-1738. A possible role for LTK in adult neurogenesis has also previously been described. See, e.g., Weiss et al., 2012, Pharmacol. Biochem. Behav. 100: 566-574. Prior to the present inventors' identification of the ligand(s) for LTK, it would not have been possible to increase levels of LTK ligand(s) to increase LTK signaling in neural cells. In some embodiments, methods of treating neurodegenerative diseases are provided, wherein the methods comprise administering an LTK agonist (such as an LTK agonist antibody, a FAM150A agent, and/or a FAM150B agent) to a subject with a neurodegenerative disease.

Nonlimiting exemplary neurodegenerative diseases are provided herein, including Parkinson's disease, Huntington's disease, and Alzheimer's disease. Parkinson's disease is a disorder of the brain that leads to shaking and difficulty with walking, movement, and coordination. In at least some instances of Parkinson's disease, the nerve cells in the brain that make dopamine slowly degenerate. Huntington's disease is typically a genetic disorder in which nerve cells in certain parts of the brain degenerate. In some embodiments, Alzheimer's disease is a degenerative brain disorder that is characterized by formation of neurofibrillary tangles (tangled protein fragments in nerve cells) and neuritic plaques (extracellular deposits of amyloid in the brain).

In some embodiments, methods of treating Parkinson's disease are provided, wherein the methods comprise administering an LTK agonist (such as an LTK agonist antibody, a FAM150A agent, and/or a FAM150B agent) to a subject with Parkinson's disease. In some embodiments, methods of treating Huntington's disease are provided, wherein the methods comprise administering an LTK agonist (such as an LTK agonist antibody, a FAM150A agent, and/or a FAM150B agent) to a subject with Huntington's disease. In some embodiments, methods of treating Alzheimer's disease are provided, wherein the methods comprise administering an LTK agonist (such as an LTK agonist antibody, a FAM150A agent, and/or a FAM150B agent) to a subject with Alzheimer's disease.

In some embodiments, a molecule administered in the methods is FAM150A and/or FAM150B. In some embodiments, a molecule administered in the methods is a FAM150A fusion molecule and/or FAM150B fusion molecule. Nonlimiting exemplary fusion partners that may be used in a FAM150A fusion molecule and/or FAM150B fusion molecule are described herein. In some embodiments, a FAM150A fusion molecule comprises FAM150A fused to an Fc. In some embodiments, a FAM150B fusion molecule comprises FAM150B fused to an Fc. In some embodiments, a molecule administered in the methods is an LTK agonist antibody.

As noted above, in some embodiments, “treating” a disease comprises alleviating one or more symptoms of the disease, either temporarily or permanently. In some embodiments, long-term alleviation of symptoms occurs with regular dosing of an LTK agonist (such as an LTK agonist antibody, a FAM150A agent, and/or a FAM150B agent), or a FAM150 antagonist. Cessation of the treatment, in some embodiments, may result in a resumption of one or more symptoms of the disease.

Routes of Administration and Carriers

In various embodiments, LTK agonists (such as LTK agonist antibodies, FAM150A agents, and FAM150B agents), or FAM150 antagonists may be administered subcutaneously or intravenously. In some embodiments, LTK agonists or a FAM150 antagonist may be administered in vivo by various routes, including, but not limited to, oral, intra-arterial, parenteral, intranasal, intramuscular, intracardiac, intraventricular, intratracheal, buccal, rectal, intraperitoneal, by inhalation, intradermal, topical, transdermal, and intrathecal, or otherwise, e.g., by implantation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. In some embodiments, an LTK agonist or a FAM150 antagonist is delivered using gene therapy. As a nonlimiting example, a nucleic acid molecule encoding an LTK agonist or a FAM150 antagonist may be coated onto gold microparticles and delivered intradermally by a particle bombardment device, or “gene gun,” e.g., as described in the literature (see, e.g., Tang et al., Nature 356:152-154 (1992)).

In various embodiments, compositions comprising an LTK agonist (such as an LTK agonist antibody, a FAM150A agent, and/or a FAM150B agent), or a FAM150 antagonist are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising an LTK agonist (such as an LTK agonist antibody, a FAM150A agent, and/or a FAM150B agent), or a FAM150 antagonist may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A nonlimiting exemplary biodegradable formulation includes poly lactic acid-glycolic acid polymer. A nonlimiting exemplary non-biodegradable formulation includes a polyglycerin fatty acid ester. Certain methods of making such formulations are described, for example, in EP 1 125 584 A1.

Pharmaceutical dosage packs comprising one or more containers, each containing one or more doses of an LTK agonist (such as an LTK agonist antibody, a FAM150A agent, and/or a FAM150B agent), or a FAM150 antagonist, are also provided. In some embodiments, a unit dosage is provided wherein the unit dosage contains a predetermined amount of a composition comprising an LTK agonist or a FAM150 antagonist, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In some embodiments, an LTK agonist (such as an LTK agonist antibody, a FAM150A agent, and/or a FAM150B agent), or a FAM150 antagonist may be administered in an amount in the range of about 50 μg/kg body weight to about 50 mg/kg body weight per dose. In some embodiments, an LTK agonist or a FAM150 antagonist may be administered in an amount in the range of about 100 μg/kg body weight to about 50 mg/kg body weight per dose. In some embodiments, an LTK agonist or a FAM150 antagonist may be administered in an amount in the range of about 100 μg/kg body weight to about 20 mg/kg body weight per dose. In some embodiments, an LTK agonist or a FAM150 antagonist may be administered in an amount in the range of about 0.5 mg/kg body weight to about 20 mg/kg body weight per dose.

In some embodiments, an LTK agonist (such as an LTK agonist antibody, a FAM150A agent, and/or a FAM150B agent), or a FAM150 antagonist may be administered in an amount in the range of about 10 mg to about 1,000 mg per dose. In some embodiments, an LTK agonist or a FAM150 antagonist may be administered in an amount in the range of about 20 mg to about 500 mg per dose. In some embodiments, an LTK agonist or a FAM150 antagonist may be administered in an amount in the range of about 20 mg to about 300 mg per dose. In some embodiments, an LTK agonist or a FAM150 antagonist may be administered in an amount in the range of about 20 mg to about 200 mg per dose.

The LTK agonist or FAM150 antagonist compositions may be administered as needed to subjects. In some embodiments, an effective dose of an LTK agonist or a FAM150 antagonist is administered to a subject one or more times. In various embodiments, an effective dose of an LTK agonist or a FAM150 antagonist is administered to the subject once a month, less than once a month, such as, for example, every two months, every three months, or every six months. In other embodiments, an effective dose of an LTK agonist or a FAM150 antagonist is administered more than once a month, such as, for example, every two weeks, every week, twice per week, three times per week, daily, or multiple times per day. An effective dose of an LTK agonist or a FAM150 antagonist is administered to the subject at least once. In some embodiments, the effective dose of an LTK agonist or a FAM150 antagonist may be administered multiple times, including for periods of at least a month, at least six months, or at least a year. In some embodiments, an LTK agonist or a FAM150 antagonist is administered to a subject as-needed to alleviate one or more symptoms of a condition.

Combination Therapy

An LTK agonist or a FAM150 antagonist according to the invention, including any functional fragments thereof, may be administered to a subject in need thereof in combination with other biologically active substances or other treatment procedures for the treatment of diseases. For example, LTK agonists (such as an LTK agonist antibodies, FAM150A agents, and FAM150B agents) or FAM150 antagonists may be administered alone or with other modes of treatment. In some embodiments, a FAM150A agent and a FAM150B agent may be administered together, or more than one FAM150 antagonist may be administered. They may be provided before, substantially contemporaneous with, or after other modes of treatment, such as radiation therapy.

For treatment of systemic lupus erythematosus (SLE), FAM150 antagonists may be administered with other therapeutic agents, for example, hydroxychloroquine (Plaquenil®); nonsteroidal anti-inflammatory drugs (NSAIDs), including, but not limited to, ibuprofen, naproxen sodium, aspirin, and sulindac; corticosteroids, such as prednisone, methylprednisone, and prednisolone; immunosuppressants, such as cyclosporine, chlorambucil, cyclophosphamide (Cytoxan®), azathioprine (Imuran®, Azasan®), mycophenolate (Cellcept®), leflunomide (Arava®), methotrexate (Trexall™), and belimumab (Benlysta®); and other drugs, such as mycophenolate mofetil and rituximab) (Rituxan®).

For treatment of multiple sclerosis (MS), FAM150 antagonists may be administered with other therapeutic agents, for example, interferon alpha; interferon beta; prednisone; anti-alpha4 integrin antibodies such as Tysabri®; anti-CD20 antibodies such as Rituxan®; FTY720 (fingolimod; Gilenya®); and cladribine (Leustatin®).

For treatment of ankylosing spondylitis (AS), FAM150 antagonists may be administered with other therapeutic agents, such as nonsteroidal anti-inflammatory drugs (NSAIDs), including, but not limited to, ibuprofen, naproxen sodium, aspirin, and sulindac; and TNF inhibitors, such as adalimumab (Humira®), etanercept (Enbrel®), infliximab (Remicade®), and golimumab (Simponi®).

For treatment of rheumatoid arthritis (RA), FAM150 antagonists may be administered with other therapeutic agents, for example, methotrexate, anti-TNF agents, including anti-TNF antibodies such as Remicade® (infliximab), Humira® (adalimumab), Simponi® (golimumab), and certolizumab pegol, and soluble TNF receptors, such as Enbrel (etanercept); glucocorticoids such as prednisone; leflunomide; azathioprine; JAK inhibitors such as CP 590690; SYK inhibitors such as R788; anti-IL-6 agents, including anti-IL-6 antibodies such as elsilimomab, siltuximab, and sirukumab, and anti-IL-6R antibodies such as Actemra® (tocilizumab); anti-CD-20 agents, including anti-CD20 antibodies such as Rituxan® (rituximab), ibritumomab tiuxetan, ofatumumab, ocrelizumab, veltuzumab, and tositumomab; anti-CD19 agents, such as anti-CD19 antibodies; anti-GM-CSF agents, such as anti-GM-CSF antibodies and anti-GM-CSFR antibodies; anti-IL-1 agents, such as IL-1 receptor antagonists, including anakinra; CTLA-4 agonists, such as CTLA4-Ig fusions, including abatacept and belatacept; immunosuppressants such as cyclosporine.

For treatment of cancer, the methods may further comprise administering an effective amount of one or more FAM150 antagonists to a subject in need of such treatment. In some embodiments, the FAM150 antagonist is administered in conjunction with one or more of anti-cancer agents, such as the chemotherapeutic agent, growth inhibitory agent, anti-angiogenesis agent or anti-neoplastic composition. Nonlimiting examples of chemotherapeutic agent, growth inhibitory agent, anti-angiogenesis agent and anti-neoplastic composition that can be used in combination with one or more FAM150 antagonists of the present invention are provided herein under “Definitions.”

For treatment of a neurodegenerative disorder, in some embodiments, the methods may further comprise administering a therapeutic agent selected from cholinesterase inhibitors, such as donepezil (Aricept®), galantamine (Razadyne®), and rivastigmine (Exelon®); memantine (Namenda®); tetrabenazine (Xenazine®), antipsychotic agents, such as haloperidol (Haldol®) and clozapine, clonazepam (Klonapin®), and diazepam; antidepressants, such as escitalopram (Lexapro®), fluoxetine (Prozac®, Sarafem®) and sertraline (Zoloft®); anti-psychotic agents, such as lithium (Lithobid®); and anticonvulsants, such as valproic acid (Depakene®), divalproex (Depakote®), and lamotrigine (Lamictal®); carbidopa-levodopa (Parcopa®); dopamine agonists, such as pramipexole (Mirapex®), ropinirole (Requip®), and apomorphine (Apokyn®); monoamine oxidase B inhibitors, such as selegiline (Eldepryl®, Zelapar®) and rasagiline (Azilect®); catechol O-methyltransferase (COMT) inhibitors, such as entacapone (Comtan®) and tolcapone (Tasmar®); anticholinergics, such as benztropine (Cogentin®) and trihexyphenidyl; and amantadine.

For treatment of Huntington's disease, an LTK agonist (such as an LTK agonist antibody, a FAM150A agent, and/or a FAM150B agent) may be administered with other therapeutic agents, such as agents to treat movement disorders, including tetrabenazine (Xenazine®), antipsychotic agents, such as haloperidol (Haldol®) and clozapine, clonazepam (Klonapin®), and diazepam; antidepressants, such as escitalopram) (Lexapro®), fluoxetine (Prozac®, Sarafem®) and sertraline (Zoloft®); anti-psychotic agents, such as lithium (Lithobid®); and anticonvulsants, such as valproic acid (Depakene®), divalproex (Depakote®), and lamotrigine (Lamictal®).

For treatment of Parkinson's disease, an LTK agonist (such as an LTK agonist antibody, a FAM150A agent, and/or a FAM150B agent) may be administered with other therapeutic agents, such as carbidopa-levodopa (Parcopa®); dopamine agonists, such as pramipexole (Mirapex®), ropinirole (Requip®), and apomorphine (Apokyn®); monoamine oxidase B inhibitors, such as selegiline (Eldepryl®, Zelapar®) and rasagiline (Azilect®); catechol O-methyltransferase (COMT) inhibitors, such as entacapone (Comtan®) and tolcapone (Tasmar®); anticholinergics, such as benztropine (Cogentin®) and trihexyphenidyl; and amantadine.

For treatment of Alzheimer's disease, an LTK agonist (such as an LTK agonist antibody, a FAM150A agent, and/or a FAM150B agent) may be administered with other therapeutic agents, such as cholinesterase inhibitors, including donepezil (Aricept®), galantamine (Razadyne®), and rivastigmine (Exelon®); and memantine (Namenda®).

### FAM150 Antibodies, LTK Antibodies, and ALK Antibodies

In some embodiments, antibodies that block binding of FAM150A and/or FAM150B to LTK are provided. In some embodiments, antibodies that inhibit FAM150A-mediated signaling are provided. In some such embodiments, the antibody is a FAM150A antibody. In some embodiments, antibodies that inhibit FAM150B-mediated signaling are provided. In some such embodiments, the antibody is a FAM150B antibody. In some embodiments, antibodies that inhibit FAM150A- and FAM150B-mediated signaling are provided. In some such embodiments, the antibody is a FAM150A/B antibody.

In some embodiments, a FAM150A antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸M to 10⁻¹³M, e.g., from 10⁻⁹M to 10⁻¹³ M) for FAM150A. In some embodiments, a FAM150B antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹ M to 10⁻¹³ M) for FAM150B.

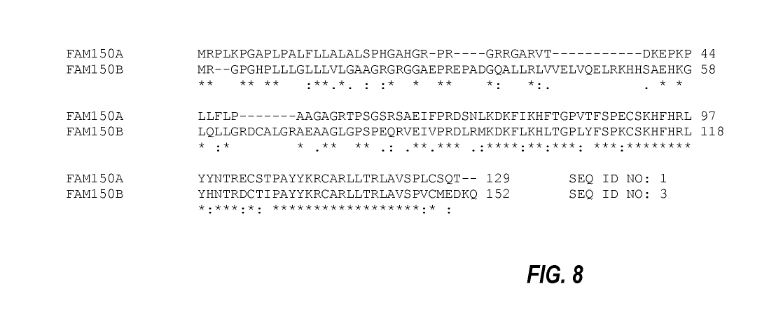

In some embodiments, a FAM150A/B antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸M to 10⁻¹³M, e.g., from 10⁻⁹M to 10⁻¹³ M) for FAM150A and a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸ M to 10⁻¹³ M, e.g., from 10⁻⁹M to 10⁻¹³ M) for FAM150B. In some embodiments, a FAM150A/B antibody has greater affinity for FAM150A than FAM150B, or has greater affinity for FAM150B than for FAM150A. In some embodiments, a FAM150A/B antibody binds to an epitope of FAM150A and an epitope of FAM150B that are conserved between the two proteins. An alignment of FAM150A and FAM150B is shown in FIG. 8. As shown in that figure, the C-terminal portions of the two ligands have a higher degree of similarity than the N-terminal portions. In some embodiments, a FAM150A/B antibody binds an epitope of FAM150A within the region of amino acids 73 to 126 of SEQ ID NO: 1. In some embodiments, a FAM150A/B antibody binds an epitope of FAM150B within a region of amino acids 94 to 147 of SEQ ID NO: 3. In some embodiments, a FAM150A/B antibody binds an epitope of FAM150A within the region of amino acids 86 to 126 of SEQ ID NO: 1. In some embodiments, a FAM150A/B antibody binds an epitope of FAM150B within a region of amino acids 107 to 147 of SEQ ID NO: 3. In some embodiments, a FAM150A/B antibody binds an epitope of FAM150A within the region of amino acids 107 to 126 of SEQ ID NO: 1. In some embodiments, a FAM150A/B antibody binds an epitope of FAM150B within a region of amino acids 128 to 147 of SEQ ID NO: 3.

In some embodiments, an antibody binds to FAM150A and/or FAM150B from multiple species. For example, in some embodiments, an antibody binds to human FAM150A and/or human FAM150B, and also binds to FAM150A and/or FAM150B from at least one mammal selected from mouse, rat, dog, guinea pig, and monkey.

In some embodiments, the antibody is an LTK antibody. In some embodiments, an LTK antibody binds to LTK extracellular domain (ECD). In some embodiments, an LTK antibody inhibits binding of FAM150A and/or FAM150B to LTK. In some embodiments, an LTK antibody inhibits FAM150A- and/or FAM150B-mediated signaling. In some embodiments, an LTK antibody is an LTK agonist antibody that stimulates LTK phosphorylation. In some embodiments, an LTK antibody stimulates LTK phosphorylation in the absence of FAM150A and/or FAM150B. In some embodiments, an LTK antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M) for LTK.

In some embodiments, an antibody binds to LTK from multiple species. For example, in some embodiments, an antibody binds to human LTK, and also binds to LTK from at least one mammal selected from mouse, rat, dog, guinea pig, and monkey.

In some embodiments, the antibody is an ALK antibody. In some embodiments, an ALK antibody binds to ALK extracellular domain (ECD). In some embodiments, an ALK antibody inhibits binding of FAM150A and/or FAM150B to ALK. In some embodiments, an ALK antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M) for ALK.

Humanized Antibodies

In some embodiments, a FAM150 antibody (such as a FAM150A antibody, a FAM150B antibody, or a FAM150A/B antibody), an LTK antibody, or an ALK antibody is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

As noted above, a humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also comprises a human heavy chain constant region and/or a human light chain constant region.

Chimeric Antibodies

In some embodiments, a FAM150 antibody (such as a FAM150A antibody, a FAM150B antibody, or a FAM150A/B antibody), an LTK antibody, or an ALK antibody is a chimeric antibody. In some embodiments, a FAM150 antibody, an LTK antibody, or an ALK antibody comprises at least one non-human variable region and at least one human constant region. In some such embodiments, all of the variable regions of a FAM150 antibody, an LTK antibody, or an ALK antibody are non-human variable regions, and all of the constant regions of the FAM150 antibody, LTK antibody, or ALK antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

Human Antibodies

In some embodiments, a FAM150 antibody (such as a FAM150A antibody, a FAM150B antibody, or a FAM150A/B antibody), an LTK antibody, or an ALK antibody is a human antibody. Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

Human Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. See, e.g., SEQ ID NO: 17. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1): 105-108 (1993). In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

### Exemplary Properties of Antibodies

Exemplary Properties of FAM150 Antibodies

In some embodiments, a FAM150A antibody binds to FAM150A and inhibits FAM150A-mediated signaling. In some embodiments, a FAM150A antibody blocks binding of FAM150A to LTK. In some embodiments, a FAM150A antibody binds to FAM150A with a binding affinity (K_{D}) of less than 50 nM, less than 20 nM, less than 10 nM, or less than 1 nM.

In some embodiments, a FAM150B antibody binds to FAM150B and inhibits FAM150B-mediated signaling. In some embodiments, a FAM150B antibody blocks binding of FAM150B to LTK. In some embodiments, a FAM150B antibody binds to FAM150B with a binding affinity (K_{D}) of less than 50 nM, less than 20 nM, less than 10 nM, or less than 1 nM.

In some embodiments, a FAM150A/B antibody binds to FAM150A and inhibits FAM150A-mediated signaling and binds to FAM150B and inhibits FAM150B-mediated signaling. In some embodiments, a FAM150A/B antibody blocks binding of FAM150A to LTK and blocks binding of FAM150B to LTK. In some embodiments, a FAM150A/B antibody binds to FAM150A with a binding affinity (K_{D}) of less than 50 nM, less than 20 nM, less than 10 nM, or less than 1 nM. In some embodiments, a FAM150A/B antibody binds to FAM150B with a binding affinity (K_{D}) of less than 50 nM, less than 20 nM, less than 10 nM, or less than 1 nM.

Exemplary Properties of LTK Antibodies

In some embodiments, an LTK antibody binds to LTK, and inhibits FAM150A- and/or FAM150B-mediated signaling. In some embodiments, an LTK antibody binds to LTK and inhibits FAM150A- and/or FAM150B-mediated LTK phosphorylation. In some embodiments, an LTK antibody blocks binding of FAM150A and/or FAM150B to LTK. In some embodiments, an LTK antibody is an LTK agonist antibody that stimulates LTK phosphorylation. In some embodiments, an LTK antibody stimulates LTK phosphorylation in the absence of FAM150A and/or FAM150B. In some embodiments, an LTK antibody binds to LTK with a binding affinity (K_{D}) of less than 50 nM, less than 20 nM, less than 10 nM, or less than 1 nM.

Exemplary Properties of ALK Antibodies

In some embodiments, the antibody is an ALK antibody. In some embodiments, an ALK antibody binds to ALK extracellular domain (ECD). In some embodiments, an ALK antibody inhibits binding of FAM150A and/or FAM150B to ALK. In some embodiments, an ALK antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M) for ALK.

Antibody Conjugates

In some embodiments, a FAM150 antibody, LTK antibody, or ALK antibody is conjugated to a label. As used herein, a label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

In some embodiments, a label is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label is a polypeptide, the label can be expressed from the same expression vector with at least one antibody chain to produce a polypeptide comprising the label fused to an antibody chain.

### FAM150A Agents

The term “FAM150A” includes full-length FAM150A, and FAM150A fragments and FAM150A variants that are able to bind LTK and/or ALK. Similarly, FAM150A fusion molecules may comprise full-length FAM150A, FAM150A fragments, or FAM150A variants that are able to bind LTK and/or ALK. In some embodiments, a FAM150A agent stimulates LTK-mediated signaling. A FAM150A agent may include or lack a signal peptide. An exemplary FAM150A agent is human FAM150A having an amino acid sequence selected from SEQ ID NOs: 1 (with signal peptide) and 2 (without signal peptide).

FAM150A fragments include fragments comprising deletions at the N- and/or C-terminus of the full-length FAM150A, wherein the FAM150A fragment retains the ability to bind LTK. FAM150A fragments may include or lack a signal peptide.

FAM150A variants include variants comprising one or more amino acid additions, deletions, and/or substitutions, and that remain capable of binding LTK. In some embodiments, a FAM150A variant sequence is at least 80%, 85%, 90%, 92%, 95%, 97%, 98%, or 99% identical to the corresponding sequence of the parent FAM150A.

### FAM150B Agents

The term “FAM150B” includes full-length FAM150B, and FAM150B fragments and FAM150B variants that are able to bind LTK and/or ALK. Similarly, FAM150B fusion molecules may comprise full-length FAM150B, FAM150B fragments, or FAM150B variants that are able to bind LTK and/or ALK. In some embodiments, a FAM150B agent stimulates LTK-mediated signaling. FAM150B agents may include or lack a signal peptide. An exemplary FAM150B is human FAM150B having an amino acid sequence selected from SEQ ID NOs: 3 (with signal peptide) and 4 (without signal peptide).

FAM150B fragments include fragments comprising deletions at the N- and/or C-terminus of the full-length FAM150B, wherein the FAM150B fragment retains the ability to bind LTK. FAM150B fragments may include or lack a signal peptide.

FAM150B variants include variants comprising one or more amino acid additions, deletions, and/or substitutions, and that remain capable of binding LTK. In some embodiments, a FAM150B variant sequence is at least 80%, 85%, 90%, 92%, 95%, 97%, 98%, or 99% identical to the corresponding sequence of the parent FAM150B.

### LTK Extracellular Domains (ECDs)

Nonlimiting exemplary LTK ECDs include full-length LTK ECDs, LTK ECD fragments, and LTK ECD variants. LTK ECDs bind to FAM150A. In some embodiments, an LTK ECD inhibits FAM150A-mediated signaling. LTK ECDs may include or lack a signal peptide. Exemplary LTK ECDs include, but are not limited to, human LTK ECDs having amino acid sequences selected from SEQ ID NOs: 13 (with signal peptide) and 14 (without signal peptide). Nonlimiting exemplary LTK ECDs are described, e.g., in Toyoshima et al., 1993, Proc. Natl. Acad. Sci. USA, 90: 5404-5408; and Haase et al., 1991, Oncogene, 6: 2319-2325; and references cited therein.

LTK ECD fragments include fragments comprising deletions at the N- and/or C-terminus of the full-length LTK ECD, wherein the LTK ECD fragment retains the ability to bind FAM150A and/or FAM150B. LTK ECD fragments may include or lack a signal peptide.

LTK ECD variants include variants comprising one or more amino acid additions, deletions, and/or substitutions, and that remain capable of binding FAM150A and/or FAM150B. In some embodiments, an LTK ECD variant sequence is at least 80%, 85%, 90%, 92%, 95%, 97%, 98%, or 99% identical to the corresponding sequence of the parent LTK ECD.

Fusion Partners and Conjugates

In some embodiments, a FAM150A, a FAM150B, and/or an LTK ECD of the present invention may be combined with a fusion partner polypeptide, resulting in a fusion protein. These fusion partner polypeptides may facilitate purification, and may show an increased half-life in vivo. Fusion partner polypeptides that have a disulfide-linked dimeric structure due to the IgG portion may also be more efficient in binding and neutralizing other molecules than the monomeric FAM150A, FAM150B, or LTK ECD fusion protein or the FAM150A, FAM150B, or LTK ECD alone. Other suitable fusion partners for FAM150A, FAM150B, and/or LTK ECD include, for example, polymers, such as water soluble polymers, the constant domain of immunoglobulins; all or part of human serum albumin (HSA); fetuin A; fetuin B; a leucine zipper domain; a tetranectin trimerization domain; mannose binding protein (also known as mannose binding lectin), for example, mannose binding protein 1; and an Fc region, as described herein and further described in U.S. Pat. No. 6,686,179.

A fusion molecule may be prepared by attaching polyaminoacids or branch point amino acids to the FAM150A, FAM150B, or LTK ECD. For example, the polyaminoacid may be a carrier protein that serves to increase the circulation half-life of the FAM150A, FAM150B, or LTK ECD (in addition to the advantages achieved via a fusion molecule). For the therapeutic purpose of the present invention, such polyaminoacids should ideally be those that do not create neutralizing antigenic response, or other adverse responses. Such polyaminoacids may be chosen from serum album (such as HSA), an additional antibody or portion thereof, for example the Fc region, fetuin A, fetuin B, leucine zipper nuclear factor erythroid derivative-2 (NFE2), neuroretinal leucine zipper, tetranectin, or other polyaminoacids, for example, lysines. As described herein, the location of attachment of the polyaminoacid may be at the N-terminus or C-terminus, or other places in between, and also may be connected by a chemical linker moiety to the selected molecule.

Polymers

Polymers, for example, water soluble polymers, may be useful in the present invention to reduce precipitation of the FAM150A, FAM150B, or LTK ECD to which the polymer is attached in an aqueous environment, such as typically found in a physiological environment. Polymers employed in the invention will be pharmaceutically acceptable for the preparation of a therapeutic product or composition.

Suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly (β-amino acids) (either homopolymers or random copolymers), poly(n-vinyl pyrrolidone) polyethylene glycol, polypropylene glycol homopolymers (PPG) and other polyalkylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll, or dextran and mixtures thereof.

Polymers used herein, for example water soluble polymers, may be of any molecular weight and may be branched or unbranched. In some embodiments, the polymers have an average molecular weight of between 2 kDa and 100 kDa, between 5 kDa and 50 kDa, or between 12 kDa and 25 kDa. Generally, the higher the molecular weight or the more branches, the higher the polymer:protein ratio. Other sizes may also be used, depending on the desired therapeutic profile; for example, the duration of sustained release; the effects, if any, on biological activity; the ease in handling; the degree or lack of antigenicity; and other known effects of a polymer on an FAM150A, FAM150B, or LTK ECD of the invention.

In some embodiments, the present invention contemplates the chemically derivatized FAM150A, FAM150B, or LTK ECD to include mono- or poly- (e.g., 2-4) PEG moieties. Pegylation may be carried out by any of the pegylation reactions available. There are a number of PEG attachment methods available to those skilled in the art. See, for example, EP 0 401 384; Malik et al., Exp. Hematol., 20:1028-1035 (1992); Francis, Focus on Growth Factors, 3(2):4-10 (1992); EP 0 154 316; EP 0 401 384; WO 92/16221; WO 95/34326; Chamow, Bioconjugate Chem., 5:133-140 (1994); U.S. Pat. No. 5,252,714; and the other publications cited herein that relate to pegylation.

Markers

FAM150A, FAM150B, or LTK ECD of the present invention may be fused to marker sequences, such as a peptide that facilitates purification of the fused polypeptide. The marker amino acid sequence may be a hexa-histidine peptide such as the tag provided in a pQE vector (Qiagen, Mississauga, Ontario, Canada), among others, many of which are commercially available. As described in Gentz et al., Proc. Natl. Acad. Sci. 86:821-824 (1989), for instance, hexa-histidine provides for convenient purification of the fusion protein. Another peptide tag useful for purification, the hemagglutinin (HA) tag, corresponds to an epitope derived from the influenza HA protein. (Wilson et al., Cell 37:767 (1984)). Any of these above fusions may be engineered using the FAM150A, FAM150B, or LTK ECD of the present invention.

Oligomerization Domain Fusion Partners

In various embodiments, oligomerization offers some functional advantages to a fusion protein, including, but not limited to, multivalency, increased binding strength, and the combined function of different domains. Accordingly, in some embodiments, a fusion partner comprises an oligomerization domain, for example, a dimerization domain. Exemplary oligomerization domains include, but are not limited to, coiled-coil domains, including alpha-helical coiled-coil domains; collagen domains; collagen-like domains; and certain immunoglobulin domains. Exemplary coiled-coil polypeptide fusion partners include, but are not limited to, the tetranectin coiled-coil domain; the coiled-coil domain of cartilage oligomeric matrix protein; angiopoietin coiled-coil domains; and leucine zipper domains. Exemplary collagen or collagen-like oligomerization domains include, but are not limited to, those found in collagens, mannose binding lectin, lung surfactant proteins A and D, adiponectin, ficolin, conglutinin, macrophage scavenger receptor, and emilin.

Antibody Fc Immunoglobulin Domain Fusion Partners

Many Fc domains that may be used as fusion partners are known in the art. In some embodiments, a fusion partner is an Fc immunoglobulin domain. An Fc fusion partner may be a wild-type Fc found in a naturally occurring antibody, a variant thereof, or a fragment thereof. Nonlimiting exemplary Fc fusion partners include Fcs comprising a hinge and the CH2 and CH3 constant domains of a human IgG, for example, human IgG1, IgG2, IgG3, or IgG4. In some embodiments, an Fc fusion partner comprises a C237S mutation, for example, in an IgG1 constant region. See, e.g., SEQ ID NO: 17. In some embodiments, an Fc fusion partner is a human IgG4 constant region. In some such embodiments, the human IgG4 constant region comprises an S241P mutation. See, e.g., Angal et al. Mol. Immunol. 30(1): 105-108 (1993). In some embodiments, an Fc fusion partner comprises a hinge, CH2, and CH3 domains of human IgG2 with a P331S mutation, as described in U.S. Pat. No. 6,900,292. Additional exemplary Fc fusion partners also include, but are not limited to, human IgA and IgM. Certain exemplary Fc domain fusion partners are shown in SEQ ID NOs: 17 to 19.

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

Albumin Fusion Partners and Albumin-Binding Molecule Fusion Partners

In some embodiments, a fusion partner is an albumin. Exemplary albumins include, but are not limited to, human serum album (HSA) and fragments of HSA that are capable of increasing the serum half-life or bioavailability of the polypeptide to which they are fused. In some embodiments, a fusion partner is an albumin-binding molecule, such as, for example, a peptide that binds albumin or a molecule that conjugates with a lipid or other molecule that binds albumin. In some embodiments, a fusion molecule comprising HSA is prepared as described, e.g., in U.S. Pat. No. 6,686,179.

Exemplary Attachment of Fusion Partners

The fusion partner may be attached, either covalently or non-covalently, to the N-terminus or the C-terminus of the FAM150A, FAM150B, or LTK ECD. The attachment may also occur at a location within the FAM150A, FAM150B, or LTK ECD other than the N-terminus or the C-terminus, for example, through an amino acid side chain (such as, for example, the side chain of cysteine, lysine, serine, or threonine).

In either covalent or non-covalent attachment embodiments, a linker may be included between the fusion partner and the FAM150A, FAM150B, or LTK ECD. Such linkers may be comprised of at least one amino acid or chemical moiety. Exemplary methods of covalently attaching a fusion partner to an FAM150A, FAM150B, or LTK ECD include, but are not limited to, translation of the fusion partner and the FAM150A, FAM150B, or LTK ECD as a single amino acid sequence and chemical attachment of the fusion partner to the FAM150A, FAM150B, or LTK ECD. When the fusion partner and an FAM150A, FAM150B, or LTK ECD are translated as single amino acid sequence, additional amino acids may be included between the fusion partner and the FAM150A, FAM150B, or LTK ECD as a linker. In some embodiments, the linker is selected based on the polynucleotide sequence that encodes it, to facilitate cloning the fusion partner and/or FAM150A, FAM150B, or LTK ECD into a single expression construct (for example, a polynucleotide containing a particular restriction site may be placed between the polynucleotide encoding the fusion partner and the polynucleotide encoding the FAM150A, FAM150B, or LTK ECD, wherein the polynucleotide containing the restriction site encodes a short amino acid linker sequence). When the fusion partner and the FAM150A, FAM150B, or LTK ECD are covalently coupled by chemical means, linkers of various sizes may typically be included during the coupling reaction.

Exemplary methods of non-covalently attaching a fusion partner to an FAM150A, FAM150B, or LTK ECD include, but are not limited to, attachment through a binding pair. Exemplary binding pairs include, but are not limited to, biotin and avidin or streptavidin, an antibody and its antigen, etc.

Exemplary Properties of LTK ECDs and LTK ECD Fusion Molecules

In some embodiments, an LTK ECD or an LTK ECD fusion molecule binds to FAM150A, and inhibits FAM150A-mediated signaling. In some embodiments, an LTK ECD or an LTK ECD fusion molecule binds to FAM150B, and inhibits FAM150B-mediated signaling. In some embodiments, an LTK ECD or an LTK ECD fusion molecule binds to both FAM150A and FAM150B, and inhibits FAM150A- and FAM150B-mediated signaling. In some embodiments, an LTK ECD or an LTK ECD fusion molecule binds to FAM150A with a binding affinity (K_{D}) of less than 50 nM, less than 20 nM, less than 10 nM, less than 1 nM, or less than 0.1 nM. In some embodiments, an LTK ECD or an LTK ECD fusion molecule binds to FAM150B with a binding affinity (K_{D}) of less than 50 nM, less than 20 nM, less than 10 nM, or less than 1 nM. In some embodiments, an LTK ECD or an LTK ECD fusion molecule blocks binding of FAM150A to LTK. In some embodiments, an LTK ECD or an LTK ECD fusion molecule blocks binding of FAM150B to LTK. In some embodiments, an LTK ECD or an LTK ECD fusion molecule blocks binding of both FAM150A and FAM150B to LTK.

### Exemplary Properties of FAM150A Agents and FAM150B Agents

In some embodiments, a FAM150A agent or a FAM150B agent binds to LTK, and stimulates LTK-mediated signaling. In some embodiments, a FAM150A agent binds to LTK with a binding affinity (K_{D}) of less than 50 nM, less than 10 nM, less than 1 nM, or less than 0.1 nM. In some embodiments, a FAM150B agent binds to LTK with a binding affinity (K_{D}) of less than 50 nM, less than 20 nM, less than 10 nM, or less than 1 nM.

### Additional FAM150 Antagonists

In some embodiments, additional molecules that bind FAM150A, FAM150B, LTK and/or ALK are provided. Such molecules include, but are not limited to, non-canonical scaffolds, such as anti-calins, adnectins, ankyrin repeats, etc. See, e.g., Hosse et al., Prot. Sci. 15:14 (2006); Fiedler, M. and Skerra, A., “Non-Antibody Scaffolds,” pp. 467-499 in Handbook of Therapeutic Antibodies, Dubel, S., ed., Wiley-VCH, Weinheim, Germany, 2007.

### Signal Peptides

In order for some secreted proteins to express and secrete in large quantities, a signal peptide from a heterologous protein may be desirable. Employing heterologous signal peptides may be advantageous in that a resulting mature polypeptide may remain unaltered as the signal peptide is removed in the ER during the secretion process. The addition of a heterologous signal peptide may be required to express and secrete some proteins.

Nonlimiting exemplary signal peptide sequences are described, e.g., in the online Signal Peptide Database maintained by the Department of Biochemistry, National University of Singapore. See Choo et al., BMC Bioinformatics, 6: 249 (2005); and PCT Publication No. WO 2006/081430.

### Co-Translational and Post-Translational Modifications

In some embodiments, a polypeptide such as a FAM150A agent, a FAM150B agent, a FAM150 antibody (such as a FAM150A antibody, a FAM150B antibody, or a FAM150A/B antibody), an LTK antibody, an LTK ECD, an LTK ECD fusion molecule, or an ALK antibody, is differentially modified during or after translation, for example by glycosylation, sialylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH_{4;} acetylation; formylation; oxidation; reduction; and/or metabolic synthesis in the presence of tunicamycin.

Additional post-translational modifications encompassed by the invention include, for example, N-linked or O-linked carbohydrate chains; processing of N-terminal or C-terminal ends; attachment of chemical moieties to the amino acid backbone; chemical modifications of N-linked or O-linked carbohydrate chains; and addition or deletion of an N-terminal methionine residue as a result of prokaryotic host cell expression.

### Nucleic Acid Molecules Encoding LTK Agonists or FAM150 Antagonists

Nucleic acid molecules are provided, wherein the nucleic acid molecules comprise polynucleotides that encode one or more chains of an antibody described herein, such as a FAM150 antibody, an LTK antibody, or an ALK antibody. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

In some embodiments, nucleic acid molecules comprising polynucleotides that encode a FAM150A agent of a FAM150B agent. Nucleic acid molecules comprising polynucleotides that encode fusion molecules in which the FAM150A or FAM150B and the fusion partner are translated as a single polypeptide are also provided.

In some embodiments, a polynucleotide encoding a FAM150A agent or a FAM150B agent comprises a nucleotide sequence that encodes a signal peptide, which, when translated, will be fused to the N-terminus of the polypeptide. As discussed above, the signal peptide may be the native signal peptide, or may be another heterologous signal peptide. In some embodiments, the nucleic acid molecule comprising the polynucleotide encoding the gene of interest is an expression vector that is suitable for expression in a selected host cell.

In some embodiments, nucleic acid molecules comprising polynucleotides that encode LTK ECDs or LTK ECD fusion molecules are provided. Nucleic acid molecules comprising polynucleotides that encode LTK ECD fusion molecules in which the LTK ECD and the fusion partner are translated as a single polypeptide are also provided.

In some embodiments, a polynucleotide encoding an LTK ECD comprises a nucleotide sequence that encodes a signal peptide, which, when translated, will be fused to the N-terminus of the LTK ECD. As discussed above, the signal peptide may be the native LTK signal peptide, or may be another heterologous signal peptide. In some embodiments, the nucleic acid molecule comprising the polynucleotide encoding the gene of interest is an expression vector that is suitable for expression in a selected host cell.

Nucleic acid molecules may be constructed using recombinant DNA techniques conventional in the art. In some embodiments, a nucleic acid molecule is an expression vector that is suitable for expression in a selected host cell.

### Polypeptide Expression and Production

Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

Vectors comprising polynucleotides that encode a FAM150A agent and/or a FAM150B agent are provided. Vectors comprising polynucleotides that encode fusion molecules comprising FAM150A or FAM150B are also provided. Such vectors include, but are not limited to, DNA vectors, phage vectors, viral vectors, retroviral vectors, etc.

Vectors comprising polynucleotides that encode LTK ECDs are provided. Vectors comprising polynucleotides that encode LTK ECD fusion molecules are also provided. Such vectors include, but are not limited to, DNA vectors, phage vectors, viral vectors, retroviral vectors, etc.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

In some embodiments, a vector is chosen for in vivo expression of a FAM150A antagonist in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Similarly, in various embodiments, FAM150A, FAM150B, LTK ECDs and/or fusion molecules comprising any of those may be expressed in prokaryotic cells, such as bacterial cells; or in eukaryotic cells, such as fungal cells, plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains, light chains, FAM150A, FAM150B, LTK ECDs, and/or fusion molecules. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Purification of Polypeptides

The antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

FAM150A, FAM150B, LTK ECDs, and fusion molecules comprising any of those may be purified by any suitable method. Such methods include, but are not limited to, the use of affinity matrices or hydrophobic interaction chromatography. Suitable affinity ligands include any ligands that bind to LTK (such as FAM150A or FAM150B), polypeptides that bind to FAM150A or FAM150B (such as an LTK ECD or LTK ECD fusion molecule) or that bind to the fusion partner, or antibodies thereto. Further, a Protein A, Protein G, Protein A/G, or an antibody affinity column may be used to bind to an Fc fusion partner to purify a fusion molecule.

In some embodiments, hydrophobic interactive chromatography, for example, a butyl or phenyl column, is also used for purifying some polypeptides. Many methods of purifying polypeptides are known in the art.

Cell-Free Production of Polypeptides

In some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Methods of Identifying FAM150 Antagonists

In some embodiments, methods of identifying FAM150 antagonists are provided. In some embodiments, a method comprises contacting a candidate molecule (i.e., a molecule being tested for antagonist activity) with LTK, an LTK ECD, or an LTK ECD fusion molecule (collectively referred to as an “LTK molecule”). In some embodiments, a method further comprises contacting the candidate molecule/LTK molecule mixture with a FAM150A agent and/or a FAM150B agent (collectively referred to as a “FAM150 molecule”). In some embodiments, a method comprises contacting the candidate molecule with the FAM150 molecule, and then contacting the candidate molecule/FAM150 molecule mixture with an LTK molecule. In some embodiments, a method comprises contacting a candidate molecule with an LTK molecule and a FAM150 molecule approximately simultaneously. In some embodiments, a method comprises forming a first composition comprising an LTK molecule and FAM150 molecule, and then contacting the candidate molecule with the first composition. One skilled in the art will recognize that the order in which the components are contacted with one another may be varied according to the assay design.

In some embodiments, the LTK molecule is a full length LTK, for example, an LTK expressed on the surface of a cell. In some embodiments, the LTK molecule is a soluble LTK, such as an LTK ECD or LTK ECD fusion molecule. In some embodiments, the FAM150 molecule is FAM150A or FAM150B. In some embodiments, the FAM150 molecule is a FAM150A fusion molecule or a FAM150B fusion molecule. In some embodiments, an assay comprises contacting the LTK molecule and/or the candidate molecule with both a FAM150A agent and a FAM150B agent. In some such embodiments, the assay is designed to identify FAM150A/B antagonists (e.g., antagonists that block binding of both FAM150A and FAM150B to LTK and/or inhibits both FAM150A- and FAM150B-induced LTK phosphorylation, etc.).

In some embodiments, after the candidate molecule has been contacted with the LTK molecule and/or the FAM150 molecule, an assay or assays are carried out to detect FAM150 molecule binding to the LTK molecule and/or LTK phosphorylation. Nonlimiting exemplary assays for detecting FAM150 molecule binding to an LTK molecule include ELISA assays, surface plasmon resonance assays (e.g., Biacore), flow cytometry-based assays (for example, when one or more components are bound to beads, or LTK is expressed on the surface of a cell), etc. Many methods of detecting protein-protein binding are known in the art, and one skilled in the art can select a suitable assay method. Further, various reagents may be used for detection as needed, including antibodies (with or without labels), secondary antibodies (with or without labels), labeled assay components (including, but not limited to, labeled FAM150 molecule and/or labeled LTK molecule), etc.

Nonlimiting exemplary assays for detecting LTK phosphorylation include immunoassays using phosphorylation-specific antibodies (such as in Western blot of ELISA format), assays involving radiolabeled ATP and detection of radiolabeled phosphorylated protein, and detection of downstream signaling, such as LTK phosphorylation-dependent expression of a reporter gene. Many methods of detecting protein phosphorylation are known in the art, and one skilled in the art can select a suitable assay method. Further, various reagents may be used for detecting phosphorylation as needed, including antibodies (with or without labels), secondary antibodies (with or without labels), reporter genes (including detectable reporter genes, such as β-gal and GFP, etc.), labeled reagents (such as radiolabeled ATP), etc.

In some embodiments, methods of identifying FAM150 antagonists comprise comparing the extent of LTK molecule/FAM150 molecule binding in the presence and absence of the candidate molecule. In some embodiments, when LTK molecule/FAM150 molecule binding is reduced in the presence of the candidate molecule relative to the binding in the absence of the candidate molecule, the candidate molecule is a FAM150 antagonist. In some embodiments, binding between the LTK molecule and the FAM150 molecule is reduced by at least 20%, at least 30%, at least 40%, at least 50%, at least 60%, at least 70%, at least 80%, or at least 90% in the presence of the candidate molecule. In some such embodiments, the candidate molecule is a FAM150 antagonist.

In some embodiments, methods of identifying FAM150 antagonists comprise comparing the extent of LTK phosphorylation (or a downstream effect of LTK phosphorylation, such as reporter gene expression) in the presence and absence of the candidate molecule. In some embodiments, when LTK phosphorylation (or a downstream effect of LTK phosphorylation, such as reporter gene expression) is reduced in the presence of the candidate molecule relative to the binding in the absence of the candidate molecule, the candidate molecule is a FAM150 antagonist. In some embodiments, phosphorylation of LTK (or a downstream effect of phosphorylation of LTK) is reduced by at least 20%, at least 30%, at least 40%, at least 50%, at least 60%, at least 70%, at least 80%, or at least 90% in the presence of the candidate molecule, as compared to the absence of the candidate molecule. In some such embodiments, the candidate molecule is a FAM150 antagonist.

In various embodiments, if the candidate molecule inhibits binding of FAM150A to LTK, the candidate molecule is a FAM150A antagonist, if the candidate molecule inhibits binding of FAM150B to LTK, the candidate molecule is a FAM150B antagonist, if the candidate molecule inhibits binding of FAM150A and FAM150B to LTK, the candidate molecule is a FAM150A/B antagonist, etc.

Exemplary classes of candidate molecules include, but are not limited to, antibodies, peptides, small molecules, and aptamers. In some embodiments, a candidate molecule is an antibody that is known to bind to LTK (i.e., an LTK antibody). In some embodiments, a candidate molecule is an antibody that is known to bind to FAM150A and/or FAM150B (i.e., a FAM150A antibody, a FAM150B antibody, or a FAM150A/B antibody).

In some embodiments, methods of determining whether an LTK antibody is a FAM150 antagonist are provided. In such embodiments, the LTK antibody is tested in the assays described above as the candidate molecule. In some embodiments, methods of determining whether an LTK antibody blocks binding of FAM150A and/or FAM150B are provided. Such methods comprise, in some embodiments, contacting an LTK antibody with an LTK molecule and a FAM150 molecule, and detecting binding of the LTK molecule to the FAM150 molecule in the presence of the antibody, e.g., as described above and herein.

In any of the embodiments described above, an LTK molecule may be replaced with an ALK molecule (i.e., ALK, an ALK ECD, or an ALK ECD fusion molecule). Briefly, an ALK ECD refers to an ALK polypeptide that lacks the intracellular and transmembrane domains, with or without a signal peptide. “ALK ECD” includes full-length ALK ECDs, ALK ECD fragments, and ALK ECD variants, wherein the fragments and variants retain the ability to bind FAM150A and/or FAM150B. An ALK ECD fusion molecule comprises an ALK ECD and one or more fusion partners as defined herein.

### Articles of Manufacture

In some embodiments, an article of manufacture or a kit containing materials useful for the detection of a biomarker (e.g., FAM150A, FAM150B, LTK or ALK) or for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. In some embodiments, the container holds a composition which is by itself or combined with another composition effective for treating, preventing and/or diagnosing the condition and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises an LTK agonist (such as an LTK agonist antibody, a FAM150A agent, and/or a FAM150B agent), or FAM150 antagonist of the invention; and (b) a second container with a composition contained therein, wherein the composition comprises an additional therapeutic agent. The article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the molecules of the present invention can be packaged alone or in combination with other therapeutic compounds as a kit. In one embodiment, the therapeutic compound is an anti-cancer agent. In another embodiment, the therapeutic compound is an immunosuppressive agent. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### In Vitro Screen to Identify Ligands for Leukocyte Tyrosine Kinase (LTK)

An assay was developed to identify ligands for the orphan receptor leukocyte tyrosine kinase (LTK). The assay was designed to detect phosphorylation of the receptor on tyrosine residues in the kinase domain following activation by a ligand.

Stable cell lines were generated in human HEK293T cells by transfection of the full length hLTK cDNA (codon optimized sequence) with an HA tag at the C-terminus, in vector pcDNA5/FRT (Invitrogen, Carlsbad, Calif.). Transfected cells were generated using the Flip-In plasmid system (Invitrogen, Carlsbad, Calif.) and selected for stable integration. Cells were grown and maintained at 37° C. in 5% CO₂ in media comprising Eagle's Minimum Essential Medium (EMEM; American Type Culture Collection, Manassas, Calif.), 10% fetal bovine serum (Corning Cellgro®, Mannassas, Calif.), 1% penicillin-streptomycin (Corning Cellgro®, Mannassas, Calif.)+100 μg/ml hygromycin (Invitrogen, Carlsbad, Calif.). Stably transfected cells may be referred to as “LTK-293 cells.”

Supernatants enriched in secreted proteins were generated by transfecting cDNAs into HEK293T cells in DMEM (Corning Cellgro®, Mannassas, Calif.) supplemented with 5% FBS (Corning Cellgro®, Mannassas, Calif.). Transfections were carried out using Fugene® 6 (Promega, Madison, Wis.) following the manufacturer's recommended conditions for 40 hours followed by a media change into fresh medium for 48 hours. The supernatants from transfected cells as well as supernatant from a negative control protein-expressing plasmid were collected and used to treat the LTK-293 overexpression cells.

Cells were plated at 50,000 LTK-293 cells per well in 175 μl growth medium in a BD Biocoat Poly-D-Lysine 96-well microplate (BD Biosciences, San Jose, Calif.) and incubated 24 hours at 37° C. in 5% CO₂. Serum starvation in EMEM+0.1% FBS was performed for 24 hours by removing the culture medium and replacing it with 175 μl of starvation medium. Cells were treated with the enriched protein expression supernatants by removing the starvation medium and replacing it with 100 μl of a 1:1 mix of expressed protein supernatant and fresh starvation medium. Cells were treated for 20 minutes in a 37° C. incubator. The cell lysates were generated by removing the medium completely and adding 75 μl of cold lysis buffer (10× Cell Lysis Buffer; Cell Signaling Technology, Danvers, Mass.) diluted to 1× in water plus cOmplete, Mini, EDTA-free protease inhibitor cocktail (Roche Applied Science) and PhosSTOP phosphatase inhibitor (Roche Applied Science) into each well. The lysates were frozen at −80° C. for later ELISA processing.

ELISA assays were performed by coating white Lumitrac 600 high binding 96-well half area ELISA plates (E&K Scientific, Santa Clara, Calif.)) with anti-HA tag mouse monoclonal antibody [HA.C5] (Abcam, Cambridge, Mass.) as follows. The antibody was diluted in PBS to 4.5 μg/ml and 50 μl was dispensed per well. The plates were sealed and incubated overnight at 4° C. Plates were washed 3 times with PBST (PBS with 0.05% Tween20), then blocked with 180 μl per well of PBS+1% BSA for 1 hour at room temperature. Plates were washed 3 times with PBST. For the capture step, lysates were thawed and the samples transferred to the ELISA plates and incubated for 2 hours at room temperature. Plates were washed 6 times with PBST. 50 μl per well of a 1:9000 dilution of an HRP-conjugated anti-phospho-tyrosine antibody (R&D Systems, Minneapolis, Minn.) was added and the plates were sealed and incubated for 1 hour at room temperature. The plates were washed 6 times with PBST followed by the addition of 50 μl/well of luminescent substrate (SuperSignal ELISA Pico Chemiluminscent Substrate kit, Thermo Scientific, Rockford, Ill.). The plates were incubated for 2 minutes, and then read on an EnVision instrument (PerkinElmer, Waltham, Mass.) following the manufacturer's recommended settings.

Expression supernatants from over 4000 proteins were screened in the LTK phosphorylation assay described above. Two proteins showed significant activity in the assay, FAM150A and FAM150B.

### Example 2

### Expression and Purification of FAM150A and FAM150B

Human FAM150A and FAM150B were expressed in transiently transfected HEK 293 6E cells grown in shaker flasks in FreeStyle 293 Expression Medium (Invitrogen) at 37° C. in 5% CO₂. Cell densities ranged from 0.6×10⁶ to 2×10⁶ cells/ml. Typically, 500 ml of cell culture was grown in a 2 L flask with multiple flasks being prepared for one transfection. On the day of transfection, cells were harvested by centrifugation, the media was replaced with new media, and the cells resuspended at a cell density of 1×10⁶ cells/ml with 500 ml per 2 L flask. DNA transfection complex was made by adding 500 μg DNA into 25 ml of phosphate buffered saline (PBS) in one tube, and adding 1000 μg of linear polyethylenimine, MW 2,500 (1 mg/ml sterile stock solution, pH 7.0; Polysciences Inc., Arrington, Wis.) to 25 ml of PBS in a second tube. The contents of the two tubes were mixed and allowed to incubate for 15 minutes at room temperature to form the transfection complex. The transfection complex was transferred to the HEK 293 6E cell suspension culture, which was allowed to grow for 6-7 days at 37° C. in 5% CO₂. At 24 hours post-transfection, 20% (w/v) tryptone N1 (OrganoTechnie S.A., La Courneuve, France) was added to the culture at a final concentration of 0.5% (w/v). To produce FAM150A or FAM150B, six 2 L flasks were transfected for each construct at one time to produce approximately 3 liters of culture for each.

The HEK 293 6E cultures expressing either FAM150A or FAM150B were harvested on day 6 post-transfection. For each, culture supernatant was clarified by centrifugation at 1400 rpm for 10 minutes and then 5,000×g for 10 minutes at 4° C. The supernatant was dialyzed in a 10 kD molecular weight cut-off dialysis bag against Buffer A (10 mM potassium phosphate, pH 6.8, with 30 mM sodium chloride). The dialyzed material was loaded on two 5-ml SP Sepharose HP columns (GE Healthcare Life Sciences, Pittsburgh, Pa.) connected in tandem. The bound protein was washed with 5 column volumes of Buffer A. Bound protein was eluted from the column using a 25 column volume linear gradient elution from Buffer A to Buffer B (10 mM potassium phosphate, pH 6.8, 1 M sodium chloride). Elution fractions were analyzed by SDS-PAGE and for induction of LTK phosphorylation using a phosphorylation assay substantially as described in Example 1. Fractions enriched in FAM 150A by SDS-PAGE and induction of LTK phosphorylation activity were pooled. The pooled fractions of FAM150A were about 60-80% pure (i.e., FAM150A represented 60-80% of the protein in the pooled fractions) after one purification step. Fractions enriched in FAM150B as determined by induction of LTK phosphorylation activity alone were pooled. The pooled fractions of FAM150B were less than 1% pure (i.e., FAM150B represented less than 1% of the protein in the pooled fractions).

Pooled fractions were adjusted to 1M ammonium sulfate using a 2.4 M ammonium sulfate stock solution, pH 7.5, centrifuged at 10,000×g for 10 minutes to remove any precipitate. The supernatant was then loaded onto a 1 ml Butyl Sepharose HP column (GE Healthcare Life Sciences, Pittsburgh, Pa.) equilibrated to 1.2 M ammonium sulfate, 10 mM NaPO₄, pH 7.5. Protein was eluted in a 15 column volume gradient to 0 M ammonium sulfate. For FAM150A, eluted fractions were analyzed by SDS-PAGE stained with Coommassie blue, and fractions highly enriched in FAM150A (>90% purity) were pooled and dialyzed into 2×PBS, filter sterilized, and stored in aliquots at −80° C. For the FAM150B, the column fractions were tested for by induction of LTK phosphorylation. Active fractions were pooled based on activity, dialyzed into 2×PBS, concentrated 20-fold by ultrafiltration on a 3,000 MW Amicon membrane (EMD Millipore, Billerica, Mass.), filter sterilized, and stored at 4° C.

### Example 3

### Expression and Purification of LTK-Fc, LTK(Short)-Fc, and ALK-Fc

Human LTK-Fc, human LTK(short)-Fc, and human ALK-Fc were expressed in transiently transfected CHO-3E7 cells (CHO cells that stably express EBNA1). CHO-3E7 cells were grown in shaker flasks in HyClone SFM4CHO medium with 8 mM glutamine (Thermo Scientific, Waltham, Mass.) at 37° C. in 5% CO₂. Cell densities ranged from 0.3×10⁶ to 4×10⁶ cells/ml. Typically, 500 ml of cell culture was grown in a 2 L flask with multiple flasks being prepared for one transfection. On the day of transfection the cells were harvested by centrifugation, and the cells were resuspended at a cell density of 4×10⁶ cells/ml in CD DG44 medium (Life Technologies, Carlsbad, Calif.) supplemented with 8 mM glutamine and 0.18% pluronic F-68, with 500 ml per 2 L flask. DNA transfection complex was made by adding 750 μg of DNA into 25 ml of CD DG44 medium in one tube, and adding 3750 μg of 3 mg/ml PeiMAX (polyethylenimine linear, MW 25 kDa free base form (nominally MW 40 kDa; 3 mg/ml sterile stock solution, pH 7.0; Polysciences, Inc., Arrington, Wis.) to 25 ml of CD DG44 media in a second tube. The contents of the two tubes were mixed and allowed to incubate for 15 minutes at room temperature to form the transfection complex. The transfection complex was transferred to the CHO-3E7 cell suspension culture, which was allowed to grow for 6-7 days at 37° C. in 5% CO₂. At 24 hours post-transfection, 165 ml of additional CD DG44 media supplemented with 8 mM glutamine and 0.18% pluronic F-68 was added, along with tryptone N1 (OrganoTechnie S.A., La Courneuve, France) to a final concentration of 1.0% (w/v). To produce LTK-Fc, LTK(short)-Fc, and ALK-Fc, two 2 L flasks were transfected at one time to produce approximately 1.5 liters of culture fluid for each.

The CHO 3E7 cultures expressing human LTK-Fc (SEQ ID NO: 23 following signal sequence cleavage), human LTK(short)-Fc (SEQ ID NO: 29 following signal sequence cleavage), or human ALK-Fc (SEQ ID NO: 25 following signal sequence cleavage) were harvested on day 6 post-transfection. For each, culture supernatant was clarified by centrifugation at 1400 rpm for 10 minutes and then 5,000×g for 10 minutes at 4° C. The supernatant was then loaded on a 5 ml Protein A column (GE Healthcare Life Sciences, Pittsburgh, Pa.). The column was washed with phosphate-buffered saline containing 0.5 M NaCl, and then eluted with a linear 15 column volume gradient to 0.1 M glycine, pH 2.7, 0.5 M NaCl in 1.5 ml fractions. To neutralize the low pH elution buffer, 150 μl of 1 M Tris, pH 8.0, was added to each tube prior to fraction collection. Fractions enriched in non-aggregated protein were identified by SDS-PAGE analysis stained with Coomassie blue. The fractions enriched in protein were pooled and ammonium sulfate was added to 1 M. The pooled fractions were then further purified on a Butyl Sepharose HP column (GE Healthcare Life Sciences, Pittsburgh, Pa.) equilibrated to 1.2 M ammonium sulfate, 10 mM NaPO₄, pH 7.5. Protein was eluted with a 15 column volume gradient to 10 mM NaPO₄ pH 7.5. Enriched fractions were identified by SDS-PAGE analysis and then pooled based on purity and low aggregation, dialyzed into 1×PBS, filter sterilized, aliquoted, and stored at −80° C.

### Example 4

### FAM150A and FAM150B Induce Phosphorylation of LTK

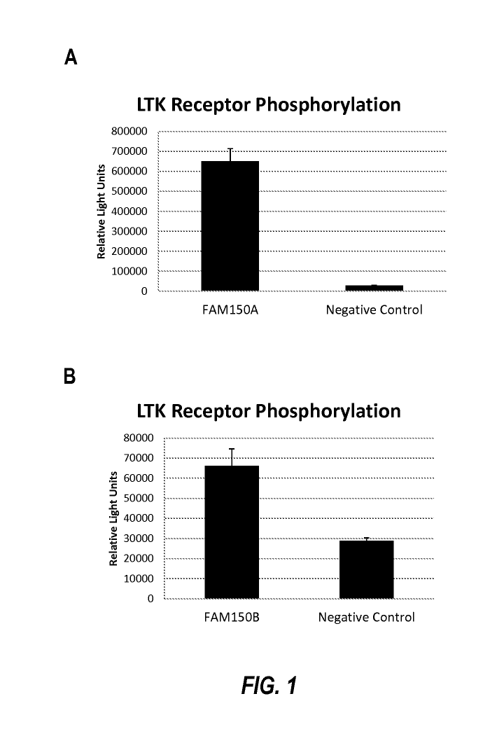

A phosphorylation assay to determine induction of LTK phosphorylation by FAM150A or FAM150B was carried out substantially as described in Example 1, using supernatant from HEK293T cells transfected with cDNA encoding FAM150A or FAM150B. FIG. 1A shows that FAM150A induced LTK phosphorylation by more than 20-fold relative to an unrelated control protein. FIG. 1B shows that FAM150B induced LTK phosphorylation by a little more than two-fold relative to an unrelated control protein. Further, in a control experiment, it was confirmed that neither FAM150A nor FAM150B induces phosphorylation of an unrelated receptor, EGFR (data not shown).

FAM150A protein purified as described in Example 2 was tested for its ability to induce phosphorylation of LTK in a phosphorylation assay substantially as described in Example 1.

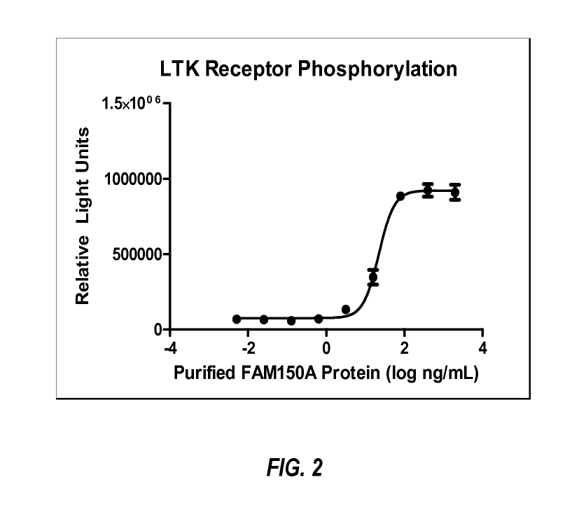

As shown in FIG. 2, FAM150A induced LTK phosphorylation in a dose-dependent manner. The EC50 for FAM150A-induced LTK phosphorylation in that experiment was 1.96 nM.

Induction of LTK phosphorylation by FAM150A was further confirmed in SK-N-SH cells (ATCC, Rockville, Md.), which endogenously express LTK. SK-N-SH cells were seeded at 5×10⁶ cells per well in 6-well culture plates in DMEM with 10% FBS and grown overnight at 37° C. The culture medium was replaced with starvation medium (DMEM, 0.1% FBS) and the cells were starved for 24 hours at 37° C. FAM150A (200 ng/ml), with or without 1 μM kinase inhibitor crizotinib (Cat No. S1068, Selleckchem, Houston, Tex.), was then added to the cells for 20 minutes. At the end of the incubation, cells were washed with cold PBS, and 250 μl of cell lysis buffer (Cat. No. 9803S, Cell Signaling Technology, Beverly, Mass.) containing protease inhibitor cocktail (Cat. No. P8340, Sigma-Aldrich, St. Louis, Mo.) and phosphatase inhibitor cocktail 2 (Cat. No. 5726, Sigma-Aldrich, St. Louis, Mo.) was added to each well.

Cell lysate was immunoprecipitated with a sheep anti-human LTK affinity purified polyclonal antibody (R&D Systems, Minneapolis, Minn.) overnight, and the immunoprecipitate was separated on a reducing SDS-PAGE gel. Tyrosine phosphorylation was detected by blotting with a mouse anti-phosphotyrosine monoclonal antibody conjugated to horse radish peroxidase (R&D Systems, Minneapolis, Minn.), and the signal was developed according to the manufacturer's instructions. Whole cell lysate was run on a separate reducing SDS-PAGE gel and probed for ERK1/2 phosphorylation using an anti-phospho-p44/42 MAPK (ERK1/2) (Thr202/Tyr204) antibody (Cell Signaling Technology, Danvers, Mass.). B-actin was detected as a loading control using an anti-β-actin antibody conjugated to horse radish peroxidase (Abcam, Cambridge, Mass.).

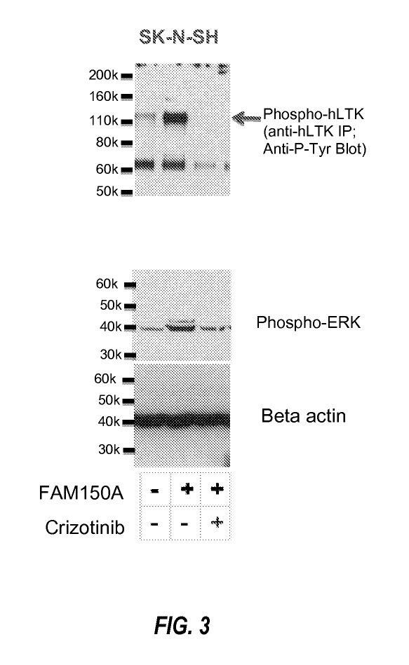

The results of that experiment are shown in FIG. 3. LTK phosphorylation increased in the presence of FAM150A. Addition of kinase inhibitor crizotinib appeared to strongly inhibit FAM150A-induced LTK phosphorylation. FIG. 3 also shows that ERK1/2 phosphorylation is stimulated in the presence of FAM150A, and that stimulation is repressed by crizotinib. ERK1/2 is a downstream indicator for many signal transduction pathways.

### Example 5

### FAM150A and FAM150B Affinity for LTK and ALK

FAM150A and FAM150B Binding to Human LTK and ALK

The kₐ, k_{d}, and K_{D} for binding of human LTK extracellular domain (ECD)-Fc (SEQ ID NO: 23) and human ALK ECD-Fc (SEQ ID NO: 25) to human FAM150A and FAM150B was determined as follows.

Binding kinetics of FAM150A and FAM150B to LTK ECD-Fc and ALK ECD Fc fusion proteins was determined using Biacore T100 surface plasmon resonance (SPR) (GE Healthcare Life Sciences, Piscataway, N.J.). LTK ECD-Fc and ALK ECD-Fc (and a control unrelated protein-Fc fusion) were captured on a CM4 sensor chip immobilized with anti-human IgG antibody using the Human Antibody Capture Kit (GE Healthcare Life Sciences, Piscataway, N.J.). 10 mM HEPES buffered saline, pH 7.4, 150 mM NaCl, 0.005% Tween-20 (HBS-P, GE Healthcare Life Sciences, Piscataway, N.J.) was used as the running and dilution buffer. Capture levels of the ECD-Fc fusions were adjusted to approximately 300-500RU so that binding values would be greater than the levels of non-specific binding to the reference flow cell. FAM150A and FAM150B were injected at eight concentrations (300 nM, 100 nM, 33.3 nM, 11.1 nM, 3.7 nM, 1. 2 nM, 0.41 nM, 0.13 nM and 0 nM) for 90 seconds or 180 seconds. The short and long association method was used to increase the accuracy of the binding kinetics.

The association constant, dissociation constant, and affinity for FAM150A for LTK ECD-Fc and ALK ECD-Fc fusions was calculated using the Biacore T100 Evaluation software package using standard double referencing technique and the 1:1 binding model or steady state affinity model. The affinities of FAM150B for LTK ECD-Fc and ALK ECD-Fc could not be calculated in this assay due to high non-specific binding. No specific binding of FAM150A or FAM150B were observed for the unrelated protein-Fc fusion. The results of the experiment are shown in Table 1.

TABLE 1
FAM150A binding affinity for human
LTK ECD-Fc and ALK ECD-Fc fusions

| | | | | K_{D} |
|---|---|---|---|---|
| Receptor | Ligand | kₐ (1/Ms) | k_{d} (1/s) | (nM) |
| ALK ECD-Fc | FAM150A | | | 280 |
| LTK ECD-Fc | FAM150A | >1 × 10⁷ | 3.98 × 10⁻⁰⁴ | <0.04 |

Although there was high non-specific binding of FAM150B to the Biacore chip, there was specific binding to ALK ECD-Fc and LTK ECD-Fc fusions when compared to the reference flow cell and a flow cell containing an unrelated ECD-Fc fusion protein. All ECD-Fc fusions are captured at similar densities. Specific binding is apparent in the association phase in the sensorgrams by the greater increase in binding signal in addition to the refractive index change over the period of injecting FAM150B over the flow cell whereas the reference and control flow cells have increases that are due only to the refractive index change and non-specific binding. A report point before the end of the injection, but during the injection, is commonly referred as the “binding” report point. The binding report points for ALK ECD-Fc and LTK ECD-Fc fusions have higher values than the reference. Residual binding after the injection of FAM150B is complete, referred to as the “binding stability” report point, is also higher for both ALK ECD-Fc and LTK ECD-Fc fusion proteins than the reference and control flow cells. The results are shown in Tables 2A and B.

TABLES 2A and B
FAM150B binding is specific to ALK ECD-Fc and LTK ECD-Fc

| | Reference | | ALK ECD-Fc | | LTK ECD-Fc | | Unrelated ECD-Fc | |
|---|---|---|---|---|---|---|---|---|
| | | blank | | blank | | blank | | blank |
| nM | binding | subtracted | binding | subtracted | binding | subtracted | binding | subtracted |
| FAM150B | (RU) | (RU) | (RU) | (RU) | (RU) | (RU) | (RU) | (RU) |

A. Binding values (RU) for ECD-Fc and unrelated ECD-Fc

| | | | | | | | | |
|---|---|---|---|---|---|---|---|---|
| 0 | 181.0 | — | 183.4 | — | 185.2 | — | 184.6 | — |
| 11.1 | 213.4 | 32.4 | 231.7 | 48.3 | 228.3 | 43.1 | 212.2 | 27.7 |
| 33.3 | 276.0 | 95.0 | 298.9 | 115.5 | 288.6 | 103.4 | 271.7 | 87.1 |

B. Binding Stability values (RU) for ECD-Fc and reference

| | | | | | | | | |
|---|---|---|---|---|---|---|---|---|
| 0 | 1.45 | — | 5.5 | — | 5.775 | — | 5.05 | — |
| 11.1 | 4.75 | 3.3 | 9.65 | 4.2 | 8.8 | 3.0 | 7.05 | 2.0 |
| 33.3 | 5.4 | 4.0 | 12.9 | 7.4 | 10.6 | 4.8 | 6.4 | 1.4 |

FAM150A Binding to Human and Mouse LTK

The kₐ, k_{d}, and K_{D} for binding of human FAM150A to human LTK ECD-Fc (SEQ ID NO: 23) and mouse LTK ECD-Fc (SEQ ID NO: 25) was determined as follows. Binding kinetics of FAM150A to human and mouse LTK ECD-Fc fusion proteins was determined using Biacore T100 SPR (GE Healthcare Life Sciences, Piscataway, N.J.). LTK ECD-Fc fusions were captured on a CM4 sensor chip immobilized with Protein A (Thermo Scientific, Rockford, Ill.). HBS-P (GE Healthcare Life Sciences, Piscataway, N.J.) was used as the running and dilution buffer. Capture levels of the ECD-Fc fusions were adjusted to approximately 300-500RU so that binding values would be greater than the levels of non-specific binding to the reference flow cell. FAM150A was injected at eight concentrations (100 nM, 33.3 nM, 11.1 nM, 3.7 nM, 1.2 nM, 0.41 nM, 0.13 nM, 0.046 nM and 0 nM) for 120 seconds. The association constant, dissociation constant, and affinity of FAM150A for LTK Fc-ECD fusions was calculated using the Biacore T100 Evaluation software package using the 1:1 binding model. The results of are shown in Table 3.

TABLE 3
FAM150A binding affinity for human and mouse LTK ECD-Fc fusions

| ECD-Fc | kₐ (1/Ms) | k_{d} (Vs) | K_{D} (nM) |
|---|---|---|---|
| Human LTK | >1 × 10⁷ | 2.78 × 10⁻⁴ | <0.028 |
| Mouse LTK | 8.45 × 10⁶ | 7.99 × 10⁻³ | 0.95 |

Human FAM150A bound to mouse LTK ECD-Fc with almost 30-fold lower affinity than to human LTK ECD-Fc.

### Example 6

### FAM150A and FAM150B Expression in Cell Lines and Tissues

FAM150A mRNA and FAM150B mRNA expression was determined in various cancer cell lines as follows. Cell lines were obtained from the Health Protection Agency (Salisbury, UK), Leibniz Institute DSMZ-German Collection of Microorganisms and Cell Cultures (Berlin, Germany), and the American Type Culture Collection (Manassas, Va.) and cultured according to the vendors' instructions. RNA was extracted from cell lines using the RNAeasy® mini kit (Qiagen, Germany). Extracted RNA was treated with DNAse I prior to creating cDNA with random hexamer priming and reverse transcriptase using the QuantiTect Reverse Transcription Kit (Qiagen, Germany). FAM150A and FAM150B mRNA expression was determined using QuantiTect Primer Assays (Qiagen, Germany) employing a human GUSB control reference QuantiTect Primer Assay (Qiagen, Germany). QuantiTect SYBR Green PCR Kits (Qiagen, Germany) were used to quantify mRNA expression levels using real-time qRT-PCR and an ABI Prism ViiA™ 7 Real-Time PCR System (Applied Biosystems, Foster City, Calif.). Relative gene expression quantification was calculated according to the comparative Ct method using human GUSB as a reference and commercial RNA controls (Stratagene, La Jolla, Calif.). Relative quantification was determined according to the formula: 2^{-(ΔCt sample-ΔCt calibrator)}.

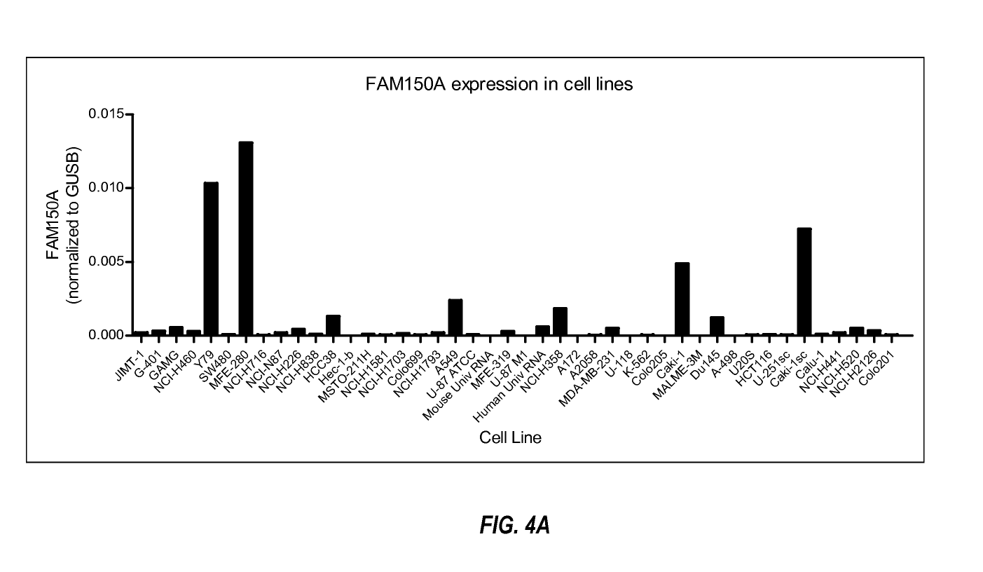

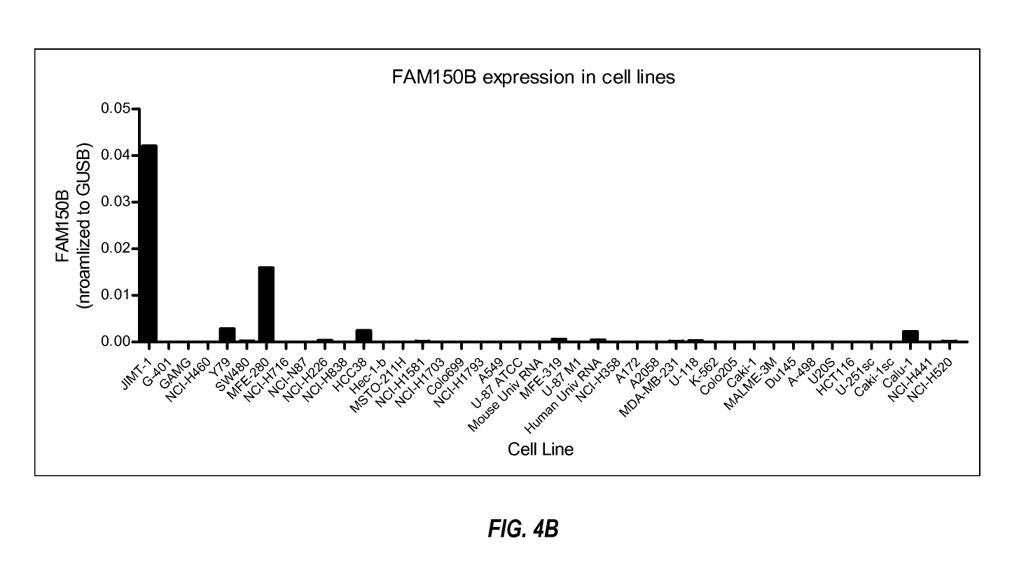

The results of that experiment are shown in FIG. 4. As shown in FIG. 4A, FAM150A showed highest expression in Y79 (human retinoblastoma cell line), MFE-280 (human endometrial carcinoma cell line), Caki-1 (human kidney clear cell carcinoma cell line), and Caki-1sc (human kidney clear cell carcinoma cell line derived from a subcutaneous Caki-1 xenograft tumor). FAM150A showed moderate expression in HCC38 (human breast ductal carcinoma cell line), A549 (human lung carcinoma cell line), and NCI-H358 (human lung bronchioalveolar carcinoma cell line). As shown in FIG. 4B, FAM150B showed highest expression in JIMT-1 (human breast carcinoma cell line) and MFE-280 (human endometrial carcinoma cell line). FAM150B was also expressed in Y79 (human retinoblastoma cell line), HCC38 (human breast ductal carcinoma cell line), and Calu-1 (lung epidermoid carcinoma cell line). Expression of FAM150A and FAM150B in cancer cells suggests that FAM150A and FAM150B may be appropriate targets for treating cancer.

FAM150A and FAM150B mRNA expression was also determined in a human immune cell cDNA panel (AllCells, Emeryville, Calif.), substantially as described above.

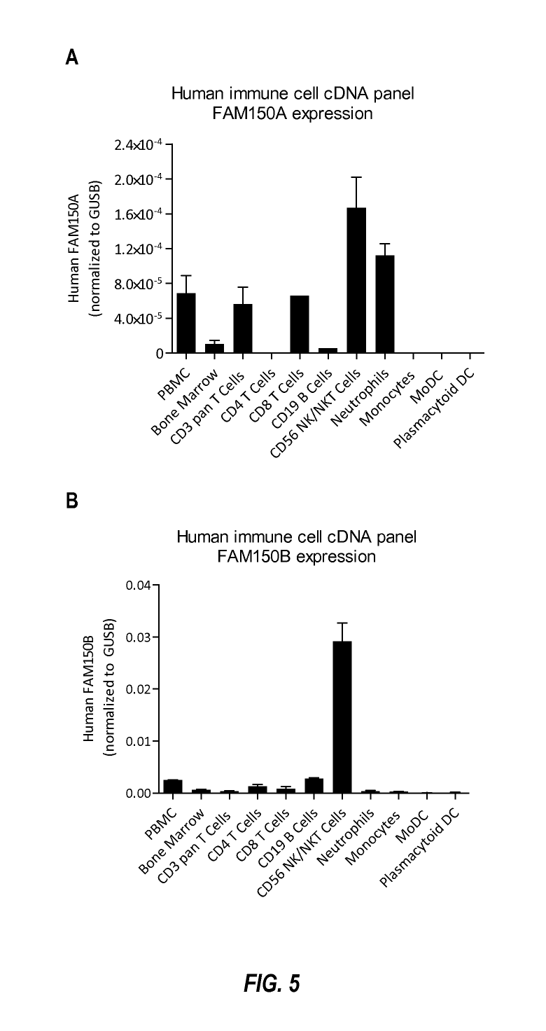

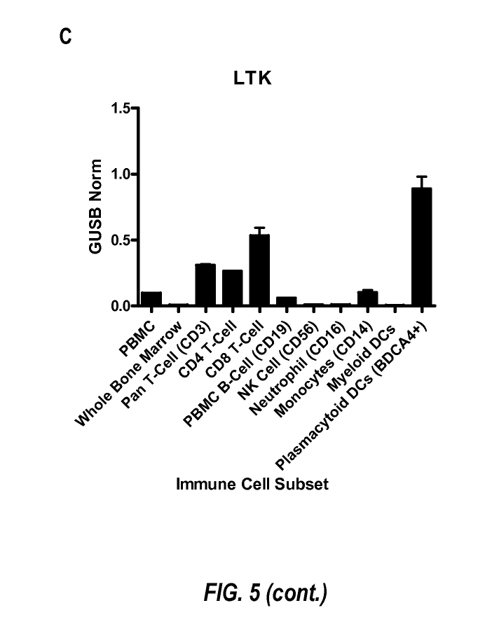

The results of that experiment are shown in FIG. 5. As shown in FIG. 5A, FAM150A showed highest expression in CD56 natural killer and natural killer T cells (CD56 NK/NKT cells) and neutrophils. FAM150A showed moderate expression in peripheral blood mononucleocytes (PBMC), CD3 pan T cells, and CD8 T cells. As shown in FIG. 5B, FAM150B showed highest expression in CD56 natural killer and natural killer T cells (CD56 NK/NKT cells). As shown in FIG. 5C, LTK showed highest expression in plasmacytoid dendritic cells (BDCA4+), and was also expressed in CD3 pan T cells, CD4 T cells, and CD8 T cells. There was detectable LTK expression in peripheral blood mononucleocytes (PBMCs), PBMC C cells (CD19), and monocytes (CD14).

Expression of LTK in BDCA+ dendritic cells, CD4 T cells, and CD8 T cells was confirmed on BioGPS (biogps.org), using dataset GeneAtlas U133A, germa (updated Nov. 19, 2012), and probe sets 217184_s_at and 207106_s_at. Expression of LTK in human immune cells suggests that LTK may be an appropriate target for treating autoimmune conditions. Previous studies have shown that LTK signaling is upstream of numerous signaling pathways involved in cell growth, survival and differentiation. Therefore, modulation of LTK signaling by addition or blockade of its ligands, FAM150A or FAM150B, has the potential for therapeutic benefit in autoimmune diseases where cells expressing LTK are known to play a pathogenic role. For example, modulation of LTK signaling in T cells may be beneficial in treating rheumatoid arthritis, psoriasis, inflammatory bowel disease, multiple sclerosis and other T cell-mediated autoimmune diseases. Modulation of LTK signaling in plasmacytoid dendritic cells may be beneficial in treating systemic lupus erythematosus, psoriasis and other plasmacytoid dendritic cell-mediated autoimmune diseases.

### Example 7

### FAM150A Induces PC12 Neurite Growth

PC12 cells were obtained from ATCC (Manassas, Va.) and cultured according to the vendor's instructions. Cells were plated at 2×10⁴ cells/cm² on 24-well polystyrene tissue culture dishes coated with type I collagen (Sigma; St. Louis, Mo.). Twenty-four hours post-plating, cells were transfected with a plasmid encoding the human LTK gene (HA-tagged LTK in vector pcDNA5/FRT) or green fluorescent protein (GFP) using Lipofectamine 2000 (Life Technologies, Carlsbad, Calif.) according to manufacturer's instructions. Twenty-four hours post-transfection, purified FAM150A was added to the media at a concentration of 1 μg/ml. Addition of mouse nerve growth factor-7S (NGF-7S; Sigma; St. Louis, Mo.) at 100 ng/ml was used as a positive control. Neurite outgrowth was assessed 7-days post-FAM150A addition using bright field and fluorescent microscopy.

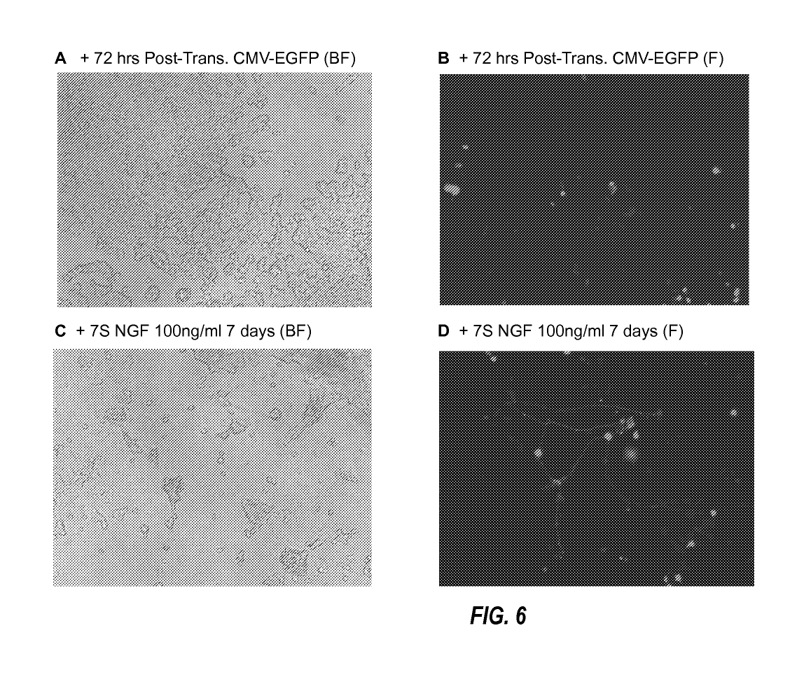

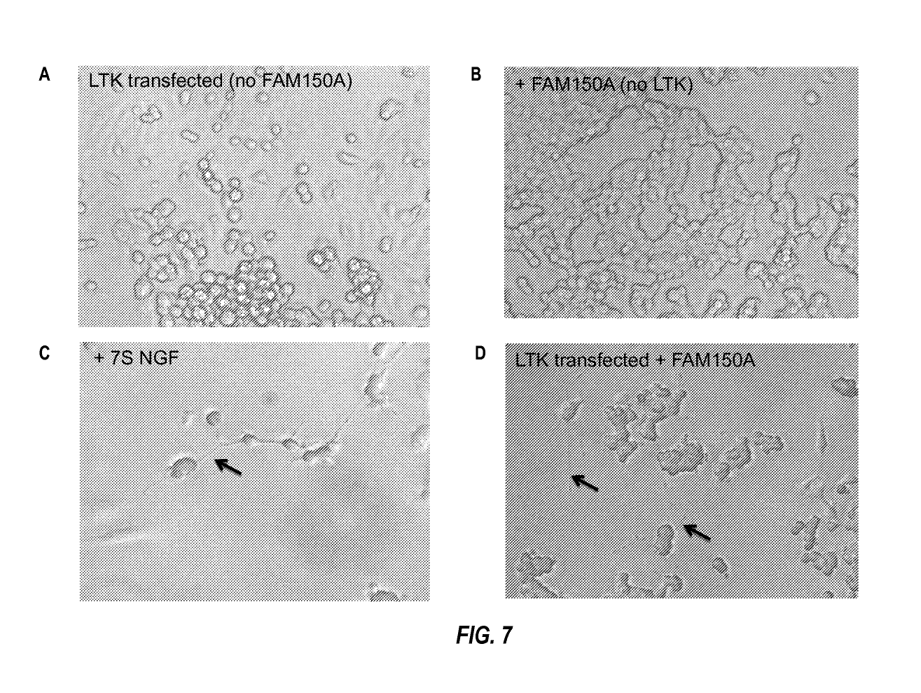

The results are shown in FIGS. 6 and 7. FIG. 6 shows neurite outgrowth in control PC12 cells transfected with EGFP and contacted with 100 ng/ml NGF-7S. FIGS. 6A and B show cells under bright field and fluorescent microscopy, respectively, without addition of NGF-7S. Neurite outgrowth is not observed. FIGS. 6C and D show cells under bright field and fluorescent microscopy, respectively, seven days after addition of NGF-7S. The cells show neurite outgrowth.

FIG. 7 shows neurite outgrowth in PC12 cells (A) transfected with LTK, (B) contacted with 1 μg/ml FAM150A without LTK transfection, (C) contacted with 100 ng/ml 7S NGF as a positive control (without LTK transfection), and (D) transfected with LTK and contacted with 1 μg/ml FAM150A. Neither LTK transfection alone nor contact with FAM150A alone induced neurite outgrowth in PC12 cells (FIGS. 7, A and B). However, transfection with LTK followed by contact with FAM150A induced neurite outgrowth (FIG. 7D), similar to the positive control, contact with 7S NGF (FIG. 7C). Examples of neurite outgrowth are indicated by arrows.

### Example 8

### FAM150A, FAM150B, and LTK Expression in Cancer

To determine in which cancers FAM150 antagonists may be particularly effective, FAM150A, FAM150B, and LTK expression was examined in TCGA (The Cancer Genome Atlas, cancergenome.nih.gov), a publicly-accessible database created as a joint effort of the National Cancer Institute and the National Human Genome Research Institute.

Elevated expression of LTK and FAM150A or FAM150B is observed in subpopulations of certain cancers. The Fisher Exact Test was used to identify statistically significant overlap between the high-expressing LTK and FAM150A subpopulations, and high-expressing LTK and FAM150B subpopulations. The calculation was done for a range of values of p, from 20% to 1%. Table 4 lists certain cancers that have an overlap in high LTK expression and high expression of FAM150A or FAM150B, with statistical significance. The overlapping high-expression subpopulation percentages (values of p) are also shown.

TABLE 4
Cancers with elevated expression of LTK and FAM150A and/or FAM150B

| Cancer | Pair | Subpopulation % |
|---|---|---|
| Breast invasive carcinoma | LTK/FAM150A | 5% |
| Ovarian serous cystadenocarcinoma | LTK/FAM150A | 5% |
| Kidney renal clear cell carcinoma | LTK/FAM150A | 3% |
| Breast invasive carcinoma | LTK/FAM150B | 20% |
| Ovarian serous cystadenocarcinoma | LTK/FAM150B | 12% |
| Colon adenocarcinoma | LTK/FAM150B | 1% |
| Bladder urothelial carcinoma | LTK/FAM150B | 13% |
| Lung squamous cell carcinoma | LTK/FAM150B | 13% |

These results suggest that FAM150 antagonists may be suitable for treating breast invasive carcinoma, ovarian serous cystadenocarcinoma, kidney renal clear cell carcinoma, colon adenocarcinoma, bladder urothelial carcinoma, and lung squamous cell carcinoma, among other cancers.

TABLE OF SEQUENCES

| SEQ ID NO | Description | Sequence |
|---|---|---|
| 1 | Human FAM150A | MRPLKPGAPL PALFLLALAL SPHGAHGRPR GRRGARVTDK |
| | precursor, with signal | EPKPLLFLPA AGAGRTPSGS RSAEIFPRDS NLKDKFIKHF |
| | peptide | TGPVTFSPEC SKHFHRLYYN TRECSTPAYY KRCARLLTRL |
| | | AVSPLCSQT |
| 2 | Human mature | RPR GRRGARVTDK EPKPLLFLPA AGAGRTPSGS |
| | FAM150A, without | RSAEIFPRDS NLKDKFIKHF TGPVTFSPEC SKHFHRLYYN |
| | signal peptide | TRECSTPAYY KRCARLLTRL AVSPLCSQT |
| 32 | Mouse FAM150A | MWLTKPSTPV SALLLLALAL SPPGTQGRPQ RSLAARVAEL |
| | precursor, with signal | RPELFLPVTG TRLPPRASRS TEIFPRDLTL KDKFIKHFTG |
| | peptide | PVTFSAECSK HFHRLYHNTR DCSTPAYYKR CARLLTRLAV |
| | | SPLCSQT |
| 33 | Mouse mature | RPQ RSLAARVAEL RPELFLPVTG TRLPPRASRS |
| | FAM150A, without | TEIFPRDLTL KDKFIKHFTG PVTFSAECSK HFHRLYHNTR |
| | signal peptide | DCSTPAYYKR CARLLTRLAV SPLCSQT |
| 3 | Human FAM150B | MRGPGHPLLL GLLLVLGAAG RGRGGAEPRE PADGQALLRL |
| | precursor, with signal | VVELVQELRK HHSAEHKGLQ LLGRDCALGR AEAAGLGPSP |
| | peptide | EQRVEIVPRD LRMKDKFLKH LTGPLYFSPK CSKHFHRLYH |
| | | NTRDCTIPAY YKRCARLLTR LAVSPVCMED KQ |
| 4 | Human mature | GAEPRE PADGQALLRL VVELVQELRK HHSAEHKGLQ |
| | FAM150B, without signal | LLGRDCALGR AEAAGLGPSP EQRVEIVPRD LRMKDKFLKH |
| | peptide | LTGPLYFSPK CSKHFHRLYH NTRDCTIPAY YKRCARLLTR |
| | | LAVSPVCMED KQ |
| 5 | Mouse FAM150B | MRVSGRPMLL ALLLLLTTVG DRGRAQSRGP ADRQTLLRLL |
| | precursor, with signal | VELVQELKKF HIGDSKRLQL LGESDFALGR REATDYGADQ |
| | peptide | EEQRVEIVPR DLRMKDKFLK HLTGPLYFSP KCSKHFHRLY |
| | | HNTRDCTIPA YYKRCARLLT RLAVSPMCME R |
| 6 | Mouse mature | QSRGP ADRQTLLRLL VELVQELKKF HIGDSKRLQL |
| | FAM150B, without signal | LGESDFALGR REATDYGADQ EEQRVEIVPR DLRMKDKFLK |
| | peptide | HLTGPLYFSP KCSKHFHRLY HNTRDCTIPA YYKRCARLLT |
| | | RLAVSPMCME R |
| 7 | Human LTK precursor, | MGCWGQLLVW FGAAGAILCS SPGSQETFLR SSPLPLASPS |
| | with signal peptide | PRDPKVSAPP SILEPASPLN SPGTEGSWLF STCGASGRHG |
| | | PTQTQCDGAY AGTSVVVTVG AAGQLRGVQL WRVPGPGQYL |
| | | ISAYGAAGGK GAKNHLSRAH GVFVSAIFSL GLGESLYILV |
| | | GQQGEDACPG GSPESQLVCL GESRAVEEHA AMDGSEGVPG |
| | | SRRWAGGGGG GGGATYVFRV RAGELEPLLV AAGGGGRAYL |
| | | RPRDRGRTQA SPEKLENRSE APGSGGRGGA AGGGGGWTSR |
| | | APSPQAGRSL QEGAEGGQGC SEAWATLGWA AAGGFGGGGG |
| | | ACTAGGGGGG YRGGDASETD NLWADGEDGV SFIHPSSELF |
| | | LQPLAVTENH GEVEIRRHLN CSHCPLRDCQ WQAELQLAEC |
| | | LCPEGMELAV DNVTCMDLHK PPGPLVLMVA VVATSTLSLL |
| | | MVCGVLILVK QKKWQGLQEM RLPSPELELS KLRTSAIRTA |
| | | PNPYYCQVGL GPAQSWPLPP GVTEVSPANV TLLRALGHGA |
| | | FGEVYEGLVI GLPGDSSPLQ VAIKTLPELC SPQDELDFLM |
| | | EALIISKFRH QNIVRCVGLS LRATPRLILL ELMSGGDMKS |
| | | FLRHSRPHLG QPSPLVMRDL LQLAQDIAQG CHYLEENHFI |
| | | HRDIAARNCL LSCAGPSRVA KIGDFGMARD IYRASYYRRG |
| | | DRALLPVKWM PPEAFLEGIF TSKTDSWSFG VLLWEIFSLG |
| | | YMPYPGRTNQ EVLDFVVGGG RMDPPRGCPG PVYRIMTQCW |
| | | QHEPELRPSF ASILERLQYC TQDPDVLNSL LPMELGPTPE |
| | | EEGTSGLGNR SLECLRPPQP QELSPEKLKS WGGSPLGPWL |
| | | SSGLKPLKSR GLQPQNLWNP TYRS |
| 8 | Human mature LTK, | ILCS SPGSQETFLR SSPLPLASPS PRDPKVSAPP |
| | without signal peptide | SILEPASPLN SPGTEGSWLF STCGASGRHG PTQTQCDGAY |
| | | AGTSVVVTVG AAGQLRGVQL WRVPGPGQYL ISAYGAAGGK |
| | | GAKNHLSRAH GVFVSAIFSL GLGESLYILV GQQGEDACPG |
| | | GSPESQLVCL GESRAVEEHA AMDGSEGVPG SRRWAGGGGG |
| | | GGGATYVFRV RAGELEPLLV AAGGGGRAYL RPRDRGRTQA |
| | | SPEKLENRSE APGSGGRGGA AGGGGGWTSR APSPQAGRSL |
| | | QEGAEGGQGC SEAWATLGWA AAGGFGGGGG ACTAGGGGGG |
| | | YRGGDASETD NLWADGEDGV SFIHPSSELF LQPLAVTENH |
| | | GEVEIRRHLN CSHCPLRDCQ WQAELQLAEC LCPEGMELAV |
| | | DNVTCMDLHK PPGPLVLMVA VVATSTLSLL MVCGVLILVK |
| | | QKKWQGLQEM RLPSPELELS KLRTSAIRTA PNPYYCQVGL |
| | | GPAQSWPLPP GVTEVSPANV TLLRALGHGA FGEVYEGLVI |
| | | GLPGDSSPLQ VAIKTLPELC SPQDELDFLM EALIISKFRH |
| | | QNIVRCVGLS LRATPRLILL ELMSGGDMKS FLRHSRPHLG |
| | | QPSPLVMRDL LQLAQDIAQG CHYLEENHFI HRDIAARNCL |
| | | LSCAGPSRVA KIGDFGMARD IYRASYYRRG DRALLPVKWM |
| | | PPEAFLEGIF TSKTDSWSFG VLLWEIFSLG YMPYPGRTNQ |
| | | EVLDFVVGGG RMDPPRGCPG PVYRIMTQCW QHEPELRPSF |
| | | ASILERLQYC TQDPDVLNSL LPMELGPTPE EEGTSGLGNR |
| | | SLECLRPPQP QELSPEKLKS WGGSPLGPWL SSGLKPLKSR |
| | | GLQPQNLWNP TYRS |
| 9 | Human LTK isoform 2 | MGCWGQLLVW FGAAGAILCS SPGSQETFLR SSPLPLASPS |
| | precursor, with signal | PRDPKVSAPP SILEPASPLN SPGTEGSWLF STCGASGRHG |
| | peptide | PTQTQCDGAY AGTSVVVTVG AAGQLRGVQL WRVPGPGQYL |
| | | ISAYGAAGGK GAKNHLSRAH GVFVSAIFSL GLGESLYILV |
| | | GQQGEDACPG GSPESQLVCL GESRAVEEHA AMDGSEGVPG |
| | | SRRWAGGGGG GGGATYVFRV RAGELEPLLV AAGGGGRAYL |
| | | RPRDRGRTQA SPEKLENRSE APGSGGRGGA AGGDASETDN |
| | | LWADGEDGVS FIHPSSELFL QPLAVTENHG EVEIRRHLNC |
| | | SHCPLRDCQW QAELQLAECL CPEGMELAVD NVTCMDLHKP |
| | | PGPLVLMVAV VATSTLSLLM VCGVLILVKQ KKWQGLQEMR |
| | | LPSPELELSK LRTSAIRTAP NPYYCQVGLG PAQSWPLPPG |
| | | VTEVSPANVT LLRALGHGAF GEVYEGLVIG LPGDSSPLQV |
| | | AIKTLPELCS PQDELDFLME ALIISKFRHQ NIVRCVGLSL |
| | | RATPRLILLE LMSGGDMKSF LRHSRPHLGQ PSPLVMRDLL |
| | | QLAQDIAQGC HYLEENHFIH RDIAARNCLL SCAGPSRVAK |
| | | IGDFGMARDI YRASYYRRGD RALLPVKWMP PEAFLEGIFT |
| | | SKTDSWSFGV LLWEIFSLGY MPYPGRTNQE VLDFVVGGGR |
| | | MDPPRGCPGP VYRIMTQCWQ HEPELRPSFA SILERLQYCT |
| | | QDPDVLNSLL PMELGPTPEE EGTSGLGNRS LECLRPPQPQ |
| | | ELSPEKLKSW GGSPLGPWLS SGLKPLKSRG LQPQNLWNPT |
| | | YRS |
| 10 | Human mature LTK | ILCS SPGSQETFLR SSPLPLASPS PRDPKVSAPP |
| | isoform 2, without signal | SILEPASPLN SPGTEGSWLF STCGASGRHG PTQTQCDGAY |
| | peptide | AGTSVVVTVG AAGQLRGVQL WRVPGPGQYL ISAYGAAGGK |
| | | GAKNHLSRAH GVFVSAIFSL GLGESLYILV GQQGEDACPG |
| | | GSPESQLVCL GESRAVEEHA AMDGSEGVPG SRRWAGGGGG |
| | | GGGATYVFRV RAGELEPLLV AAGGGGRAYL RPRDRGRTQA |
| | | SPEKLENRSE APGSGGRGGA AGGDASETDN LWADGEDGVS |
| | | FIHPSSELFL QPLAVTENHG EVEIRRHLNC SHCPLRDCQW |
| | | QAELQLAECL CPEGMELAVD NVTCMDLHKP PGPLVLMVAV |
| | | VATSTLSLLM VCGVLILVKQ KKWQGLQEMR LPSPELELSK |
| | | LRTSAIRTAP NPYYCQVGLG PAQSWPLPPG VTEVSPANVT |
| | | LLRALGHGAF GEVYEGLVIG LPGDSSPLQV AIKTLPELCS |
| | | PQDELDFLME ALIISKFRHQ NIVRCVGLSL RATPRLILLE |
| | | LMSGGDMKSF LRHSRPHLGQ PSPLVMRDLL QLAQDIAQGC |
| | | HYLEENHFIH RDIAARNCLL SCAGPSRVAK IGDFGMARDI |
| | | YRASYYRRGD RALLPVKWMP PEAFLEGIFT SKTDSWSFGV |
| | | LLWEIFSLGY MPYPGRTNQE VLDFVVGGGR MDPPRGCPGP |
| | | VYRIMTQCWQ HEPELRPSFA SILERLQYCT QDPDVLNSLL |
| | | PMELGPTPEE EGTSGLGNRS LECLRPPQPQ ELSPEKLKSW |
| | | GGSPLGPWLS SGLKPLKSRG LQPQNLWNPT YRS |
| 11 | Mouse LTK precursor, | MGCSHRLLLW LGAAGTILCS NSEFQAPFLT PSLLPVLVLN |
| | with signal peptide | SQEQKVTPTP SKLEPASLPN PLGTRGPWVF NTCGASGRSG |
| | | PTQTQCDGAY TGSSVMVTVG AAGPLKGVQL WRAPDTGQYL |
| | | ISAYGAAGGK GAQNHLSRAH GIFLSAVFFL RRGEPVYILV |
| | | GQQGQDACPG GSPESQLVCL GESGEHATTY GTERIPGWRR |
| | | WAGGGGGGGG ATSIFRLRAG EPEPLLVAAG GGGRSYRRRP |
| | | DRGRTQAVPE RLETRAAAPG SGGRGGAAGG GSGWTSRAHS |
| | | PQAGRSPREG AEGGEGCAEA WAALRWAAAG GFGGGGGACA |
| | | AGGGGGGYRG GDTSESDLLW ADGEDGTSFV HPSGELYLQP |
| | | LAVTEGHGEV EIRKHPNCSH CPFKDCQWQA ELWTAECTCP |
| | | EGTELAVDNV TCMDLPTTAS PLILMGAVVA ALALSLLMMC |
| | | AVLILVNQKC QGLWGTRLPG PELELSKLRS SAIRTAPNPY |
| | | YCQVGLSPAQ PWPLPPGLTE VSPANVTLLR ALGHGAFGEV |
| | | YEGLVTGLPG DSSPLPVAIK TLPELCSHQD ELDFLMEALI |
| | | ISKFSHQNIV RCVGLSFRSA PRLILLELMS GGDMKSFLRH |
| | | SRPHPGQLAP LTMQDLLQLA QDIAQGCHYL EENHFIHRDI |
| | | AARNCLLSCS GASRVAKIGD FGMARDIYQA SYYRKGGRTL |
| | | LPVKWMPPEA LLEGLFTSKT DSWSFGVLLW EIFSLGYMPY |
| | | PGHTNQEVLD FIATGNRMDP PRNCPGPVYR IMTQCWQHQP |
| | | ELRPDFGSIL ERIQYCTQDP DVLNSPLPME PGPILEEEEA |
| | | SRLGNRSLEG LRSPKPLELS SQNLKSWGGG LLGSWLPSGL |
| | | KTLKPRCLQP QNIWNPTYGS WTPRGPQGED TGIEQCNGSS |
| | | SSSIPGIQ |
| 12 | Mouse mature LTK, | ILCS NSEFQAPFLT PSLLPVLVLN SQEQKVTPTP |
| | without signal peptide | SKLEPASLPN PLGTRGPWVF NTCGASGRSG PTQTQCDGAY |
| | | TGSSVMVTVG AAGPLKGVQL WRAPDTGQYL ISAYGAAGGK |
| | | GAQNHLSRAH GIFLSAVFFL RRGEPVYILV GQQGQDACPG |
| | | GSPESQLVCL GESGEHATTY GTERIPGWRR WAGGGGGGGG |
| | | ATSIFRLRAG EPEPLLVAAG GGGRSYRRRP DRGRTQAVPE |
| | | RLETRAAAPG SGGRGGAAGG GSGWTSRAHS PQAGRSPREG |
| | | AEGGEGCAEA WAALRWAAAG GFGGGGGACA AGGGGGGYRG |
| | | GDTSESDLLW ADGEDGTSFV HPSGELYLQP LAVTEGHGEV |
| | | EIRKHPNCSH CPFKDCQWQA ELWTAECTCP EGTELAVDNV |
| | | TCMDLPTTAS PLILMGAVVA ALALSLLMMC AVLILVNQKC |
| | | QGLWGTRLPG PELELSKLRS SAIRTAPNPY YCQVGLSPAQ |
| | | PWPLPPGLTE VSPANVTLLR ALGHGAFGEV YEGLVTGLPG |
| | | DSSPLPVAIK TLPELCSHQD ELDFLMEALI ISKFSHQNIV |
| | | RCVGLSFRSA PRLILLELMS GGDMKSFLRH SRPHPGQLAP |
| | | LTMQDLLQLA QDIAQGCHYL EENHFIHRDI AARNCLLSCS |
| | | GASRVAKIGD FGMARDIYQA SYYRKGGRTL LPVKWMPPEA |
| | | LLEGLFTSKT DSWSFGVLLW EIFSLGYMPY PGHTNQEVLD |
| | | FIATGNRMDP PRNCPGPVYR IMTQCWQHQP ELRPDFGSIL |
| | | ERIQYCTQDP DVLNSPLPME PGPILEEEEA SRLGNRSLEG |
| | | LRSPKPLELS SQNLKSWGGG LLGSWLPSGL KTLKPRCLQP |
| | | QNIWNPTYGS WTPRGPQGED TGIEQCNGSS SSSIPGIQ |
| 13 | Human LTK ECD, to aa | MGCWGQLLVW FGAAGAILCS SPGSQETFLR SSPLPLASPS |
| | 424, with signal peptide | PRDPKVSAPP SILEPASPLN SPGTEGSWLF STCGASGRHG |
| | | PTQTQCDGAY AGTSVVVTVG AAGQLRGVQL WRVPGPGQYL |
| | | ISAYGAAGGK GAKNHLSRAH GVFVSAIFSL GLGESLYILV |
| | | GQQGEDACPG GSPESQLVCL GESRAVEEHA AMDGSEGVPG |
| | | SRRWAGGGGG GGGATYVFRV RAGELEPLLV AAGGGGRAYL |
| | | RPRDRGRTQA SPEKLENRSE APGSGGRGGA AGGGGGWTSR |
| | | APSPQAGRSL QEGAEGGQGC SEAWATLGWA AAGGFGGGGG |
| | | ACTAGGGGGG YRGGDASETD NLWADGEDGV SFIHPSSELF |
| | | LQPLAVTENH GEVEIRRHLN CSHCPLRDCQ WQAELQLAEC |
| | | LCPEGMELAV DNVTCMDLHK PPGP |
| 14 | Human LTK ECD, to aa | ILCS SPGSQETFLR SSPLPLASPS PRDPKVSAPP |
| | 424, without signal | SILEPASPLN SPGTEGSWLF STCGASGRHG PTQTQCDGAY |
| | peptide | AGTSVVVTVG AAGQLRGVQL WRVPGPGQYL ISAYGAAGGK |
| | | GAKNHLSRAH GVFVSAIFSL GLGESLYILV GQQGEDACPG |
| | | GSPESQLVCL GESRAVEEHA AMDGSEGVPG SRRWAGGGGG |
| | | GGGATYVFRV RAGELEPLLV AAGGGGRAYL RPRDRGRTQA |
| | | SPEKLENRSE APGSGGRGGA AGGGGGWTSR APSPQAGRSL |
| | | QEGAEGGQGC SEAWATLGWA AAGGFGGGGG ACTAGGGGGG |
| | | YRGGDASETD NLWADGEDGV SFIHPSSELF LQPLAVTENH |
| | | GEVEIRRHLN CSHCPLRDCQ WQAELQLAEC LCPEGMELAV |
| | | DNVTCMDLHK PPGP |
| 30 | Human LTK isoform 2 | MGCWGQLLVW FGAAGAILCS SPGSQETFLR SSPLPLASPS |
| | ECD, with signal peptide | PRDPKVSAPP SILEPASPLN SPGTEGSWLF STCGASGRHG |
| | | PTQTQCDGAY AGTSVVVTVG AAGQLRGVQL WRVPGPGQYL |
| | | ISAYGAAGGK GAKNHLSRAH GVFVSAIFSL GLGESLYILV |
| | | GQQGEDACPG GSPESQLVCL GESRAVEEHA AMDGSEGVPG |
| | | SRRWAGGGGG GGGATYVFRV RAGELEPLLV AAGGGGRAYL |
| | | RPRDRGRTQA SPEKLENRSE APGSGGRGGA AGGDASETDN |
| | | LWADGEDGVS FIHPSSELFL QPLAVTENHG EVEIRRHLNC |
| | | SHCPLRDCQW QAELQLAECL CPEGMELAVD NVTCMDLHKP |
| | | PGP |
| 31 | Human mature LTK | ILCS SPGSQETFLR SSPLPLASPS PRDPKVSAPP |
| | isoform 2 ECD, without | SILEPASPLN SPGTEGSWLF STCGASGRHG PTQTQCDGAY |
| | signal peptide | AGTSVVVTVG AAGQLRGVQL WRVPGPGQYL ISAYGAAGGK |
| | | GAKNHLSRAH GVFVSAIFSL GLGESLYILV GQQGEDACPG |
| | | GSPESQLVCL GESRAVEEHA AMDGSEGVPG SRRWAGGGGG |
| | | GGGATYVFRV RAGELEPLLV AAGGGGRAYL RPRDRGRTQA |
| | | SPEKLENRSE APGSGGRGGA AGGDASETDN LWADGEDGVS |
| | | FIHPSSELFL QPLAVTENHG EVEIRRHLNC SHCPLRDCQW |
| | | QAELQLAECL CPEGMELAVD NVTCMDLHKP PGP |
| 15 | Mouse LTK ECD, to aa | MGCSHRLLLW LGAAGTILCS NSEFQAPFLT PSLLPVLVLN |
| | 421, with signal peptide | SQEQKVTPTP SKLEPASLPN PLGTRGPWVF NTCGASGRSG |
| | | PTQTQCDGAY TGSSVMVTVG AAGPLKGVQL WRAPDTGQYL |
| | | ISAYGAAGGK GAQNHLSRAH GIFLSAVFFL RRGEPVYILV |
| | | GQQGQDACPG GSPESQLVCL GESGEHATTY GTERIPGWRR |
| | | WAGGGGGGGG ATSIFRLRAG EPEPLLVAAG GGGRSYRRRP |
| | | DRGRTQAVPE RLETRAAAPG SGGRGGAAGG GSGWTSRAHS |
| | | PQAGRSPREG AEGGEGCAEA WAALRWAAAG GFGGGGGACA |
| | | AGGGGGGYRG GDTSESDLLW ADGEDGTSFV HPSGELYLQP |
| | | LAVTEGHGEV EIRKHPNCSH CPFKDCQWQA ELWTAECTCP |
| | | EGTELAVDNV TCMDLPTTAS P |
| 16 | Mouse LTK ECD, to aa | ILCS NSEFQAPFLT PSLLPVLVLN SQEQKVTPTP |
| | 421, without signal | SKLEPASLPN PLGTRGPWVF NTCGASGRSG PTQTQCDGAY |
| | peptide | TGSSVMVTVG AAGPLKGVQL WRAPDTGQYL ISAYGAAGGK |
| | | GAQNHLSRAH GIFLSAVFFL RRGEPVYILV GQQGQDACPG |
| | | GSPESQLVCL GESGEHATTY GTERIPGWRR WAGGGGGGGG |
| | | ATSIFRLRAG EPEPLLVAAG GGGRSYRRRP DRGRTQAVPE |
| | | RLETRAAAPG SGGRGGAAGG GSGWTSRAHS PQAGRSPREG |
| | | AEGGEGCAEA WAALRWAAAG GFGGGGGACA AGGGGGGYRG |
| | | GDTSESDLLW ADGEDGTSFV HPSGELYLQP LAVTEGHGEV |
| | | EIRKHPNCSH CPFKDCQWQA ELWTAECTCP EGTELAVDNV |
| | | TCMDLPTTAS P |
| 17 | Fc C237S | EPKSSDKTHT CPPCPAPELL GGPSVFLFPP KPKDTLMISR |
| | | TPEVTCVVVD VSHEDPEVKF NWYVDGVEVH NAKTKPREEQ |
| | | YNSTYRVVSV LTVLHQDWLN GKEYKCKVSN KALPAPIEKT |
| | | ISKAKGQPRE PQVYTLPPSR DELTKNQVSL TCLVKGFYPS |
| | | DIAVEWESNG QPENNYKTTP PVLDSDGSFF LYSKLTVDKS |
| | | RWQQGNVFSC SVMHEALHNH YTQKSLSLSP GK |
| 18 | Exemplary Fc #1 | ERKCCVECPP CPAPPVAGPS VFLFPPKPKD TLMISRTPEV |
| | | TCVVVDVSHE DPEVQFNWYV DGVEVHNAKT KPREEQFNST |
| | | FRVVSVLTVV HQDWLNGKEY KCKVSNKGLP APIEKTISKT |
| | | KGQPREPQVY TLPPSREEMT KNQVSLTCLV KGFYPSDIAV |
| | | EWESNGQPEN NYKTTPPMLD SDGSFFLYSK LTVDKSRWQQ |
| | | GNVFSCSVMH EALHNHYTQK SLSLSPGK |
| 19 | Exemplary Fc #2 | ESKYGPPCPS CPAPEFLGGP SVFLFPPKPK DTLMISRTPE |
| | | VTCVVVDVSQ EDPEVQFNWY VDGVEVHNAK TKPREEQFNS |
| | | TYRVVSVLTV LHQDWLNGKE YKCKVSNKGL PSSIEKTISK |
| | | AKGQPREPQV YTLPPSQEEM TKNQVSLTCL VKGFYPSDIA |
| | | VEWESNGQPE NNYKTTPPVL DSDGSFFLYS RLTVDKSRWQ |
| | | EGNVFSCSVM HEALHNHYTQ KSLSLSLGK |
| 20 | Human ALK precursor, | MGAIGLLWLL PLLLSTAAVG SGMGTGQRAG SPAAGPPLQP |
| | with signal sequence | REPLSYSRLQ RKSLAVDFVV PSLFRVYARD LLLPPSSSEL |
| | | KAGRPEARGS LALDCAPLLR LLGPAPGVSW TAGSPAPAEA |
| | | RTLSRVLKGG SVRKLRRAKQ LVLELGEEAI LEGCVGPPGE |
| | | AAVGLLQFNL SELFSWWIRQ GEGRLRIRLM PEKKASEVGR |
| | | EGRLSAAIRA SQPRLLFQIF GTGHSSLESP TNMPSPSPDY |
| | | FTWNLTWIMK DSFPFLSHRS RYGLECSFDF PCELEYSPPL |
| | | HDLRNQSWSW RRIPSEEASQ MDLLDGPGAE RSKEMPRGSF |
| | | LLLNTSADSK HTILSPWMRS SSEHCTLAVS VHRHLQPSGR |
| | | YIAQLLPHNE AAREILLMPT PGKHGWTVLQ GRIGRPDNPF |
| | | RVALEYISSG NRSLSAVDFF ALKNCSEGTS PGSKMALQSS |
| | | FTCWNGTVLQ LGQACDFHQD CAQGEDESQM CRKLPVGFYC |
| | | NFEDGFCGWT QGILSPHIPQ WQVRTLKDAR FQDHQDHALL |
| | | LSTTDVPASE SATVTSATFP APIKSSPCEL RMSWLIRGVL |
| | | RGNVSLVLVE NKTGKEQGRM VWHVAAYEGL SLWQWMVLPL |
| | | LDVSDRFWLQ MVAWWGQGSR AIVAFDNISI SLDCYLTISG |
| | | EDKILQNTAP KSRNLFERNP NKELKPGENS PRQTPIFDPT |
| | | VHWLFTTCGA SGPHGPTQAQ CNNAYQNSNL SVEVGSEGPL |
| | | KGIQIWKVPA TDTYSISGYG AAGGKGGKNT MMRSHGVSVL |
| | | GIFNLEKDDM LYILVGQQGE DACPSTNQLI QKVCIGENNV |
| | | IEEEIRVNRS VHEWAGGGGG GGGATYVFKM KDGVPVPLII |
| | | AAGGGGRAYG AKTDTFHPER LENNSSVLGL NGNSGAAGGG |
| | | GGWNDNTSLL WAGKSLQEGA TGGHSCPQAM KKWGWETRGG |
| | | FGGGGGGCSS GGGGGGYIGG NAASNNDPEM DGEDGVSFIS |
| | | PLGILYTPAL KVMEGHGEVN IKHYLNCSHC EVDECHMDPE |
| | | SHKVICFCDH GTVLAEDGVS CIVSPTPEPH LPLSLILSVV |
| | | TSALVAALVL AFSGIMIVYR RKHQELQAMQ MELQSPEYKL |
| | | SKLRTSTIMT DYNPNYCFAG KTSSISDLKE VPRKNITLIR |
| | | GLGHGAFGEV YEGQVSGMPN DPSPLQVAVK TLPEVCSEQD |
| | | ELDFLMEALI ISKFNHQNIV RCIGVSLQSL PRFILLELMA |
| | | GGDLKSFLRE TRPRPSQPSS LAMLDLLHVA RDIACGCQYL |
| | | EENHFIHRDI AARNCLLTCP GPGRVAKIGD FGMARDIYRA |
| | | SYYRKGGCAM LPVKWMPPEA FMEGIFTSKT DTWSFGVLLW |
| | | EIFSLGYMPY PSKSNQEVLE FVTSGGRMDP PKNCPGPVYR |
| | | IMTQCWQHQP EDRPNFAIIL ERIEYCTQDP DVINTALPIE |
| | | YGPLVEEEEK VPVRPKDPEG VPPLLVSQQA KREEERSPAA |
| | | PPPLPTTSSG KAAKKPTAAE ISVRVPRGPA VEGGHVNMAF |
| | | SQSNPPSELH KVHGSRNKPT SLWNPTYGSW FTEKPTKKNN |
| | | PIAKKEPHDR GNLGLEGSCT VPPNVATGRL PGASLLLEPS |
| | | SLTANMKEVP LFRLRHFPCG NVNYGYQQQG LPLEAATAPG |
| | | AGHYEDTILK SKNSMNQPGP |
| 21 | Human ALK, without | VG SGMGTGQRAG SPAAGPPLQP REPLSYSRLQ |
| | signal sequence | RKSLAVDFVV PSLFRVYARD LLLPPSSSEL KAGRPEARGS |
| | | LALDCAPLLR LLGPAPGVSW TAGSPAPAEA RTLSRVLKGG |
| | | SVRKLRRAKQ LVLELGEEAI LEGCVGPPGE AAVGLLQFNL |
| | | SELFSWWIRQ GEGRLRIRLM PEKKASEVGR EGRLSAAIRA |
| | | SQPRLLFQIF GTGHSSLESP TNMPSPSPDY FTWNLTWIMK |
| | | DSFPFLSHRS RYGLECSFDF PCELEYSPPL HDLRNQSWSW |
| | | RRIPSEEASQ MDLLDGPGAE RSKEMPRGSF LLLNTSADSK |
| | | HTILSPWMRS SSEHCTLAVS VHRHLQPSGR YIAQLLPHNE |
| | | AAREILLMPT PGKHGWTVLQ GRIGRPDNPF RVALEYISSG |
| | | NRSLSAVDFF ALKNCSEGTS PGSKMALQSS FTCWNGTVLQ |
| | | LGQACDFHQD CAQGEDESQM CRKLPVGFYC NFEDGFCGWT |
| | | QGILSPHIPQ WQVRTLKDAR FQDHQDHALL LSTTDVPASE |
| | | SATVTSATFP APIKSSPCEL RMSWLIRGVL RGNVSLVLVE |
| | | NKTGKEQGRM VWHVAAYEGL SLWQWMVLPL LDVSDRFWLQ |
| | | MVAWWGQGSR AIVAFDNISI SLDCYLTISG EDKILQNTAP |
| | | KSRNLFERNP NKELKPGENS PRQTPIFDPT VHWLFTTCGA |
| | | SGPHGPTQAQ CNNAYQNSNL SVEVGSEGPL KGIQIWKVPA |
| | | TDTYSISGYG AAGGKGGKNT MMRSHGVSVL GIFNLEKDDM |
| | | LYILVGQQGE DACPSTNQLI QKVCIGENNV IEEEIRVNRS |
| | | VHEWAGGGGG GGGATYVFKM KDGVPVPLII AAGGGGRAYG |
| | | AKTDTFHPER LENNSSVLGL NGNSGAAGGG GGWNDNTSLL |
| | | WAGKSLQEGA TGGHSCPQAM KKWGWETRGG FGGGGGGCSS |
| | | GGGGGGYIGG NAASNNDPEM DGEDGVSFIS PLGILYTPAL |
| | | KVMEGHGEVN IKHYLNCSHC EVDECHMDPE SHKVICFCDH |
| | | GTVLAEDGVS CIVSPTPEPH LPLSLILSVV TSALVAALVL |
| | | AFSGIMIVYR RKHQELQAMQ MELQSPEYKL SKLRTSTIMT |
| | | DYNPNYCFAG KTSSISDLKE VPRKNITLIR GLGHGAFGEV |
| | | YEGQVSGMPN DPSPLQVAVK TLPEVCSEQD ELDFLMEALI |
| | | ISKFNHQNIV RCIGVSLQSL PRFILLELMA GGDLKSFLRE |
| | | TRPRPSQPSS LAMLDLLHVA RDIACGCQYL EENHFIHRDI |
| | | AARNCLLTCP GPGRVAKIGD FGMARDIYRA SYYRKGGCAM |
| | | LPVKWMPPEA FMEGIFTSKT DTWSFGVLLW EIFSLGYMPY |
| | | PSKSNQEVLE FVTSGGRMDP PKNCPGPVYR IMTQCWQHQP |
| | | EDRPNFAIIL ERIEYCTQDP DVINTALPIE YGPLVEEEEK |
| | | VPVRPKDPEG VPPLLVSQQA KREEERSPAA PPPLPTTSSG |
| | | KAAKKPTAAE ISVRVPRGPA VEGGHVNMAF SQSNPPSELH |
| | | KVHGSRNKPT SLWNPTYGSW FTEKPTKKNN PIAKKEPHDR |
| | | GNLGLEGSCT VPPNVATGRL PGASLLLEPS SLTANMKEVP |
| | | LFRLRHFPCG NVNYGYQQQG LPLEAATAPG AGHYEDTILK |
| | | SKNSMNQPGP |
| 22 | Human LTK ECD-Fc, | MGCWGQLLVW FGAAGAILCS SPGSQETFLR SSPLPLASPS |
| | with signal sequence | PRDPKVSAPP SILEPASPLN SPGTEGSWLF STCGASGRHG |
| | | PTQTQCDGAY AGTSVVVTVG AAGQLRGVQL WRVPGPGQYL |
| | | ISAYGAAGGK GAKNHLSRAH GVFVSAIFSL GLGESLYILV |
| | | GQQGEDACPG GSPESQLVCL GESRAVEEHA AMDGSEGVPG |
| | | SRRWAGGGGG GGGATYVFRV RAGELEPLLV AAGGGGRAYL |
| | | RPRDRGRTQA SPEKLENRSE APGSGGRGGA AGGGGGWTSR |
| | | APSPQAGRSL QEGAEGGQGC SEAWATLGWA AAGGFGGGGG |
| | | ACTAGGGGGG YRGGDASETD NLWADGEDGV SFIHPSSELF |
| | | LQPLAVTENH GEVEIRRHLN CSHCPLRDCQ WQAELQLAEC |
| | | LCPEGMELAV DNVTCMDLHK PPGPLGSEPK SSDKTHTCPP |
| | | CPAPELLGGP SVFLFPPKPK DTLMISRTPE VTCVVVDVSH |
| | | EDPEVKFNWY VDGVEVHNAK TKPREEQYNS TYRVVSVLTV |
| | | LHQDWLNGKE YKCKVSNKAL PAPIEKTISK AKGQPREPQV |
| | | YTLPPSRDEL TKNQVSLTCL VKGFYPSDIA VEWESNGQPE |
| | | NNYKTTPPVL DSDGSFFLYS KLTVDKSRWQ QGNVFSCSVM |
| | | HEALHNHYTQ KSLSLSPGK |
| 23 | Human LTK ECD-Fc, | ILCSSPGSQE TFLRSSPLPL ASPSPRDPKV SAPPSILEPA |
| | without signal sequence | SPLNSPGTEG SWLFSTCGAS GRHGPTQTQC DGAYAGTSVV |
| | | VTVGAAGQLR GVQLWRVPGP GQYLISAYGA AGGKGAKNHL |
| | | SRAHGVFVSA IFSLGLGESL YILVGQQGED ACPGGSPESQ |
| | | LVCLGESRAV EEHAAMDGSE GVPGSRRWAG GGGGGGGATY |
| | | VFRVRAGELE PLLVAAGGGG RAYLRPRDRG RTQASPEKLE |
| | | NRSEAPGSGG RGGAAGGGGG WTSRAPSPQA GRSLQEGAEG |
| | | GQGCSEAWAT LGWAAAGGFG GGGGACTAGG GGGGYRGGDA |
| | | SETDNLWADG EDGVSFIHPS SELFLQPLAV TENHGEVEIR |
| | | RHLNCSHCPL RDCQWQAELQ LAECLCPEGM ELAVDNVTCM |
| | | DLHKPPGPLG SEPKSSDKTH TCPPCPAPEL LGGPSVFLFP |
| | | PKPKDTLMIS RIPEVICVVV DVSHEDPEVK FNWYVDGVEV |
| | | HNAKTKPREE QYNSTYRVVS VLTVLHQDWL NGKEYKCKVS |
| | | NKALPAPIEK TISKAKGQPR EPQVYTLPPS RDELTKNQVS |
| | | LTCLVKGFYP SDIAVEWESN GQPENNYKTT PPVLDSDGSF |
| | | FLYSKLTVDK SRWQQGNVFS CSVMHEALHN HYTQKSLSLS |
| | | PGK |
| 24 | Human ALK ECD-Fc, | MGAIGLLWLL PLLLSTAAVG SGMGTGQRAG SPAAGSPLQP |
| | with signal sequence | REPLSYSRLQ RKSLAVDFVV PSLFRVYARD LLLPPSSSEL |
| | | KAGRPEARGS LALDCAPLLR LLGPAPGVSW TAGSPAPAEA |
| | | RTLSRVLKGG SVRKLRRAKQ LVLELGEEAI LEGCVGPPGE |
| | | AAVGLLQFNL SELFSWWIRQ GEGRLRIRLM PEKKASEVGR |
| | | EGRLSAAIRA SQPRLLFQIF GTGHSSLESP TNMPSPSPDY |
| | | FTWNLTWIMK DSFPFLSHRS RYGLECSFDF PCELEYSPPL |
| | | HDLRNQSWSW RRIPSEEASQ MDLLDGPGAE RSKEMPRGSF |
| | | LLLNTSADSK HTILSPWMRS SSEHCTLAVS VHRHLQPSGR |
| | | YIAQLLPHNE AAREILLMPT PGKHGWTVLQ GRIGRPDNPF |
| | | RVALEYISSG NRSLSAVDFF ALKNCSEGTS PGSKMALQSS |
| | | FTCWNGTVLQ LGQACDFHQD CAQGEDESQM CRKLPVGFYC |
| | | NFEDGFCGWT QGILSPHIPQ WQVRTLKDAR FQDHQDHALL |
| | | LSTTDVPASE SATVTSATFP APIKSSPCEL RMSWLIRGVL |
| | | RGNVSLVLVE NKTGKEQGRM VWHVAAYEGL SLWQWMVLPL |
| | | LDVSDRFWLQ MVAWWGQGSR AIVAFDNISI SLDCYLTISG |
| | | EDKILQNTAP KSRNLFERNP NKELKPGENS PRQTPIFDPT |
| | | VHWLFTTCGA SGPHGPTQAQ CNNAYQNSNL SVEVGSEGPL |
| | | KGIQIWKVPA TDTYSISGYG AAGGKGGKNT MMRSHGVSVL |
| | | GIFNLEKDDM LYILVGQQGE DACPSTNQLI QKVCIGENNV |
| | | IEEEIRVNRS VHEWAGGGGG GGGATYVFKM KDGVPVPLII |
| | | AAGGGGRAYG AKTDTFHPER LENNSSVLGL NGNSGAAGGG |
| | | GGWNDNTSLL WAGKSLQEGA TGGHSCPQAM KKWGWETRGG |
| | | FGGGGGGCSS GGGGGGYIGG NAASNNDPEM DGEDGVSFIS |
| | | PLGILYTPAL KVMEGHGEVN IKHYLNCSHC EVDECHMDPE |
| | | SHKVICFCDH GTVLAEDGVS CIVSPTPEPH LPLSLILSGS |
| | | EPKSSDKTHT CPPCPAPELL GGPSVFLFPP KPKDTLMISR |
| | | TPEVTCVVVD VSHEDPEVKF NWYVDGVEVH NAKTKPREEQ |
| | | YNSTYRVVSV LTVLHQDWLN GKEYKCKVSN KALPAPIEKT |
| | | ISKAKGQPRE PQVYTLPPSR DELTKNQVSL TCLVKGFYPS |
| | | DIAVEWESNG QPENNYKTTP PVLDSDGSFF LYSKLTVDKS |
| | | RWQQGNVFSC SVMHEALHNH YTQKSLSLSP GK |
| 25 | Human ALK ECD-Fc, | VGSGMGTGQR AGSPAAGSPL QPREPLSYSR LQRKSLAVDF |
| | without signal sequence | VVPSLFRVYA RDLLLPPSSS ELKAGRPEAR GSLALDCAPL |
| | | LRLLGPAPGV SWTAGSPAPA EARTLSRVLK GGSVRKLRRA |
| | | KQLVLELGEE AILEGCVGPP GEAAVGLLQF NLSELFSWWI |
| | | RQGEGRLRIR LMPEKKASEV GREGRLSAAI RASQPRLLFQ |
| | | IFGTGHSSLE SPTNMPSPSP DYFTWNLTWI MKDSFPFLSH |
| | | RSRYGLECSF DFPCELEYSP PLHDLRNQSW SWRRIPSEEA |
| | | SQMDLLDGPG AERSKEMPRG SFLLLNTSAD SKHTILSPWM |
| | | RSSSEHCTLA VSVHRHLQPS GRYIAQLLPH NEAAREILLM |
| | | PTPGKHGWTV LQGRIGRPDN PFRVALEYIS SGNRSLSAVD |
| | | FFALKNCSEG TSPGSKMALQ SSFTCWNGTV LQLGQACDFH |
| | | QDCAQGEDES QMCRKLPVGF YCNFEDGFCG WTQGILSPHT |
| | | PQWQVRTLKD ARFQDHQDHA LLLSTTDVPA SESATVTSAT |
| | | FPAPIKSSPC ELRMSWLIRG VLRGNVSLVL VENKTGKEQG |
| | | RMVWHVAAYE GLSLWQWMVL PLLDVSDRFW LQMVAWWGQG |
| | | SRAIVAFDNI SISLDCYLTI SGEDKILQNT APKSRNLFER |
| | | NPNKELKPGE NSPRQTPIFD PTVHWLFTTC GASGPHGPTQ |
| | | AQCNNAYQNS NLSVEVGSEG PLKGIQIWKV PATDTYSISG |
| | | YGAAGGKGGK NTMMRSHGVS VLGIFNLEKD DMLYILVGQQ |
| | | GEDACPSTNQ LIQKVCIGEN NVIEEEIRVN RSVHEWAGGG |
| | | GGGGGATYVF KMKDGVPVPL IIAAGGGGRA YGAKTDTFHP |
| | | ERLENNSSVL GLNGNSGAAG GGGGWNDNTS LLWAGKSLQE |
| | | GATGGHSCPQ AMKKWGWETR GGFGGGGGGC SSGGGGGGYI |
| | | GGNAASNNDP EMDGEDGVSF ISPLGILYTP ALKVMEGHGE |
| | | VNIKHYLNCS HCEVDECHMD PESHKVICFC DHGTVLAEDG |
| | | VSCIVSPTPE PHLPLSLILS GSEPKSSDKT HTCPPCPAPE |
| | | LLGGPSVFLF PPKPKDTLMI SRIPEVICVV VDVSHEDPEV |
| | | KFNWYVDGVE VHNAKTKPRE EQYNSTYRVV SVLTVLHQDW |
| | | LNGKEYKCKV SNKALPAPIE KTISKAKGQP REPQVYTLPP |
| | | SRDELTKNQV SLTCLVKGFY PSDIAVEWES NGQPENNYKT |
| | | TPPVLDSDGS FFLYSKLTVD KSRWQQGNVF SCSVMHEALH |
| | | NHYTQKSLSL SPGK |
| 26 | Mouse LTK ECD-Fc, | MGCSHRLLLW LGAAGTILCS NSEFQTPFLT PSLLPVLVLN |
| | with signal sequence | SQEQKVTPTP SKLEPASLPN PLGTRGPWVF NTCGASGRSG |
| | (human Fc) | PTQTQCDGAY TGSSVMVTVG AAGPLKGVQL WRVPDTGQYL |
| | | ISAYGAAGGK GAQNHLSRAH GIFLSAVFFL RRGEPVYILV |
| | | GQQGQDACPG GSPESQLVCL GESGEHATTY GTERIPGWRR |
| | | WAGGGGGGGG ATSIFRLRAG EPEPLLVAAG GGGRSYRRRP |
| | | DRGRTQAVPE RLETRAAAPG SGGRGGAAGG GSGWTSRAHS |
| | | PQAGRSPREG AEGGEGCAEA WAALRWAAAG GFGGGGGACA |
| | | AGGGGGGYRG GDTSESDLLW ADGEDGTSFV HPSGELYLQP |
| | | LAVTEGHGEV EIRKHPNCSH CPFKDCQWQA ELWTAECTCP |
| | | EGTELAVDNV TCMDLPTTAS PGSEPKSSDK THTCPPCPAP |
| | | ELLGGPSVFL FPPKPKDTLM ISRTPEVTCV VVDVSHEDPE |
| | | VKFNWYVDGV EVHNAKTKPR EEQYNSTYRV VSVLTVLHQD |
| | | WLNGKEYKCK VSNKALPAPI EKTISKAKGQ PREPQVYTLP |
| | | PSRDELTKNQ VSLTCLVKGF YPSDIAVEWE SNGQPENNYK |
| | | TTPPVLDSDG SFFLYSKLTV DKSRWQQGNV FSCSVMHEAL |
| | | HNHYTQKSLS LSPGK |
| 27 | Mouse LTK ECD-Fc, | ILCSNSEFQT PFLTPSLLPV LVLNSQEQKV TPTPSKLEPA |
| | without signal sequence | SLPNPLGTRG PWVFNTCGAS GRSGPTQTQC DGAYTGSSVM |
| | (human Fc) | VTVGAAGPLK GVQLWRVPDT GQYLISAYGA AGGKGAQNHL |
| | | SRAHGIFLSA VFFLRRGEPV YILVGQQGQD ACPGGSPESQ |
| | | LVCLGESGEH ATTYGTERIP GWRRWAGGGG GGGGATSIFR |
| | | LRAGEPEPLL VAAGGGGRSY RRRPDRGRTQ AVPERLETRA |
| | | AAPGSGGRGG AAGGGSGWTS RAHSPQAGRS PREGAEGGEG |
| | | CAEAWAALRW AAAGGFGGGG GACAAGGGGG GYRGGDTSES |
| | | DLLWADGEDG TSFVHPSGEL YLQPLAVTEG HGEVEIRKHP |
| | | NCSHCPFKDC QWQAELWTAE CTCPEGTELA VDNVTCMDLP |
| | | TTASPGSEPK SSDKTHTCPP CPAPELLGGP SVFLFPPKPK |
| | | DTLMISRTPE VTCVVVDVSH EDPEVKFNWY VDGVEVHNAK |
| | | TKPREEQYNS TYRVVSVLTV LHQDWLNGKE YKCKVSNKAL |
| | | PAPIEKTISK AKGQPREPQV YTLPPSRDEL TKNQVSLTCL |
| | | VKGFYPSDIA VEWESNGQPE NNYKTTPPVL DSDGSFFLYS |
| | | KLTVDKSRWQ QGNVFSCSVM HEALHNHYTQ KSLSLSPGK |
| 28 | Human LTK (short) ECD- | MGCWGQLLVW FGAAGAILCS SPGSQETFLR SSPLPLASPS |
| | Fc, with signal sequence | PRDPKVSAPP SILEPASPLN SPGTEGSWLF STCGASGRHG |
| | | PTQTQCDGAY AGTSVVVTVG AAGQLRGVQL WRVPGPGQYL |
| | | ISAYGAAGGK GAKNHLSRAH GVFVSAIFSL GLGESLYILV |
| | | GQQGEDACPG GSPESQLVCL GESRAVEEHA AMDGSEGVPG |
| | | SRRWAGGGGG GGGATYVFRV RAGELEPLLV AAGGGGRAYL |
| | | RPRDRGRTQA SPEKLENRSE APGSGGRGGA AGGDASETDN |
| | | LWADGEDGVS FIHPSSELFL QPLAVTENHG EVEIRRHLNC |
| | | SHCPLRDCQW QAELQLAECL CPEGMELAVD NVTCMDLHKP |
| | | PGPGSEPKSS DKTHTCPPCP APELLGGPSV FLFPPKPKDT |
| | | LMISRTPEVT CVVVDVSHED PEVKFNWYVD GVEVHNAKTK |
| | | PREEQYNSTY RVVSVLTVLH QDWLNGKEYK CKVSNKALPA |
| | | PIEKTISKAK GQPREPQVYT LPPSRDELTK NQVSLTCLVK |
| | | GFYPSDIAVE WESNGQPENN YKTTPPVLDS DGSFFLYSKL |
| | | TVDKSRWQQG NVFSCSVMHE ALHNHYTQKS LSLSPGK |
| 29 | Human LTK(short) ECD- | ILCSSPGSQE TFLRSSPLPL ASPSPRDPKV SAPPSILEPA |
| | Fc, without signal | SPLNSPGTEG SWLFSTCGAS GRHGPTQTQC DGAYAGTSVV |
| | sequence | VTVGAAGQLR GVQLWRVPGP GQYLISAYGA AGGKGAKNHL |
| | | SRAHGVFVSA IFSLGLGESL YILVGQQGED ACPGGSPESQ |
| | | LVCLGESRAV EEHAAMDGSE GVPGSRRWAG GGGGGGGATY |
| | | VFRVRAGELE PLLVAAGGGG RAYLRPRDRG RTQASPEKLE |
| | | NRSEAPGSGG RGGAAGGDAS ETDNLWADGE DGVSFIHPSS |
| | | ELFLQPLAVT ENHGEVEIRR HLNCSHCPLR DCQWQAELQL |
| | | AECLCPEGME LAVDNVTCMD LHKPPGPGSE PKSSDKTHTC |
| | | PPCPAPELLG GPSVFLFPPK PKDTLMISRT PEVTCVVVDV |
| | | SHEDPEVKFN WYVDGVEVHN AKTKPREEQY NSTYRVVSVL |
| | | TVLHQDWLNG KEYKCKVSNK ALPAPIEKTI SKAKGQPREP |
| | | QVYTLPPSRD ELTKNQVSLT CLVKGFYPSD IAVEWESNGQ |
| | | PENNYKTTPP VLDSDGSFFL YSKLTVDKSR WQQGNVFSCS |
| | | VMHEALHNHY TQKSLSLSPG K |

1. A method of inhibiting ligand-induced phosphorylation of LTK in a subject comprising administering to the subject at least one molecule selected from a FAM150A antagonist, a FAM150B antagonist, and a FAM150A/B antagonist.

2. A method of inhibiting ligand-induced phosphorylation of LTK in a cell comprising contacting the cell with at least one molecule selected from a FAM150A antagonist, a FAM150B antagonist, and a FAM150A/B antagonist.

3. The method of claim 2, wherein the cell is in vitro.

4. A method of inhibiting binding of FAM150A and/or FAM150B to LTK in a subject comprising administering to the subject at least one molecule selected from a FAM150A antagonist, a FAM150B antagonist, and a FAM150A/B antagonist.

5. A method of inhibiting binding of FAM150A and/or FAM150B to LTK in a cell comprising contacting the cell with at least one molecule selected from a FAM150A antagonist, a FAM150B antagonist, and a FAM150A/B antagonist.

6. The method of claim 5, wherein the cell is in vitro.

7. A method of treating cancer comprising administering to a subject with cancer an effective amount of at least one molecule selected from a FAM150A antagonist, a FAM150B antagonist, and a FAM150A/B antagonist.

8. The method of claim 7, wherein the cancer is selected from lung cancer, leukemia, breast cancer, ovarian cancer, kidney cancer, colon cancer, and bladder cancer.

9. The method of claim 7, wherein the cancer is selected from non-small lung cancer, acute myeloid leukemia, and chronic lymphocytic leukemia.

10. The method of any one of claims 7 to 9, wherein the method further comprises administering to the subject an effective amount of a therapeutic agent selected from chemotherapeutic agents, anti-angiogenesis agents, growth inhibitory agents, and anti-neoplastic compositions.

11. A method of treating an autoimmune condition comprising administering to a subject with the autoimmune condition an effective amount of at least one molecule selected from a FAM150A antagonist, a FAM150B antagonist, and a FAM150A/B antagonist.

12. The method of claim 11, wherein the autoimmune condition is selected from rheumatoid arthritis, systemic lupus erythematosus, ankylosing spondylitis, inflammatory bowel disease, and multiple sclerosis.

13. The method of claim 11 or claim 12, wherein the method further comprises administering to the subject an effective amount of a pharmaceutical agent selected from DMARDs, TNF inhibitors and immunosuppressive agents.

14. The method of any one of the preceding claims, wherein the method comprises administering a FAM150A antagonist selected from a FAM150A antibody, a leukocyte tyrosine kinase (LTK) antibody, an LTK extracellular domain (ECD), an LTK ECD fusion molecule, and an ALK antibody.

15. The method of any one of the preceding claims, wherein the method comprises administering a FAM150B antagonist selected from a FAM150B antibody, a leukocyte tyrosine kinase (LTK) antibody, an LTK extracellular domain (ECD), an LTK ECD fusion molecule, and an ALK antibody.

16. The method of any one of the preceding claims, wherein the method comprises administering a FAM150A/B antagonist selected from a FAM150A/B antibody, a leukocyte tyrosine kinase (LTK) antibody, an LTK extracellular domain (ECD), an LTK ECD fusion molecule, and an ALK antibody.

17. The method of any one of the preceding claims, wherein the method comprises administering at least one molecule selected from a FAM150A antibody, a FAM150B antibody, and a FAM150A/B antibody.

18. The method of any one of the preceding claims, wherein the antibody is selected from a chimeric antibody, a humanized antibody, and a human antibody.

19. The method of claim any one of the preceding claims, wherein the antibody is an antibody fragment.

20. The method of claim 19, wherein the antibody fragment is selected from an Fv, a single-chain Fv (scFv), a Fab, a Fab′, and a (Fab′)₂.

21. The method of any one of claims 1 to 16, wherein the method comprises administering an LTK ECD.

22. The method of claim 21, wherein the LTK ECD comprises a sequence selected from SEQ ID NOs: 13, 14, 30, and 31.

23. The method of any one of claims 1 to 16, wherein the method comprises administering an LTK ECD fusion molecule.

24. The method of claim 23, wherein the LTK ECD fusion molecule comprises an LTK ECD and at least one fusion partner.

25. The method of claim 24, wherein at least one fusion partner is selected from an Fc, albumin, and polyethylene glycol.

26. The method of claim 24 or claim 25, wherein at least one fusion partner is an Fc.

27. The method of claim 26, wherein the Fc comprises a sequence selected from SEQ ID NOs: 17 to 19.

28. The method of claim 24 or claim 25, wherein at least one fusion partner is polyethylene glycol.

29. The method of any one of claims 23 to 28, wherein the LTK ECD portion of the LTK ECD fusion molecule comprises a sequence selected from SEQ ID NOs: 13, 14, 30, and 31.

30. A method of increasing ligand-induced phosphorylation of LTK in a subject comprising administering at least one LTK agonist to the subject.

31. A method of increasing neuronal differentiation in a subject comprising administering at least one at least one LTK agonist to the subject.

32. A method of increasing ligand-induced phosphorylation of LTK in a cell comprising contacting the cell with at least one LTK agonist.

33. The method of claim 32, wherein the cell is in vitro.

34. A method of treating a neurodegenerative disorder, comprising administering at least one at least one LTK agonist to a subject with a neurodegenerative disorder.

35. The method of claim 34, wherein the neurodegenerative disorder is selected from Huntington's disease, Parkinson's disease, and Alzheimer's disease.

36. The method of claim 35, wherein the method further comprises administering a therapeutic agent selected from cholinesterase inhibitors, such as donepezil (Aricept®), galantamine (Razadyne®), and rivastigmine (Exelon®); memantine (Namenda®); tetrabenazine (Xenazine®), antipsychotic agents, such as haloperidol (Haldol®) and clozapine, clonazepam (Klonapin®), and diazepam; antidepressants, such as escitalopram (Lexapro®), fluoxetine (Prozac®, Sarafem®) and sertraline (Zoloft®); anti-psychotic agents, such as lithium (Lithobid®); and anticonvulsants, such as valproic acid (Depakene®), divalproex (Depakote®), and lamotrigine (Lamictal®); carbidopa-levodopa (Parcopa®); dopamine agonists, such as pramipexole (Mirapex®), ropinirole (Requip®), and apomorphine (Apokyn®); monoamine oxidase B inhibitors, such as selegiline (Eldepryl®, Zelapar®) and rasagiline (Azilect®); catechol O-methyltransferase (COMT) inhibitors, such as entacapone (Comtan®) and tolcapone (Tasmar®); anticholinergics, such as benztropine (Cogentin®) and trihexyphenidyl; and amantadine.

37. The method of any one of claims 30 to 36, wherein at least one LTK agonist is selected from an LTK agonist antibody, a FAM150A agent, and a FAM150B agent.

38. The method of any one of claims 30 to 36, wherein at least one LTK agonist is selected from a FAM150A agent and a FAM150B agent.

39. The method of any one of claims 30 to 38, wherein at least one LTK agonist is a FAM150A agent.

40. The method of claim 39, wherein the FAM150A agent comprises a sequence selected from SEQ ID NOs: 1 and 2.

41. The method of any one of claims 30 to 40, wherein at least one LTK agent is a FAM150B agent.

42. The method of claim 41, wherein the FAM150B agent comprises a sequence selected from SEQ ID NOs: 3 and 4.

43. The method of any one of claims 30 to 42, wherein at least one LTK agonist is a FAM150A fusion molecule.

44. The method of any one of claims 30 to 43, wherein at least one LTK agonist is a FAM150B fusion molecule.

45. The method of claim 43, wherein the FAM150A fusion molecule comprises FAM150A and at least one fusion partner.

46. The method of claim 44, wherein the FAM150B fusion molecule comprises FAM150B and at least one fusion partner.

47. The method of claim 45 or claim 46, wherein at least one fusion partner is selected from an Fc, albumin, and polyethylene glycol.

48. The method of any one of claims 45 to 47, wherein at least one fusion partner is an Fc.

49. The method of claim 48, wherein the Fc comprises a sequence selected from SEQ ID NOs: 17 to 19.

50. The method of any one of claims 45 to 47, wherein at least one fusion partner is polyethylene glycol.

51. Use of a molecule selected from a FAM150A antagonist, a FAM150B antagonist, and a FAM150A/B antagonist for treating cancer in a subject.

52. The use of claim 51, wherein the cancer is selected from lung cancer, leukemia, breast cancer, ovarian cancer, kidney cancer, colon cancer, and bladder cancer.

53. The use of claim 51, wherein the cancer is selected from non-small lung cancer, acute myeloid leukemia, and chronic lymphocytic leukemia.

54. Use of a molecule selected from a FAM150A antagonist, a FAM150B antagonist, and a FAM150A/B antagonist for treating an autoimmune condition in a subject.

55. The use of claim 54, wherein the autoimmune condition is selected from rheumatoid arthritis, systemic lupus erythematosus, ankylosing spondylitis, and multiple sclerosis.

56. The use of any one of claims 51 to 55, wherein the FAM150A antagonist selected from a FAM150A antibody, a leukocyte tyrosine kinase (LTK) antibody, an LTK extracellular domain (ECD), and an LTK ECD fusion molecule; the FAM150B antagonist is selected from a FAM150B antibody, a leukocyte tyrosine kinase (LTK) antibody, an LTK extracellular domain (ECD), and an LTK ECD fusion molecule; and the FAM150A/B antagonist is selected from a FAM150A/B antibody, a leukocyte tyrosine kinase (LTK) antibody, an LTK extracellular domain (ECD), an LTK ECD fusion molecule, and an ALK antibody.

57. The use of any one of claims 51 to 55, wherein the FAM150A antagonist is a FAM150A antibody, the FAM150B antagonist is a FAM150B antibody, and the FAM150A/B antagonist is a FAM150A/B antibody.

58. The use of any one of claims 51 to 57, wherein the antibody is selected from a chimeric antibody, a humanized antibody, and a human antibody.

59. The use of any one of claims 51 to 58 wherein the antibody is an antibody fragment.

60. The use of claim 59, wherein the antibody fragment is selected from an Fv, a single-chain Fv (scFv), a Fab, a Fab′, and a (Fab′)₂.

61. Use of at least one LTK agonist for treating a neurodegenerative disorder.

62. The use of claim 61, wherein the neurodegenerative disorder is selected from Huntington's disease, Parkinson's disease, and Alzheimer's disease.

63. The use of claim 61 or claim 62, wherein at least one LTK agonist is selected from an LTK agonist antibody, a FAM150A agent, and a FAM150B agent.

64. The use of any one of claims 61 to 63, wherein at least one LTK agonist is selected from a FAM150A agent and a FAM150B agent.

65. The use of claim 63 or claim 64, wherein the FAM150A agent comprises a sequence selected from SEQ ID NOs: 1 and 2; and the FAM150B agent comprises a sequence selected from SEQ NOs: 3 and 4.

66. The use of any one of claims 63 to 65, wherein the FAM150A agent is a FAM150A fusion molecule comprising FAM150A and at least one fusion partner; and wherein the FAM150B agent is a FAM150B fusion molecule comprising FAM150B and at least one fusion partner.

67. The use of claim 66, wherein at least one fusion partner is selected from an Fc, albumin, and polyethylene glycol.

68. The use of claim 67, wherein at least one fusion partner is an Fc.

69. A method of identifying a FAM150 antagonist, comprising:
a) contacting a candidate molecule with an LTK molecule and a FAM150 molecule, wherein the LTK molecule comprises LTK, an LTK ECD, or an LTK ECD fusion molecule, and the FAM150 molecule is selected from a FAM150A agent and a FAM150B agent; and
b) detecting binding of the LTK molecule to the FAM150 molecule;
wherein a reduction in the binding of the LTK molecule to the FAM150 molecule in the presence of the candidate molecule as compared to the binding of the LTK molecule to the FAM150 molecule in the absence of the candidate molecule indicates that the candidate molecule is a FAM150 antagonist.

70. The method of claim 69, wherein binding of the LTK molecule to the FAM150 molecule is reduced by at least 30%, at least 40%, at least 50%, at least 60%, at least 70%, or at least 80% in the presence of the candidate molecule.

71. The method of claim 69 or claim 70, wherein binding of the LTK molecule to the FAM150 molecule is detected by a method selected from surface plasmon resonance, ELISA, and flow cytometry.

72. A method of identifying a FAM150 antagonist, comprising:
a) contacting a candidate molecule with a cell expressing LTK and a FAM150 molecule, wherein the FAM150 molecule is selected from a FAM150A agent and a FAM150B agent; and
b) detecting phosphorylation of LTK;
wherein a reduction in phosphorylation of LTK in the presence of the candidate molecule as compared to the level of phosphorylation of LTK in the presence of the FAM150 molecule and the absence of the candidate molecule indicates that the candidate molecule is a FAM150 antagonist.

73. The method of claim 72, wherein phosphorylation of LTK is reduced by at least 30%, at least 40%, at least 50%, at least 60%, at least 70%, or at least 80% in the presence of the candidate molecule.

74. The method of claim 72 or claim 73, wherein phosphorylation of LTK is detected by a method selected from an immunoassay and a reporter assay.

75. The method of any one of claims 69 to 74, wherein the FAM150 antagonist is an antibody that binds to LTK.

76. The method any one of claims 69 to 74, wherein the FAM150 antagonist is an antibody that binds FAM150A and/or FAM150B.

77. The method of any one of claims 69 to 74, wherein the FAM150 antagonist is a small molecule.

78. A method of determining whether an LTK antibody is a FAM150 antagonist, comprising:
a) contacting the LTK antibody with an LTK molecule and a FAM150 molecule, wherein the LTK molecule comprises LTK, an LTK ECD, or an LTK ECD fusion molecule, and the FAM150 molecule is selected from a FAM150A agent and a FAM150B agent; and
b) detecting the binding of the LTK molecule to the FAM150 molecule;
wherein a reduction in the binding of the LTK molecule to the FAM150 molecule in the presence of the LTK antibody as compared to the binding of the LTK molecule to the FAM150 molecule in the absence of the LTK antibody indicates that the LTK antibody is a FAM150 antagonist.

79. The method of claim 78, wherein binding of the LTK molecule to the FAM150 molecule is reduced by at least 30%, at least 40%, at least 50%, at least 60%, at least 70%, or at least 80% in the presence of the LTK antibody.

80. The method of claim 78 or claim 79, wherein binding of the LTK molecule to the FAM150 molecule is detected by a method selected from surface plasmon resonance, ELISA, and flow cytometry.

81. A method of determining whether an LTK antibody is a FAM150 antagonist, comprising:
a) contacting the LTK antibody with a cell expressing LTK and a FAM150 molecule, wherein the FAM150 molecule is selected from a FAM150A agent and a FAM150B agent; and
b) detecting phosphorylation of LTK;
wherein a reduction in phosphorylation of LTK in the presence of the LTK antibody as compared to the level of phosphorylation of LTK in the presence of the FAM150 molecule and the absence of the LTK antibody indicates that the LTK antibody is a FAM150 antagonist.

82. The method of claim 81, wherein phosphorylation of LTK is reduced by at least 30%, at least 40%, at least 50%, at least 60%, at least 70%, or at least 80% in the presence of the LTK antibody.

83. The method of claim 81 or claim 82, wherein phosphorylation of LTK is detected by a method selected from an immunoassay and a reporter assay.


SEQUENCE LISTING (33 sequences)

SEQ ID NO: 1 - protein, 129 aa, Homo sapiens
MRPLKPGAPLPALFLLALALSPHGAHGRPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQT

SEQ ID NO: 2 - protein, 102 aa, Homo sapiens
RPRGRRGARVTDKEPKPLLFLPAAGAGRTPSGSRSAEIFPRDSNLKDKFIKHFTGPVTFSPECSKHFHRLYYNTRECSTPAYYKRCARLLTRLAVSPLCSQT

SEQ ID NO: 3 - protein, 152 aa, Homo sapiens
MRGPGHPLLLGLLLVLGAAGRGRGGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 4 - protein, 128 aa, Homo sapiens
GAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQ

SEQ ID NO: 5 - protein, 151 aa, Mus musculus
MRVSGRPMLLALLLLLTTVGDRGRAQSRGPADRQTLLRLLVELVQELKKFHIGDSKRLQLLGESDFALGRREATDYGADQEEQRVEIVPRDLRMKDKFLKHLTGPLYFSPKCSKHFHRLYHNTRDCTIPAYYKRCARLLTRLAVSPMCMER

SEQ ID NO: 6 - protein, 126 aa, Mus musculus
QSRGPADRQTLLRLLVELVQELKKFHIGDSKRLQLLGESDFALGRREATDYGADQEEQRVEIVPRDLRMKDKFLKHLTGPLYFSPKCSKHFHRLYHNTRDCTIPAYYKRCARLLTRLAVSPMCMER

SEQ ID NO: 7 - protein, 864 aa, Homo sapiens
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGPLVLMVAVVATSTLSLLMVCGVLILVKQKKWQGLQEMRLPSPELELSKLRTSAIRTAPNPYYCQVGLGPAQSWPLPPGVTEVSPANVTLLRALGHGAFGEVYEGLVIGLPGDSSPLQVAIKTLPELCSPQDELDFLMEALIISKFRHQNIVRCVGLSLRATPRLILLELMSGGDMKSFLRHSRPHLGQPSPLVMRDLLQLAQDIAQGCHYLEENHFIHRDIAARNCLLSCAGPSRVAKIGDFGMARDIYRASYYRRGDRALLPVKWMPPEAFLEGIFTSKTDSWSFGVLLWEIFSLGYMPYPGRTNQEVLDFVVGGGRMDPPRGCPGPVYRIMTQCWQHEPELRPSFASILERLQYCTQDPDVLNSLLPMELGPTPEEEGTSGLGNRSLECLRPPQPQELSPEKLKSWGGSPLGPWLSSGLKPLKSRGLQPQNLWNPTYRS

SEQ ID NO: 8 - protein, 848 aa, Homo sapiens
ILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGPLVLMVAVVATSTLSLLMVCGVLILVKQKKWQGLQEMRLPSPELELSKLRTSAIRTAPNPYYCQVGLGPAQSWPLPPGVTEVSPANVTLLRALGHGAFGEVYEGLVIGLPGDSSPLQVAIKTLPELCSPQDELDFLMEALIISKFRHQNIVRCVGLSLRATPRLILLELMSGGDMKSFLRHSRPHLGQPSPLVMRDLLQLAQDIAQGCHYLEENHFIHRDIAARNCLLSCAGPSRVAKIGDFGMARDIYRASYYRRGDRALLPVKWMPPEAFLEGIFTSKTDSWSFGVLLWEIFSLGYMPYPGRTNQEVLDFVVGGGRMDPPRGCPGPVYRIMTQCWQHEPELRPSFASILERLQYCTQDPDVLNSLLPMELGPTPEEEGTSGLGNRSLECLRPPQPQELSPEKLKSWGGSPLGPWLSSGLKPLKSRGLQPQNLWNPTYRS

SEQ ID NO: 9 - protein, 803 aa, Homo sapiens
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGDASETDNLWADGEDGVSFIHPSSELFLQPLAVTENHGEVEIRRHLNCSHCPLRDCQWQAELQLAECLCPEGMELAVDNVTCMDLHKPPGPLVLMVAVVATSTLSLLMVCGVLILVKQKKWQGLQEMRLPSPELELSKLRTSAIRTAPNPYYCQVGLGPAQSWPLPPGVTEVSPANVTLLRALGHGAFGEVYEGLVIGLPGDSSPLQVAIKTLPELCSPQDELDFLMEALIISKFRHQNIVRCVGLSLRATPRLILLELMSGGDMKSFLRHSRPHLGQPSPLVMRDLLQLAQDIAQGCHYLEENHFIHRDIAARNCLLSCAGPSRVAKIGDFGMARDIYRASYYRRGDRALLPVKWMPPEAFLEGIFTSKTDSWSFGVLLWEIFSLGYMPYPGRTNQEVLDFVVGGGRMDPPRGCPGPVYRIMTQCWQHEPELRPSFASILERLQYCTQDPDVLNSLLPMELGPTPEEEGTSGLGNRSLECLRPPQPQELSPEKLKSWGGSPLGPWLSSGLKPLKSRGLQPQNLWNPTYRS

SEQ ID NO: 10 - protein, 787 aa, Homo sapiens
ILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGDASETDNLWADGEDGVSFIHPSSELFLQPLAVTENHGEVEIRRHLNCSHCPLRDCQWQAELQLAECLCPEGMELAVDNVTCMDLHKPPGPLVLMVAVVATSTLSLLMVCGVLILVKQKKWQGLQEMRLPSPELELSKLRTSAIRTAPNPYYCQVGLGPAQSWPLPPGVTEVSPANVTLLRALGHGAFGEVYEGLVIGLPGDSSPLQVAIKTLPELCSPQDELDFLMEALIISKFRHQNIVRCVGLSLRATPRLILLELMSGGDMKSFLRHSRPHLGQPSPLVMRDLLQLAQDIAQGCHYLEENHFIHRDIAARNCLLSCAGPSRVAKIGDFGMARDIYRASYYRRGDRALLPVKWMPPEAFLEGIFTSKTDSWSFGVLLWEIFSLGYMPYPGRTNQEVLDFVVGGGRMDPPRGCPGPVYRIMTQCWQHEPELRPSFASILERLQYCTQDPDVLNSLLPMELGPTPEEEGTSGLGNRSLECLRPPQPQELSPEKLKSWGGSPLGPWLSSGLKPLKSRGLQPQNLWNPTYRS

SEQ ID NO: 11 - protein, 888 aa, Mus musculus
MGCSHRLLLWLGAAGTILCSNSEFQAPFLTPSLLPVLVLNSQEQKVTPTPSKLEPASLPNPLGTRGPWVFNTCGASGRSGPTQTQCDGAYTGSSVMVTVGAAGPLKGVQLWRAPDTGQYLISAYGAAGGKGAQNHLSRAHGIFLSAVFFLRRGEPVYILVGQQGQDACPGGSPESQLVCLGESGEHATTYGTERIPGWRRWAGGGGGGGGATSIFRLRAGEPEPLLVAAGGGGRSYRRRPDRGRTQAVPERLETRAAAPGSGGRGGAAGGGSGWTSRAHSPQAGRSPREGAEGGEGCAEAWAALRWAAAGGFGGGGGACAAGGGGGGYRGGDTSESDLLWADGEDGTSFVHPSGELYLQPLAVTEGHGEVEIRKHPNCSHCPFKDCQWQAELWTAECTCPEGTELAVDNVTCMDLPTTASPLILMGAVVAALALSLLMMCAVLILVNQKCQGLWGTRLPGPELELSKLRSSAIRTAPNPYYCQVGLSPAQPWPLPPGLTEVSPANVTLLRALGHGAFGEVYEGLVTGLPGDSSPLPVAIKTLPELCSHQDELDFLMEALIISKFSHQNIVRCVGLSFRSAPRLILLELMSGGDMKSFLRHSRPHPGQLAPLTMQDLLQLAQDIAQGCHYLEENHFIHRDIAARNCLLSCSGASRVAKIGDFGMARDIYQASYYRKGGRTLLPVKWMPPEALLEGLFTSKTDSWSFGVLLWEIFSLGYMPYPGHTNQEVLDFIATGNRMDPPRNCPGPVYRIMTQCWQHQPELRPDFGSILERIQYCTQDPDVLNSPLPMEPGPILEEEEASRLGNRSLEGLRSPKPLELSSQNLKSWGGGLLGSWLPSGLKTLKPRCLQPQNIWNPTYGSWTPRGPQGEDTGIEQCNGSSSSSIPGIQ

SEQ ID NO: 12 - protein, 872 aa, Mus musculus
ILCSNSEFQAPFLTPSLLPVLVLNSQEQKVTPTPSKLEPASLPNPLGTRGPWVFNTCGASGRSGPTQTQCDGAYTGSSVMVTVGAAGPLKGVQLWRAPDTGQYLISAYGAAGGKGAQNHLSRAHGIFLSAVFFLRRGEPVYILVGQQGQDACPGGSPESQLVCLGESGEHATTYGTERIPGWRRWAGGGGGGGGATSIFRLRAGEPEPLLVAAGGGGRSYRRRPDRGRTQAVPERLETRAAAPGSGGRGGAAGGGSGWTSRAHSPQAGRSPREGAEGGEGCAEAWAALRWAAAGGFGGGGGACAAGGGGGGYRGGDTSESDLLWADGEDGTSFVHPSGELYLQPLAVTEGHGEVEIRKHPNCSHCPFKDCQWQAELWTAECTCPEGTELAVDNVTCMDLPTTASPLILMGAVVAALALSLLMMCAVLILVNQKCQGLWGTRLPGPELELSKLRSSAIRTAPNPYYCQVGLSPAQPWPLPPGLTEVSPANVTLLRALGHGAFGEVYEGLVTGLPGDSSPLPVAIKTLPELCSHQDELDFLMEALIISKFSHQNIVRCVGLSFRSAPRLILLELMSGGDMKSFLRHSRPHPGQLAPLTMQDLLQLAQDIAQGCHYLEENHFIHRDIAARNCLLSCSGASRVAKIGDFGMARDIYQASYYRKGGRTLLPVKWMPPEALLEGLFTSKTDSWSFGVLLWEIFSLGYMPYPGHTNQEVLDFIATGNRMDPPRNCPGPVYRIMTQCWQHQPELRPDFGSILERIQYCTQDPDVLNSPLPMEPGPILEEEEASRLGNRSLEGLRSPKPLELSSQNLKSWGGGLLGSWLPSGLKTLKPRCLQPQNIWNPTYGSWTPRGPQGEDTGIEQCNGSSSSSIPGIQ

SEQ ID NO: 13 - protein, 424 aa, Homo sapiens
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGP

SEQ ID NO: 14 - protein, 408 aa, Homo sapiens
ILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGP

SEQ ID NO: 15 - protein, 421 aa, Mus musculus
MGCSHRLLLWLGAAGTILCSNSEFQAPFLTPSLLPVLVLNSQEQKVTPTPSKLEPASLPNPLGTRGPWVFNTCGASGRSGPTQTQCDGAYTGSSVMVTVGAAGPLKGVQLWRAPDTGQYLISAYGAAGGKGAQNHLSRAHGIFLSAVFFLRRGEPVYILVGQQGQDACPGGSPESQLVCLGESGEHATTYGTERIPGWRRWAGGGGGGGGATSIFRLRAGEPEPLLVAAGGGGRSYRRRPDRGRTQAVPERLETRAAAPGSGGRGGAAGGGSGWTSRAHSPQAGRSPREGAEGGEGCAEAWAALRWAAAGGFGGGGGACAAGGGGGGYRGGDTSESDLLWADGEDGTSFVHPSGELYLQPLAVTEGHGEVEIRKHPNCSHCPFKDCQWQAELWTAECTCPEGTELAVDNVTCMDLPTTASP

SEQ ID NO: 16 - protein, 405 aa, Mus musculus
ILCSNSEFQAPFLTPSLLPVLVLNSQEQKVTPTPSKLEPASLPNPLGTRGPWVFNTCGASGRSGPTQTQCDGAYTGSSVMVTVGAAGPLKGVQLWRAPDTGQYLISAYGAAGGKGAQNHLSRAHGIFLSAVFFLRRGEPVYILVGQQGQDACPGGSPESQLVCLGESGEHATTYGTERIPGWRRWAGGGGGGGGATSIFRLRAGEPEPLLVAAGGGGRSYRRRPDRGRTQAVPERLETRAAAPGSGGRGGAAGGGSGWTSRAHSPQAGRSPREGAEGGEGCAEAWAALRWAAAGGFGGGGGACAAGGGGGGYRGGDTSESDLLWADGEDGTSFVHPSGELYLQPLAVTEGHGEVEIRKHPNCSHCPFKDCQWQAELWTAECTCPEGTELAVDNVTCMDLPTTASP

SEQ ID NO: 17 - protein, 232 aa, Artificial sequence
  Synthetic
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 228 aa, Artificial sequence
  Synthetic
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 229 aa, Artificial sequence
  Synthetic
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 21 - protein, 1602 aa, Homo sapiens
VGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 22 - protein, 659 aa, Artificial sequence
  Synthetic
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGPLGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 643 aa, Artificial sequence
  Synthetic
ILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGGGGWTSRAPSPQAGRSLQEGAEGGQGCSEAWATLGWAAAGGFGGGGGACTAGGGGGGYRGGDASETDNLWADGEDGVSFIHPSSELFLQPLAVTENHGEVEIRRHLNCSHCPLRDCQWQAELQLAECLCPEGMELAVDNVTCMDLHKPPGPLGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 1272 aa, Artificial sequence
  Synthetic
MGAIGLLWLLPLLLSTAAVGSGMGTGQRAGSPAAGSPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 1254 aa, Artificial sequence
  Synthetic
VGSGMGTGQRAGSPAAGSPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 655 aa, Artificial sequence
  Synthetic
MGCSHRLLLWLGAAGTILCSNSEFQTPFLTPSLLPVLVLNSQEQKVTPTPSKLEPASLPNPLGTRGPWVFNTCGASGRSGPTQTQCDGAYTGSSVMVTVGAAGPLKGVQLWRVPDTGQYLISAYGAAGGKGAQNHLSRAHGIFLSAVFFLRRGEPVYILVGQQGQDACPGGSPESQLVCLGESGEHATTYGTERIPGWRRWAGGGGGGGGATSIFRLRAGEPEPLLVAAGGGGRSYRRRPDRGRTQAVPERLETRAAAPGSGGRGGAAGGGSGWTSRAHSPQAGRSPREGAEGGEGCAEAWAALRWAAAGGFGGGGGACAAGGGGGGYRGGDTSESDLLWADGEDGTSFVHPSGELYLQPLAVTEGHGEVEIRKHPNCSHCPFKDCQWQAELWTAECTCPEGTELAVDNVTCMDLPTTASPGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 639 aa, Artificial sequence
  Synthetic
ILCSNSEFQTPFLTPSLLPVLVLNSQEQKVTPTPSKLEPASLPNPLGTRGPWVFNTCGASGRSGPTQTQCDGAYTGSSVMVTVGAAGPLKGVQLWRVPDTGQYLISAYGAAGGKGAQNHLSRAHGIFLSAVFFLRRGEPVYILVGQQGQDACPGGSPESQLVCLGESGEHATTYGTERIPGWRRWAGGGGGGGGATSIFRLRAGEPEPLLVAAGGGGRSYRRRPDRGRTQAVPERLETRAAAPGSGGRGGAAGGGSGWTSRAHSPQAGRSPREGAEGGEGCAEAWAALRWAAAGGFGGGGGACAAGGGGGGYRGGDTSESDLLWADGEDGTSFVHPSGELYLQPLAVTEGHGEVEIRKHPNCSHCPFKDCQWQAELWTAECTCPEGTELAVDNVTCMDLPTTASPGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 597 aa, Artificial sequence
  Synthetic
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGDASETDNLWADGEDGVSFIHPSSELFLQPLAVTENHGEVEIRRHLNCSHCPLRDCQWQAELQLAECLCPEGMELAVDNVTCMDLHKPPGPGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 581 aa, Artificial sequence
  Synthetic
ILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGDASETDNLWADGEDGVSFIHPSSELFLQPLAVTENHGEVEIRRHLNCSHCPLRDCQWQAELQLAECLCPEGMELAVDNVTCMDLHKPPGPGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 363 aa, Homo sapiens
MGCWGQLLVWFGAAGAILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGDASETDNLWADGEDGVSFIHPSSELFLQPLAVTENHGEVEIRRHLNCSHCPLRDCQWQAELQLAECLCPEGMELAVDNVTCMDLHKPPGP

SEQ ID NO: 31 - protein, 347 aa, Homo sapiens
ILCSSPGSQETFLRSSPLPLASPSPRDPKVSAPPSILEPASPLNSPGTEGSWLFSTCGASGRHGPTQTQCDGAYAGTSVVVTVGAAGQLRGVQLWRVPGPGQYLISAYGAAGGKGAKNHLSRAHGVFVSAIFSLGLGESLYILVGQQGEDACPGGSPESQLVCLGESRAVEEHAAMDGSEGVPGSRRWAGGGGGGGGATYVFRVRAGELEPLLVAAGGGGRAYLRPRDRGRTQASPEKLENRSEAPGSGGRGGAAGGDASETDNLWADGEDGVSFIHPSSELFLQPLAVTENHGEVEIRRHLNCSHCPLRDCQWQAELQLAECLCPEGMELAVDNVTCMDLHKPPGP

SEQ ID NO: 32 - protein, 127 aa, Mus musculus
MWLTKPSTPVSALLLLALALSPPGTQGRPQRSLAARVAELRPELFLPVTGTRLPPRASRSTEIFPRDLTLKDKFIKHFTGPVTFSAECSKHFHRLYHNTRDCSTPAYYKRCARLLTRLAVSPLCSQT

SEQ ID NO: 33 - protein, 100 aa, Mus musculus
RPQRSLAARVAELRPELFLPVTGTRLPPRASRSTEIFPRDLTLKDKFIKHFTGPVTFSAECSKHFHRLYHNTRDCSTPAYYKRCARLLTRLAVSPLCSQT